(19) United States
(12) Patent Application Publication
(10) Pub. No.: US 2025/0333774 A1
(43) Pub. Date: Oct. 30, 2025

(54) Saponin Production

(71) Applicant: GlaxoSmithKline Biologicals SA, Rixensart (BE)

(72) Inventor: Gregory MATHY, Rixensart (BE)

(73) Assignee: GlaxoSmithKline Biologicals SA, Rixensart (BE)

(21) Appl. No.: 18/702,436

(22) PCT Filed: Oct. 17, 2022
(86) PCT No.: PCT/EP2022/078884

§ 371 Date: Apr. 18, 2024

(30) Foreign Application Priority Data
Oct. 19, 2021 (EP) 21203583.6
Jun. 7, 2022 (GB) 2208339.8

(51) Int. Cl. C12P 19/56; C12N 5/04

(52) U.S. Cl. CPC C12P 19/56; C12N 5/04

(57) ABSTRACT

The invention relates to a method for producing saponins containing a quillaic acid triterpenoid aglycone, said method comprising at least the following steps:
i) culturing plant cells capable of naturally synthesizing saponins containing a quillaic acid triterpenoid aglycone in a cell culture medium comprising a source of nitrogen,
ii) depleting the culture medium from any nitrogen source,
iii) eliciting the production of saponins with at least one elicitor, and
iv) recovering the saponins produced.

### CROSS-REFERENCE TO RELATED APPLICATIONS

This application is a National Stage Application under 35 U.S.C. § 371 of International Application No. PCT/EP2022/078884, filed Oct. 17, 2022, which claims the benefit under 35 U.S.C. § 119(b) of each of European Application No. 21203583.6.1, filed Oct. 19, 2021, and GB Application No. 2208339.8, filed Jun. 7, 2022; each of the above-identified applications are hereby incorporated herein by reference in their entireties.

### FIELD OF THE INVENTION

The present invention generally relates to saponin production in plant cell culture, in particular, saponins containing a quillaic acid triterpenoid aglycone. In particular, the invention relates to plant cells capable of producing such saponins, methods for producing such saponins, and associated aspects.

### BACKGROUND

Saponins are triterpenoid glycosides. They have a broad range of uses from fire extinguisher foams to food additives and immunostimulants (Reichert et al., 2019). Saponins have been of interest as immunostimulants for many decades. Traditionally, saponins are purified from plants, such as for example Quillaja saponaria Molina trees. For example, Quil A is a saponin preparation isolated from the South American tree Quillaja Saponaria Molina and was first described as having adjuvant activity by Dalsgaard et al. in 1974. Purified fractions of Quil A have been isolated by HPLC which retain adjuvant activity without the toxicity associated with Quil A (see, for example, EP03622789). Various fractions have been found to have adjuvant activity, such as the fractions QS-7, QS-17, QS-18 and QS-21, although their toxicity varies considerably. While QS-18 is the most abundant saponin fraction (Kensil et al. 1991), QS-7 and QS-21 were found less toxic in mice. QS-21, being more abundant than QS-7, the QS-21 fraction has been the most widely studied saponin adjuvant (Ragupathi et al. 2011).

An example of an adjuvant formulation including QS-21 is the Adjuvant System 01 (ASO1), which is a liposome-based adjuvant which contains two immunostimulants, 3-O-desacyl-4′-monophosphoryl lipid A (3D-MPL) and QS-21 (Garcon, 2011; Didierlaurent, 2017). 3D-MPL is a non-toxic derivative of the lipopolysaccharide from Salmonella minnesota. ASO1 is included in vaccines for malaria (RTS,S—Mosquirix™) and Herpes zoster (HZ/su—Shingrix™), and in multiple candidate vaccines. ASO1 injection results in rapid and transient activation of innate immunity in animal models. Neutrophils and monocytes are rapidly recruited to the draining lymph node (dLN) upon immunization. Moreover, ASO1 induces recruitment and activation of MHCII^{high} dendritic cells (DC), which are necessary for T cell activation (Didierlaurent et al., 2014). Some data are also available on the mechanism of action of the components of ASO1. 3D-MPL signals via TLR4, stimulating NF-κB transcriptional activity and cytokine production and directly activates antigen-presenting cells (APCs) both in humans and in mice (De Becker et al. 2000; Ismaili et al. 2002; Martin, 2003; Mata-Haro, 2007). QS-21 promotes high antigen-specific antibody responses and CD8′ T-cell responses in mice (Kensil, 1998; Newman, 1992; Soltysik, 1995) and antigen-specific antibody responses in humans (Livingston, 1994). Because of its physical properties, it is thought that QS-21 might act as a danger signal in vivo (Lambrecht, 2009; Li, 2008). Although QS-21 has been shown to activate ASC-NLRP3 inflammasome and subsequent IL-1(3/IL-18 release (Marty-Roix, 2016), the exact molecular pathways involved in the adjuvant effect of saponins have yet to be clearly defined. Another example of an adjuvant formulation including QS-7 is Matrix M (as part of the saponin fraction named “Fraction A”—see e.g. WO 2011/161151) which is an ISCOM-based formulation included in the vaccine against COVID-19 (Nuvaxovid™)

Extracts of Quillaja saponaria are commercially available, including fractions thereof with differing degrees of purity such as Quil A, Fraction A, Fraction B, Fraction C, QS-7, QS-17, QS-18 and QS-21. Such extracts typically originate from the harvesting of bark from Quillaja saponaria trees.

Because the current source for saponins is dependent on natural resources, and such natural resources may be limiting, there is a need to develop alternative and sustainable methods for producing saponins which rely less upon natural resources, such as producing saponins in plant cell culture.

WO 94/10291 discloses cultured cells of Quillaja saponaria and methods for preparing saponins for use as active substances useful as adjuvants. However, the inventors observed that when using the methods disclosed in WO 94/10291, not only saponins were not always produced, but also, even when produced, the level achieved was low and not reproducible showing some variability. Therefore, there remains a need for developing methods of producing saponins in plant cell culture capable of producing a high level of saponins in a robust, reliable and consistent manner.

### SUMMARY OF THE INVENTION

In one aspect of the invention, there is provided a method for converting non-producing plant cells capable of naturally synthesizing saponins containing a quillaic acid triterpenoid aglycone into plant cells producing saponins (and cells obtainable by the method), said method comprising at least the following steps:
i) culturing plant cells capable of naturally synthesizing saponin containing a quillaic acid triterpenoid aglycone in a culture medium comprising a source of nitrogen,
ii) depleting the culture medium from any nitrogen source, and
iii) eliciting the production of saponins with at least one elicitor.

In another aspect of the invention, there is provided a method for producing saponins containing a quillaic acid triterpenoid aglycone, said method comprising at least the following steps:
i) culturing plant cells capable of naturally synthesizing saponin containing a quillaic acid triterpenoid aglycone in a culture medium comprising a source of nitrogen,
ii) depleting the culture medium from any nitrogen source,
iii) eliciting the production of saponins with at least one elicitor, and
iv) recovering the saponins produced.

In a further aspect of the invention, there is provided a suspension of plant cells naturally synthesizing saponins containing a quillaic acid triterpenoid aglycone capable of producing such saponins with a volume productivity of at least 5 mg of saponins/L of cell culture.

In a further aspect of the invention, there is provided a suspension cell line of plant cells naturally synthesizing saponins containing a quillaic acid triterpenoid aglycone capable of producing such saponins with a volume productivity of at least 5 mg of saponins/L of cell culture.

In a further aspect of the invention, there is provided a method for preparing an adjuvant comprising saponins, said method comprising the steps of (a) preparing saponins according to the method of the invention and (b) formulating the saponins as an adjuvant.

### BRIEF DESCRIPTION OF THE DRAWINGS

FIG. 1 shows the evolution of growth (PCV %) of a suspension cell line of plant cells cultured in different culture media, each comprising different sources of nitrogen (as indicated), followed by elicitation (as indicated).

FIG. 2 shows the QS-18 volumetric productivity calculated from plant cell extracts obtained from one suspension cell line of plant cells after 3 days, 5 days or 7 days of nitrogen depletion (as indicated), followed by a 2-day, 4-day or 7-day elicitation with different concentrations of MeJa (as indicated).

FIG. 3 shows the QS-18 volumetric productivity calculated from plant cell extracts obtained from one suspension cell line of plant cells after 5 days of nitrogen depletion, followed by a 2-day, 4-day or 7-day elicitation with different concentrations of MeJa (as indicated).

FIG. 4 shows a comparison of the QS-18 volumetric productivity calculated from plant cell extracts obtained from 3 different suspension cell lines of plant cells after 5 days of nitrogen depletion, followed by a 4-day elicitation with different concentrations of MeJa (as indicated).

FIG. 5A shows an HPLC-ELSD chromatogram of the QS-18 standard. A single peak is obtained at the indicated retention time.

FIG. 5B shows a high-resolution LCMS chromatogram using QToF mass spectrometer. Saponin species (including QS-18 saponin species) included and identified in the QS-18 standard are named next to the peak corresponding to the indicated retention time (RT).

FIG. 6 shows an HPLC-ELSD chromatogram of the QS-21 standard. A single peak is obtained at the indicated retention time.

FIG. 7 shows an HPLC-ELSD chromatogram representative of a plant cell extract obtained from one suspension cell line of plant cells (CMC40B6) cultured in conditions allowing to produce saponins (e.g. here 5 days of nitrogen depletion, followed by a 4-day elicitation with 2.8 μM MeJa/PCV %). The peak corresponding to the QS-17 family of saponins, the peak corresponding to the QS-18 family of saponins and the peak corresponding to QS-21 family of saponins are indicated, the retention time of each peak being similar to the retention time at which the respective standard peaks.

FIG. 8 shows an HPLC-ELSD chromatogram representative of a plant cell extract obtained from one suspension cell line of plant cells (CMC40B6) cultured in conditions where no saponin is produced (e.g. here 5 days of nitrogen depletion with no subsequent elicitation).

FIG. 9 shows a comparison of the QS-21 volumetric productivity calculated from plant cell extracts obtained from 3 different suspension cell lines of plant cells after 5 days of nitrogen depletion, followed by a 7-day elicitation (CMC16B and CMC35A8) or 8-day elicitation (CMC40B6) with different concentrations of MeJa (as indicated).

FIG. 10 shows a comparison of the QS-21 volumetric productivity calculated from plant cell extracts obtained from the CMC16B suspension cell line. Cells were cultured under the following different conditions: (i) no depletion/no elicitation, (ii) no depletion/elicitation, (iii) depletion/no elicitation, and (iv) depletion/elicitation (as indicated).

FIG. 11 shows the QS-21 volumetric productivity calculated from a plant cell extract obtained from the CMC5B-1 suspension cell line after 5 days of nitrogen depletion, followed by a 5-day elicitation with 3.3 μM MeJa/PCV %.

FIG. 12A shows a high-resolution LCMS chromatogram of a QS-21 standard using QToF mass spectrometer. QS-21 saponin species (including QS-21 1988) included and identified in the standard are named next to the peak corresponding to the indicated retention time (RT).

FIG. 12B shows an LCMS-MS chromatogram for the content of QS-21 1988 (as A V1 and A V2 isomers) in the standard.

FIGS. 12A-13C show the QS-18 volumetric productivity calculated from plant cell extracts obtained from one suspension cell line of plant cells (CMC16B) after 5 days of nitrogen depletion, followed by a 4-day, 7-day, 10-day, or 14-dab elicitation with 8 μM MeJa/PCV %. A range of different reduced concentrations of nitrogen source has been tested during the nitrogen depletion phase (as indicated).

FIGS. 14A and 14B show UPLC/MS chromatograms using QToF mass spectrometer for the detection of QS-7 1862. Comparison of plant cell extracts obtained from the CMC16B suspension cell line cultured under two different conditions: no depletion/no elicitation (Panel A), and depletion/elicitation (Panel B).

FIGS. 15A-15D show the QS-21 volumetric productivity calculated from plant cell extracts obtained from the CMC40B6 suspension cell line (FIG. 15A). Cells were let naturally consume the source of nitrogen in the culture medium down to a residual level, and then further maintained in the consumed medium for 5 more days before being elicited (D14) with 2 μM MeJa/PCV % for 7 days. The level of ammonium and the level of nitrates, as monitored in the culture medium during this experiment, are shown in FIG. 14B and FIG. 14C, respectively. FIG. 15D shows the QS-21 volumetric productivity calculated from a plant cell extract obtained from the same culture of CMC40B6, but subject to a nitrogen depletion by replacing the culture medium with a culture medium including no source of nitrogen and maintaining the cells in the medium for 5 days, before eliciting the cells in the same conditions (2 μM MeJa/PCV % for 7 days).

### DETAILED DESCRIPTION OF THE INVENTION

The present inventors have developed culture conditions which allow production of saponins containing a quillaic acid triterpenoid aglycone with improved yield and/or consistency, such as at least about 5 to 10 times higher than when using conventional methods in the art (e.g. a volumetric productivity of saponins of at least 10 mg/L of culture medium, and up to 50 mg/L, is achieved by the method of the invention). Surprisingly, using these culture conditions, even plant cells which did not produce saponins using the methods of the prior art could achieve saponin production. While it has been shown that in some plant cells naturally synthesizing some triterpenoid saponins, the production of such saponins may be triggered by elicitation (Yendo et al., 2010), the inventors observed that the physiological state of the plant cells was also a key factor in controlling saponin production. As a result, the inventors developed a method suitable for the production of saponins containing a quillaic acid triterpenoid aglycone involving three distinct phases: (i) an expansion phase aimed at providing a desired level of cell biomass, (ii) a nitrogen depletion phase aimed at increasing the susceptibility of the cells to subsequent elicitation, and (iii) an elicitation phase aimed at triggering the saponin production.

In the context of the present invention, the term “saponin” is to be understood as referring to triterpenoid glycosides, the triterpenoid core (or aglycone) of which being quillaic acid. Such saponins may alternatively be referred to as “saponins containing a quillaic acid triterpenoid aglycone”.

In the context of the invention, the term “plant cell culture” or “plant cells” is to be understood as the in vitro culture of any plant tissues or any plant cell types. The plant cells used in the method of the invention originate from any plant naturally synthesizing saponins containing a quillaic acid triterpenoid aglycone. The plant may belong to the genus Quillaja, such as for instance, the species Quillaja saponaria, or Quillaja brasiliensis. Alternatively, the plant may belong to the genus Saponaria, such as for example, the species Saponaria vaccaria, or Saponaria officinalis. In one embodiment, the method of the invention uses plant cells originating from the genus Quillaja. In a further embodiment, the plant cells originate from the species Quillaja Saponaria. In a further alternative embodiment, the plant cells originate from the species Quillaja brasiliensis.

The method of the invention is applicable to any type of cultivation vessels adapted to the type of cells cultured, of any size, such as for example, petri dishes, shake flasks, or bioreactors. Bioreactors may include disposable bioreactors, typically comprising plastic bags, or non-disposable bioreactors, such as stainless steel bioreactors. In one embodiment, culture disposable bioreactors are used. In an alternative embodiment, non-disposable bioreactors are used. In a further alternative embodiment, shake flasks are used.

The conventional culture media known for plant cell culture, such as classical Murashige and Skoog (MS) media, can be used in the method of the invention. These media typically contain at least one or more macronutrients, e.g. selected from NH₄NO₃, KNO₃, CaCl₂, MgSO₄, KH₂PO₄, NH₄Cl, or KCl; at least one or more micronutrients, e.g. selected from KI, H₃BO₃, MnSO₄, ZnSO₄, Na₂MoO₄, CuSO₄, CoCl₂, DeSO₄, or Na₂EDTA; at least one or more vitamins, e.g. selected from myo-inositol, nicotinic acid, pyrodixine-HCl, or thiamine-HCl, for example at a total concentration between 0.01 and 3 g/L, such as between 50 and 150 mg/L; optionally one or more amino acids, such as glycine; at least one or more carbon source, e.g. selected from sucrose, glucose or fructose; and at least one or more plant hormones, e.g. selected from one or more cytokinins, such as 6- Benzylaminopurine (BA), or one or more auxins, such as 2,4-dichlorophenoxyacetic acid (2,4-D) and/or 1-Napthaleneacetic acid (NAA).

The replenishment of fresh culture medium, or selected nutrients which may have been consumed, to cells undergoing growth or active biosynthesis, such as during the production of saponins, may also enhance production and/or be necessary, e.g. replenishment of the carbon source and/or phosphate source may be useful in the method of the invention.

It is contemplated that the amount of medium exchanged or replenished, the frequency of exchange, and the composition of medium being replenished can be varied, in accordance with various embodiments of the invention. This may vary depending on the phase of the method of the invention. Replenishment may take place in a continuous, semi-continuous, or fed-batch mode. In a fed-batch process, particular medium components, such as selected nutrients are supplied either periodically or continuously. Suitably, a substantial portion, but not all, of the contents of a batch culture is replaced by fresh medium for continued cell growth and saponin production. Alternatively, the process is “continuous”, that is, fresh medium is continuously supplied, and effluent medium is continuously or repetitively removed. In one embodiment, replenishment of fresh culture medium or selected nutrients is supplied in the method of the invention by fed-batch, e.g. during step i), during step ii) and/or during step iii) of the method of the invention.

The method of the invention is applicable to any type of plant materials cultured in vitro, such as cells, tissues or organs of a given plant body, e.g. primordia, leaves, stems, hairy roots, internodes, cambium, whether cultured in suspension in a liquid medium or on a solid medium, e.g. calli. In one embodiment, the plant cells used in the method of the invention originate from the cambium, e.g. are cambial meristematic cells (CMC). In an alternative embodiment, the plant cells originate from hairy roots.

The plant cells used in the method of the invention may be a callus, e.g. deriving from the cambium of the plant. In the context of the invention, “callus” is to be understood as a cluster of dedifferentiated cells cultured on solidified medium. Callus generation may be achieved from any plant tissue explant by any method known to the skilled person, e.g. the methods described in WO 94/10291, in US 2019/0134128 or in WO 15/082978. Typically, tissue explants from a plant of a small size may be surface sterilized, e.g. by washing thoroughly with clean water, using a disinfectant such as hypochlorite, using wett agents, such as Tween or Triton, using antibiotics and/or using anti-fungal agents. Surface sterilized explants are then, typically, laid on the surface of solidified medium, such as agar, and incubated in a sterile environment, until a mass of undifferentiated cells grows (typically between 2 to 12 weeks, e.g. 8 weeks) in proximity to the plant source material. Calli may be gradually purified and further propagated by means of repeating the similar solid medium-culturing, that is, by inoculating fresh solid medium by turns with small pieces of callus formed in the previous solid medium-culturing, e.g. every 4 weeks. In one embodiment, calli are cultured in the presence of hormones 1-Naphthaleneacetic acid (NAA) and 6- Benzylaminopurine (BA), e.g. at 0.5 mg/L.

Calli thus formed and refined on the solid medium by subculture may be inoculated into a liquid medium and cultured so as to obtain a suspension cell culture. The terms “suspension plant culture” and “suspension of plant cells” are interchangeable and refer to an in vitro culture of plant cells dispersed in a liquid medium. The method of the invention is particularly suitable for suspension plant cultures or suspension of plant cells. Accordingly, in one embodiment, the plant cells for use in the method of the invention are grown in suspension in a liquid medium. In the context of the invention, the term “cell line” refers to plant cells originating from a given callus and which have been adapted to grow in suspension in a liquid culture medium. Different suspension cell lines may be established from a given callus. To obtain cells into a suspension culture, such as the suspension cell lines of plant cells of the invention, cells are for example removed from a callus and transferred to sterile culture vessels containing nutrient culture medium. It is appreciated that optimized media for suspension cell lines may differ from the optimum for callus. It is within the ambit of the skilled person to determine suitable and optimal culture media.

The transition from a callus to suspension cell lines is also known to the skilled person (described e.g. in WO 94/10291 or US 2019/0134128). The inventors observed that the use of conditioned medium (i.e. a culture medium in which some cells have been previously grown and therefore containing components secreted by the previous cells) and/or phytosulfokine alpha (PSK) may help transitioning from calli to suspension cultures. Accordingly, in one embodiment, conditioned medium and/or PSK are included in the culture medium when transitioning from calli to suspension cell lines and/or sub-culturing suspension cell lines. Once initiated and adapted to growth in suspension, suspension cell lines may be sub-cultured or propagated, for example by dilution, e.g. every 4 weeks, so as to maintain them in a state of growth and proliferation, prior to step i) of the method of the invention.

Step i)—Culturing Plant Cells in a Cell Culture Medium Comprising a Source of Nitrogen

In one embodiment, the plant cells in step i) of the method of the invention are a callus. In a preferred embodiment, the plant cells in step i) of the method of the invention are grown in suspension or are suspension cell lines.

The plant cells in step i) are cultured and maintained in conditions allowing proliferation and growth until a desired cell biomass is achieved. This is the expansion phase. For example, when the plant cells are grown in suspension or are suspension cell lines, the cell biomass may be assessed by measuring the PCV. The term “PCV” stands for Packed Cell Volume and refers to the volume occupied by cells in culture medium. It may be calculated as follows: PCV (%)=(volume of cell pellet/volume of sample)×100. A suitable PCV range achieved at the end of step i) may be between 10% and 70%, more suitably between 20% and 60%, and even more suitably, between 30% and 50%, e.g. about 40%. In other words, the plant cells in step i) may be cultured until reaching a PCV suitably ranging between 10% and 70%, more suitably between 20% and 60%, and even more suitably, between 30% and 50%, e.g. 40%. In one embodiment, the PCV at the end of step i) is about 15%, about 20%, about 30%, about 40%, about 50%, or about 60%. Alternatively, the above PCV ranges or values may be achieved by appropriate dilution of the plant cells which have been cultured and maintained in step i) before starting step ii). For example, plant cells cultured in a culture medium comprising a source of nitrogen (as described below) in step i) are centrifuged, and the desired cell biomass is resuspended directly into a culture medium containing no source of nitrogen, or a reduced source of nitrogen, so as to obtain a desired PCV range or values when starting step ii).

The duration of step i) may vary from one cell line to another, depending on their growth rate and depending on the desired PCV range or value to be reached. Suitably, step i) may last for 4 to 8 days, more suitably, for 5 to 7 days, even more suitably for 4 to 5 days, or longer.

Culture media suitable for use in step i) may be variations of the classic MS medium, such as an increased concentration of phosphate source (e.g. KH₂PO₄) and/or a modified sugar balance (e.g. glucose and fructose versus sucrose).

In some embodiments, the culture medium in step i) comprises at least KH₂PO₄ between 2 mM and 4 mM, or between 0.6 mM and 5 mM, or between 1.5 mM and 5 mM. In a further embodiment, the culture medium in step i) comprises at least KH₂PO₄ at about 2.5 mM or about 1.25 mM.

In the context of the invention, the term “source of nitrogen” encompasses nitrates (i.e. a source of NO³⁻ ions, such as e.g. KNO₃ or NH₄NO₃) and/or ammonium (i.e. a source of NH⁴⁺ ions, such as e.g. NH₄Cl or NH₄NO₃).

Both nitrate and ammonium are known to support growth of plant cells.

Advantageously, the source of nitrogen in the culture medium in step i) suitably includes at least nitrates, such as KNO₃. In some embodiments, the source of nitrogen includes at least KNO₃. In further embodiments, the source of nitrogen includes at least KNO₃ and NH₄NO₃. In further embodiments, the source of nitrogen may optionally include NH₄Cl. In further embodiments, the source of nitrogen does not include NH₄Cl as the sole source of nitrogen.

The total concentration of the nitrogen source in the culture medium in step i) may range from 10 mM to 50 mM, suitably from 15 mM to 40 mM, more suitably from 20 mM to 30 mM, e.g. may be about 25 mM, about 30 mM or about 40 mM. The concentration of KNO₃ (when present) may range from 5 mM to 30 mM, suitably from 10 mM to 20 mM, more suitably, may be about 15 mM or about 20 mM. The concentration of NH₄NO₃ (when present) may range from 5 mM to 30 mM, suitably from 10 mM to 20 mM, more suitably may be about 10 mM or about 20. The concentration of NH₄Cl (when present) may range from 5 mM to 30 mM, suitably from 5 mM to 20 mM, more suitably from 10 mM to 20 mM, and more suitably, may be about 10 mM or about 15 mM.

The source of carbon in the culture medium in step i) may be one or more of sucrose, glucose and fructose, in particular, may suitably be a combination of sucrose, glucose and fructose. The total concentration of the carbon source may range from 40 mM to 100 mM, suitably from 50 mM to 90 mM, more suitably from 60 mM to 80 mM, e.g. may be about 60 mM or about 70 mM. The concentration of sucrose (when present) may range from 5 mM to 100 mM, suitably from 10 mM to 80 mM, more suitably from 20 mM to 60 mM, e.g. may be about 10 mM. The concentration of glucose or fructose (when present) may range from 5 mM to 60 mM, 15 mM to 60 mM, suitably from 10 mM to 80 mM, more suitably from 20 mM to 40 mM, e.g. may be about 30 mM or about 60 mM. In one embodiment, the culture medium of step i) comprises at least glucose at a concentration ranging from 5 mM to 60 mM, 15 mM to 60 mM, from 10 mM to 80 mM, from 20 mM to 40 mM, is about 30 mM or is about 60 mM.

The inventors observed that controlling the osmolality of the culture medium in step i) proved to be advantageous for subsequent saponin production. A suitable osmolality range to be maintained during step i) and/or step ii) and/or step iii) (suitably during all 3 steps) of the method of the invention may be between 100 and 220 mOsm, more suitably, between 180 and 200 mOsm. Preferably, the osmolality is not higher than 200 mOsm.

Osmolality may be controlled by the source of carbon included in the culture medium. An osmolality between 180 and 200 mOsm may, for example, be achieved by targeting the glucose concentration in the culture medium at 60 mM. The level of glucose in the medium may be monitored and adjusted continuously or periodically. The inventors observed that classic media, such as MS medium, which typically contain about 80 mM of sucrose, led to peaks in osmolality higher than 200 mOsm. Accordingly, in some embodiments, the culture medium used in step i) of the method of the invention suitably contains between 2.5 mM to 40 mM of sucrose, more suitably between 5 mM and 20 mM of sucrose, e.g. 10 mM. Alternatively, the culture medium used in step i) contains no sucrose.

In some embodiments, the medium used in step i) comprises one or more hormone(s) selected from auxins and/or cytokinins. Suitably, the medium used in step i) comprises one or more hormones selected from NAA, 2,4-D and BA. More suitably, the medium used in step i) comprises at least 2,4-D. In some embodiments, the medium used in step i) comprises NAA and 2,4-D. Suitably, the concentrations of NAA and/or 2,4-D in the medium used in step i) may be from 0.2 mg/L to 0.8 mg/L, e.g. they may be about 0.4 mg/L, about 0.5 mg/L, or about 0.6 mg/L. In further embodiments, the medium used in step i) comprises NAA, 2,4-D and BA.

In some embodiments, the culture medium of step i) comprises further micronutrients and/or vitamins. Suitably, the further micronutrients are one or more of KI, H₃BO₃, MnSO₄, ZnSO₄, Na₂MoO₄, CuSO₄, CoCl₂, DeSO₄, or Na₂EDTA, and the vitamins are one or more of myo-inositol, nicotinic acid, pyrodixine-HCl, or thiamine-HCl.

In further embodiments, the culture medium in step i) comprises CaCl₂ and/or MgSO₄. Suitably, in the medium of step i), the concentration in CaCl₂ is from 1 to 5 mM, such as from 2 mM to 4 mM, for example about 3 mM. Suitably, in the medium of step i), the concentration in MgSO₄ is from 0.5 to 3 mM, such as from 1 mM to 2.5 mM, for example about 1.5 mM or about 2 mM.

An example of an appropriate culture medium to be used in step i) is Medium 4 or Medium 6, as described in the Example section (the composition of which being provided in Table 1 below). In some embodiments, the culture medium in step i) is Medium 4, or is Medium 6. The composition of the culture medium in step i) may require some adaptation to different plant cells, or cell lines. For example, the inventors observed that controlling the level of nutrients, such as glucose and phosphate, had an impact on saponin production. Advantageously, the target for glucose concentration in the culture medium in step i) may be between 40 mM and 70 mM, such as for example 60 mM and/or the target for phosphate concentration may be between 1 mM and 5 mM, such as for example 2.5 mM or 5 mM. Such levels of glucose and/or phosphate concentration are additionally advantageously targeted in the culture medium of step ii) and/or in the culture medium of step iii). It is within the ambit of the skilled person to adjust the concentration of the nutrients in the culture medium in any of step i), step ii) or step iii), by monitoring their concentration and consumption at any given time, e.g. by sampling the plant cells and measuring their concentration in the cell culture method by any method known in the art.

An easy way which the inventors used to assess whether any change in the culture conditions would subsequently impact saponin production is to look at the ability of plant cells cultivated in any given condition to produce foam, after nitrogen depletion, elicitation and mechanical disruption of the cells (e.g. as described below). Indeed, saponins are also known for their detergent activity. Accordingly, when the cells are producing saponins, some foam is visible after mechanical disruption. The occurrence and observation of such foam seems to correlate with saponin production, as confirmed by the subsequent measurement of saponins using suitable analytical methods (as described below). This provides the advantage of an easy read-out (visible to the naked eye) predictive of the final desired result. Sampling small volumes of suspension plant cells at different time points and in different conditions and then looking at foam production may be predictive of saponin production.

In the method of the invention, the plant cells may be cultured at any temperature known to be suitable for plant cell culture, and may be adjusted by the skilled person. For example, the temperature may range from 15° C. to 35° C., suitably ranging from 20° C. to 30° C., for example may be 25° C. In one embodiment, the method of the invention is operated at about 25° C.

For suspension cell culture, the plant cell culture may be agitated. Suitable ranges of agitation are from 30 rpm to 80 rpm, more suitably from 40 to 60 rpm, even more suitably is about 50 rpm. In one embodiment, the method of the invention is operated at about 50 rpm.

Step ii)—Depleting the Culture Medium from any Nitrogen Source

While looking for appropriate conditions for triggering saponin production, the inventors observed that, before triggering the saponin production by elicitation, the physiological state of the cells is important in order to achieve an optimal saponin production.

They observed that, prior to eliciting the cells to trigger saponin production, depleting the culture medium used in step i) from any source of nitrogen led to an appropriate physiological state of the cells, which resulted into an increased yield of saponins after subsequent elicitation (e.g. as described below). Without wishing to be bound to a theory, it is believed that nitrogen depletion changes the physiological state of the cells, making them more responsive to subsequent elicitation.

As described earlier, a nitrogen source has been reported to be important for the growth of plant cells cultured in vitro. However, surprisingly, the inventors observed that plant cells were also able to grow in the absence of a source of nitrogen, or in the presence of a reduced concentration of nitrogen source. Moreover, they observed, surprisingly, that culturing the plant cells in the absence of a source of nitrogen, or in the presence of a reduced concentration of nitrogen source (or letting the cells naturally consume the source of nitrogen present in the culture medium), prior to eliciting the cells, facilitated the subsequent production of saponins. This is the nitrogen depletion phase. While nitrogen depletion may not be sufficient to obtain saponin production, the inventors observed that it was a prerequisite to obtain a saponin production after elicitation.

Accordingly, the term “depleting the culture medium from any source of nitrogen”, in the context of the invention, means reducing the level of any source of nitrogen which has been included in the culture medium in step i) and maintaining the cells in such culture medium having a reduced level of nitrogen source.

Nitrogen depletion may be performed either (i) by letting the cells naturally consume the source of nitrogen included in the culture medium in step i) down to a residual level, with no further replenishment of the culture medium with any nitrogen source (or “natural depletion”); and/or (ii) by replacing the culture medium at the end of step i) with a culture medium which does not include any nitrogen source, or a culture medium including a reduced concentration of nitrogen source. It is within the ambit of the skilled person to monitor and measure the residual level of the nitrogen source in the culture, so as to determine the optimal duration of step ii), especially in case of natural depletion.

In the context of the present invention, the term “reduced concentration of nitrogen source” is to be understood by reference to the concentration of the nitrogen source used during step i), i.e. the reduced concentration in the replacing culture medium, or in the consumed culture medium, during step ii) is lower. Suitably, the reduced concentration of nitrogen source in the culture medium during step ii) is between 0 mM and 5 mM, or between 1.25 mM and 5 mM, may be about 1.25 mM, about 2.5 mM or about 5 mM, and the nitrogen source may be one or more of KNO₃, NH₄NO₃ and NH₄Cl. When the nitrogen source is NH₄Cl, the reduced concentration is suitably about 1.25 mM or about 2.5 mM.

Suitably, at the end of step ii) and prior to eliciting the cells, the concentration of nitrogen source in the culture medium is reduced by 2-fold, more suitably is reduced by 4-fold, and even more suitably is reduced by 8-fold, as compared with the concentration of nitrogen source included in the culture medium used in step i), for example, the culture media described in the previous section.

In some embodiments, at the end of step ii) and prior to eliciting the cells, the residual level of the source of nitrogen in the culture medium is less than 10 mM, less than 5 mM, less than 2.5 mM, or less than 1 mM. In further embodiments, at the end of step ii), the residual level of the source of nitrogen in the culture medium is between 1 mM and 2 mM.

In some embodiments, at the end of step ii) and prior to eliciting the cells, the residual level of nitrate in the culture medium is less than 5 mM, less than 2 mM, less than 1.5 mM, or less than 1 mM. In further embodiments, at the end of step ii), the residual level of nitrate in the culture medium is between 0.5 mM and 1.5 mM. In yet another embodiment, the level of nitrate in the culture medium is undetectable.

In some embodiments, at the end of step ii) and prior to eliciting the cells, the residual level of ammonium in the culture medium is less than 5 mM, less than 2 mM, less than 1.5 mM, less than 1 mM, or less than 0.5 mM. In further embodiments, at the end of step ii), the residual level of ammonium in the culture medium is between 0.5 mM and 1.5 mM. In yet another embodiment, the level of ammonium in the culture medium is undetectable.

The residual level of a source of nitrogen in a culture medium may be measured by any method known in the art. When the source of nitrogen contains nitrates, such as KNO₃ and/or NH₄NO₃, the residual level may be assessed by measuring the presence of NO₃ ions in the culture medium, for example, using a colorimetric-based assay relying upon an enzymatic reaction converting the nitrate into nitrite producing a colored compound which can be quantified using a spectrophotometer. When the source of nitrogen contains ammonium, such as NH₄Cl and/or NH₄NO₃, the residual level may be assessed by measuring the presence of NH₄⁺ ions in the culture medium, for example, using a colorimetric-based assay relying upon an enzymatic reaction converting the ammonium into a colored compound which can be quantified using a spectrophotometer.

It will be apparent to the skilled reader that the transitioning between step i) and step ii) of the method of the invention may be different according to how nitrogen depletion is performed, e.g. whether by replacing the culture medium in step i) or by natural depletion, i.e. letting the cells consume the nitrogen source included in the culture medium in step i), with no further replenishment of the culture medium with any nitrogen source, down to a residual level (see the diagrams at the top of Example 3 and in Experiment 10, of the Example section, respectively, providing a schematic view for illustrative purposes only).

When performing nitrogen depletion by replacing the culture medium in step i) with a culture medium containing no nitrogen source, or a culture medium including a reduced concentration of nitrogen source, the replacement marks the beginning of step ii). For example, if the plant cells are grown in suspension, the suspension may be centrifuged, and the culture medium is replaced with a culture medium containing no source of nitrogen, or a culture medium including a reduced concentration of nitrogen source (e.g. the reduced ranges or values described earlier). The inventors observed that the nitrogen source in the replacing culture medium in step ii) does not need to be completely absent to obtain saponin production during step iii). An example of an appropriate replacing culture medium to be used in step ii) is Medium 1, as described in the Example section (the composition of which being provided in Table 1 below). In some embodiments, the replacing culture medium in step ii) is Medium 1. The composition of the replacing culture medium in step ii) may alternatively be a variation of Medium 1 and be as described in the previous section relating to “step i)”, apart from the nitrogen source composition. The source of nitrogen in the replacing culture medium in step ii) is suitably as described earlier in the present section. In some embodiments, the replacing medium comprises one or more hormones, one or more nutrients, and/or one or more vitamins as described in the previous section relating to “step i)”. When the replacing culture medium includes no source of nitrogen, or does not include KNO₃, suitably, KCl is added as a source of potassium. Accordingly, in some embodiments, the replacing culture medium comprises KCl, e.g. at a concentration ranging from 5 to 30 mM, from 10 to 20 mM, for example is about 15 mM.

When performing nitrogen depletion by replacing the culture medium in step i) (e.g. by centrifugation) with a culture medium containing no nitrogen source, or a culture medium including a reduced concentration of nitrogen source, the cells cultured in step i) are centrifuged and a given cell biomass is resuspended directly into the replacing culture medium to obtain a desired PCV. Suitably, the PCV ranges between 10% and 40%, more suitably between 15% and 30%, and even more suitably, between 25% and 30%, e.g. is about 15%, about 20% or about 30%. Alternatively, cells are cultured in step i) until reaching such a PCV range or values, and the culture medium is simply replaced with the replacing culture medium including no nitrogen source, or including a reduced concentration of nitrogen source. In other words, in some embodiments, the PCV ranges or values at the end of step i) and at the beginning of step ii) are the same.

When performing nitrogen depletion by natural depletion, the last replenishment of the culture medium in step i) marks the beginning of step ii). The cells, during step ii), are then let naturally consume the nitrogen source included in the culture medium down to a residual level by culturing them in the same culture medium. Accordingly, the culture medium in step ii), in case of natural depletion, may be the culture medium as described previously in the section relating to “step i)”.

Accordingly, when performing nitrogen depletion by natural depletion, the PCV ranges or values at the beginning of step ii) may be the same as the ranges or values at the end of step i), that is, suitably, between 10% and 40%, more suitably between 15% and 30%, and even more suitably, between 25% and 30%, e.g. is about 15%, about 20% or about 30%. Alternatively, cells may be cultured in step i) until reaching a given PCV range or values, and then be diluted with fresh culture medium (last replenishment), so that the PCV range or values (after dilution) at the beginning of step ii) may be lower than at the end of step i).

Nitrogen depletion by replacing the culture medium in step i) with a culture medium which does not include any source of nitrogen source may have the advantage of shortening the duration of step ii). As a source of nitrogen is absent from the beginning of step ii), no time is required to reach a residual level (as compared with the natural depletion). However, especially at large scale, when using bioreactors, manipulating large volume bioreactors is more constrained and it is not always possible to easily remove the entirety of the culture medium. Moreover, when operating a process at large scale, there is a desire to reduce the number of operations required during the process. Natural depletion may then be advantageous in such a large scale setting.

Therefore, it will also be apparent to the skilled reader that the duration of step ii) may vary according to how nitrogen depletion is performed.

Suitable ranges for the duration of step ii) are from 1 to 9 days, from 2 to 7 days, from 5 to 7 days, more suitably from 3 to 6 days, for example is 4 days, 5 days or 7 days. These ranges are particularly suitable when nitrogen depletion is performed by replacing the culture medium in step i) with a culture medium which does not include any source of nitrogen source, or a culture medium including a reduced concentration of source of nitrogen. In some embodiments, after replacing the culture medium at the end of step i) with a culture medium which does not include any nitrogen source, or a culture medium including a reduced concentration of nitrogen source, the cells are maintained in the replacing culture from 1 to 9 days, from 2 to 7 days, from 5 to 7 days, from 3 to 6 days, for 4 days, for 5 days or for 7 days.

Alternatively, suitable ranges for the duration of step ii) are from 5 to 20 days, more suitably from 6 to 19 days, even more suitably, from 7 to 18 days, from 8 to 16 days, from 9 to 15 days, for example is 10 days, 11 days, 12 days, 13 days or 14 days. These ranges are particularly suitable when nitrogen depletion is performed by natural depletion. In some embodiments, after natural consumption of the nitrogen source down to a residual level, the cells are maintained in the consumed medium from 1 to 9 days, from 2 to 7 days, from 5 to 7 days, from 3 to 6 days, for 4 days, for 5 days, or for 7 days.

Determining an optimal duration of step ii), depending on the plant cell culture used and/or depending on the type of depletion employed is within the ambit of the skilled person. As described earlier, an easy way which the inventors used is to look at the ability of plant cells in any given condition after nitrogen depletion, elicitation and mechanical disruption of the cells (e.g. as described below) to produce foam. By sampling small volumes of plant cells at different time points after nitrogen depletion and subsequent elicitation (while testing different depletion conditions and/or different depletion durations), and then looking at foam production may be predictive of saponin production.

During the development of the method of the invention, the inventors occasionally observed a “starvation” of the cells in glucose and/or phosphate during the nitrogen depletion phase. This may negatively impact the level of saponin production after subsequent elicitation of the cells. Accordingly, during step ii), the level of glucose and/or phosphate in the culture medium may advantageously be monitored and replenished, as needed. For example, when the culture medium reaches a residual level of glucose of 15 mM or less, the culture medium may be fed with a solution of 60 mM glucose. Likewise, when the culture medium reaches a residual level of phosphate of 0.6 mM or less, the culture medium may be fed with a solution of 2.5 mM phosphate. Feeding may occur periodically or continuously. As the consumption rate of nutrients may vary from one cell line to another and from one depletion condition to another, it is within the ambit of the skilled person to determine, for each cell line and for each depletion condition the best conditions and modes of nutrients feeding, such as for example glucose or phosphate.

Step iii)—Eliciting the Production of the Saponins with at Least One Elicitor

Saponins are naturally occurring structurally and functionally diverse phytochemicals that are widely distributed in plants. They are generally considered to have important roles in defense of plants against pathogens, pests and herbivores due to their antimicrobial, antifungal, antiparasitic, and insecticidal properties (ref). Many plants synthesize and accumulate saponins during normal growth and development. The distribution of these natural products varies greatly among plant species, individual plants, organs and tissues, during development and maturation, and shows seasonal fluctuations. Some studies have suggested that variations in saponin distribution, composition and amounts in plants may be a reflection of varying needs for plant protection. In several plant species, the production of saponins is induced in response to biotic stress including herbivory and pathogen attack. Abiotic stress factors such as humidity, nutrient starvation, light and temperature can influence both the quality and quantity of saponin content. Increase in saponin levels in response to stress is often mediated by the transcriptional activation of biosynthetic genes through a complex signaling cascade involving the jasmonate and salicylate hormones. Hence, the biosynthesis of these molecules can be induced using elicitors and this feature has been exploited in several plant species to improve saponin yields (Yendo et al., 2010).

However, the inventors observed that elicitation may not be sufficient to consistently obtain a high yield of quillaic-based triterpenoid saponin and that a prior phase of nitrogen depletion (as described earlier) is needed to obtain high yield.

Accordingly, elicitation is performed after the plant cells were depleted from any nitrogen source, as described earlier.

Suitable elicitors for use in the invention are moncocarboxylic compound-type elicitors, such as 5-chlorosalicyclic acid, salicyclic acid, acetylsalicyclic acid, a methyl ester, e.g. methyl jasmonate (MeJa), or the chemically synthesized 2-HEJ. In one embodiment, the at least elicitor used in step iii) McJa.

In one embodiment, suitable ranges for the concentration of the at least one elicitor in step iii) ranges from 0.5 to 12 μM, more suitably from 1 to 8 μM, even more suitably from 2 to 6 μM, even more suitably from 3 to 5 μM. Other suitable concentrations are from 1 to 3 μM, such as for example about 2 μM. In some embodiments, the at least elicitor used in step iii) is MeJa used at a concentration ranging from 1 to 3 μM. In further embodiments, the at least elicitor used in step iii) is MeJa used at a concentration ranging from 2 to 6 μM, e.g. is about 2 μM, about 3 μM, or about 6 μM.

While the concentration of elicitors is typically referred to by reference to the volume of the culture medium, an alternative way to define the concentration, and used by the inventors, is by reference to the PCV %.

A suitable range of an elicitor, e.g. MeJa, for use in step iii) is from 0.5 to 12 μM/PCV %, more suitably, from 1 to 8 μM/PCV %, even more suitably from 2 to 6 μM/PCV %, even more suitably from 3 to 5 μM/PCV %. A particularly suitable range of MeJa is from 1 to 3 μM/PCV %, e.g. 2 μM/PCV %. In some embodiments, the at least elicitor used in step iii) is MeJa used at a concentration ranging from 1 to 3 μM/PCV %. In further embodiments, the at least elicitor used in step iii) is MeJa used at a concentration ranging from 2 to 6 μM/PCV %, e.g. is 2 μM/PCV %, 3 μM/PCV % or 6 μM/PCV %

Elicitors may be added directly to the culture medium. Accordingly, elicitors may be added directly to the culture medium at the end of step ii). This marks the beginning of step iii). Elicitors may be added once, or may be further added, for example, every other day over the duration of step iii). Alternatively, the culture medium may be replaced with a culture medium including the elicitor(s) at the end of step ii), marking the beginning of step iii). Such culture medium containing the elicitors may advantageously additionally contain nutrients which may have been consumed, such as the carbon source or the phosphate source, but does not contain any source of nitrogen.

It is within the ambit of the skilled person to monitor (e.g. by sampling) the consumption level of nutrients along the different phases of the method of the invention, and replenish the nutrients which may have been consumed.

Suitably, the elicitation may take place between 1 to 14 days, e.g. 10 days, more suitably between 2 to 10 days, even more suitably, between 3 to 8 days, e.g. 7 days, even more suitably, between 4 to 6 days, e.g. 5 days. In other words, plant cells may be harvested from 1 to 14 days, e.g. 10 days, from 2 to 10 days, from 3 to 8 days, e.g. 7 days, from 4 to 6 days, e.g. 5 days after addition of the first shot of the at least one elicitor, before proceeding with step iv) and recovering the saponins produced.

Determining the optimal concentration of the elicitor(s) and the optimal duration of the elicitation, for a given cell line, is within the ambit of the skilled person. This may be assessed, e.g. by conducting a time point experiment with different concentrations of elicitor(s), while measuring the level of saponin (as described below). The skilled person would thus be able to determine the best conditions for any given plant cell culture. Similarly as described earlier, the occurrence and observation of foam in the plant cells, after mechanical disruption, may be looked at by sampling as a prediction of the level of saponin production achieved.

As the elements/nutrients contained in the culture medium may have been consumed and/or may be consumed when eliciting, in particular the carbon sources and the phosphate source, it may therefore be necessary to restore the composition of the culture medium at the time of the elicitation and/or during the elicitation. This may be done by replenishing the culture medium with, e.g. glucose and phosphate, continuously or periodically as appropriate. As described earlier, during the development of the method of the invention, the inventors occasionally observed a “starvation” of the cells in glucose and/or phosphate during the elicitation phase. This may negatively impact the level of saponin production. Accordingly, during step iii), the level of glucose and/or phosphate in the culture medium may advantageously be monitored and replenished, as needed. For example, when the culture medium reaches a residual level of glucose of 15 mM or less, the culture medium may be fed with a solution of 60 mM glucose. Feeding may occur periodically or continuously. As the consumption rate of nutrients may vary from one cell line to another and from one elicitation condition to another, it is within the ambit of the skilled person to determine, for each cell line and for each elicitation condition the best conditions and modes of nutrients feeding, such as for example glucose or phosphate.

Step iv)—Recovering the Saponins

At any appropriate time after elicitation, saponins are recovered. Saponins may be recovered by any methods known in the art, such as extraction using a non-aqueous polar solvent, extraction using an acid medium or a basic medium, or recovery by resin absorption, or extraction by mechanically disrupting the plant cells, such as by ball milling or sonication. Alternatively, saponins may be extracted by freezing the cell pellet (resulting in cell lysis) obtained after centrifugation of the cell culture. Suitably, the cell pellet is frozen at −20° C., and more suitably at −70° C., e.g. at least for 24 hours.

Any method known in the art to analyze and quantify the saponin content of any composition or any extract may be used, e.g. UPLC-UV-MS absorbance at 214 nm. Alternatively, the saponin content may be determined by HPLC-ELSD (Evaporative Light Scattering Detector), or by LCMS-MS. The use of appropriate standards for the saponins of interest to be looked at allows to quantify the respective saponin content in extracts of plant cells of the invention. For example, suitable standards may be saponin fractions isolated from the crude bark extract of Quillaja Saponaria trees, e.g. the fraction QS-21 (e.g. as described and reported in Kensil et al. 1991 or in WO 19/10692), the fraction QS-18, the fraction QS-17, and the fraction QS-7 (e.g. as described and reported in Kensil et al. 1991).

### Adjuvant Formulation

The saponins produced according to the methods of the invention may suitably be used as adjuvants, for example to be included in a vaccine. Any adjuvant formulation type known in the art may be used. For example, one or more saponins produced, such as QS-21 saponins, may be formulated into liposomes (see e.g. WO 2019/106192 or WO 2013/041572).

### Saponins

As described above, the saponins of the invention are quillaic acid-based triterpenoid glycosides. Using a crude bark extract of Quillaja saponaria trees as a reference, such saponins are traditionally known and regrouped as fractions, such as e.g. QS-17, QS-7, QS-21, or QS-18 fractions. Said fractions usually, each, contain a mixture of structurally-related saponin species (see e.g. Kensil et al. 1991), which saponin species being detailed below and grouped by “family”. QS-21 and QS-7 are saponin families of particular interest due to their immuno-stimulant activity.

In some embodiments, the saponins produced by the methods of the invention are one or more saponin species from the QS-7 saponin family, the QS-17 saponin family, the QS-18 saponin family and/or the QS-21 saponin family.

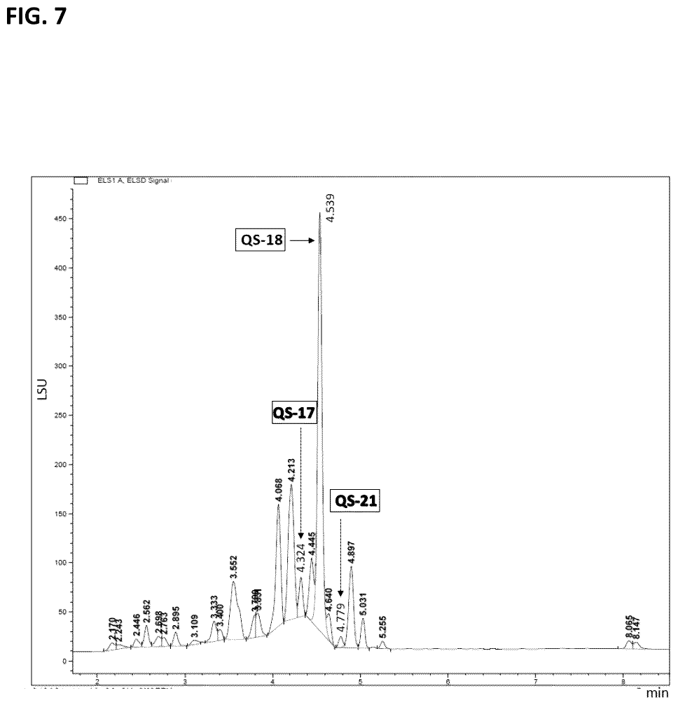

The inventors observed that all quillaic acid-based triterpenoid glycosides naturally synthesized in plant cells were indifferently produced when using the method of the invention, even though some variability in the producing capacity has been observed between different plant cells, or cell lines, which may reflect some inherent ability of given plant cells, or given cell lines, to synthesize saponins. Also, in line with what has been observed in the bark from Quillaja saponaria trees, the inventors observed that, very often, the most abundant saponins produced by the plant cells or cell lines of the invention are saponins from the QS-18 saponin family (as further detailed below) (see FIG. 7), while the respective proportions of each family may vary from one cell line to another (as shown in the Examples). The data disclosed in the Examples herein mainly provide QS-18 volumetric productivity and QS-21 volumetric productivity. However, QS-17 volumetric productivity has also been analysed, and was observed to similarly increase when using the method of the invention (see FIG. 7 and data not shown).

Suspension cell lines of plant cells naturally synthesizing quillaic acid-based triterpenoid saponins capable of producing such saponins at a volumetric productivity of at least 5 mg/L, at least 10 mg/L, at least 20 mg/L, at least 40 mg/L or at least 50 mg/L also form an object of the invention. In specific embodiments, the saponins produced by the suspension cell lines of plant cells of the invention are one or more saponin species from the QS-7 saponin family, the QS-17 saponin family, the QS-18 saponin family and/or the QS-21 saponin family

Each saponin family has one or more common structural features which characterise the family relative to other families. Individual species within each family also display certain structural features which characterise the species relative to other species of the family, including: xylose or rhamnose chemotype—the presence of a xylose or rhamnose residue in the trisaccharide at the C3 position of quillaic acid; A or B isomers—A having the acyl chain linked through the 4-position of the D-fucose at the C28 position of quillaic acid, B having the acyl chain linked through the 3-position of the D-fucose; V1 and V2—the presence of a terminal apiose or xylose residue, respectively, in the saccharide at the C28 position of quillaic acid (in other species of a family this terminal residue may also be absent).

Those skilled in the art will also recognise that the structures described herein contain ionisable groups and under appropriate circumstances may exist in dissociated forms or as salts. Structures are generally shown with the glucuronate moiety in ionised form and the indicated molecular weight is calculated directly from the ion shown (corresponding to the monoisotopic m/z observed with negative mode electrospray mass spectrometry), however, all non-dissociated, dissociated and salt forms are intended to be encompassed by the recited definitions. Salts are desirably pharmaceutically acceptable, although non-pharmaceutically acceptable salts can nevertheless be useful during manufacture of pharmaceuticals or for non-pharmaceutical uses.

### Group I—QS-7 Saponin Family

The term “QS-7 saponin family” (triterpenoid glycosides having an acetyl group linked through the 4-position of the D-fucose at the C28 position of quillaic acid) as used herein means (i) the xylose chemotype QS-7 species of a monoisotopic molecular weight (m/z) of 1862 (“QS-7 1862”) with negative mode electrospray mass spectrometry (which may exist as V1 apiose and V2 xylose isomers):

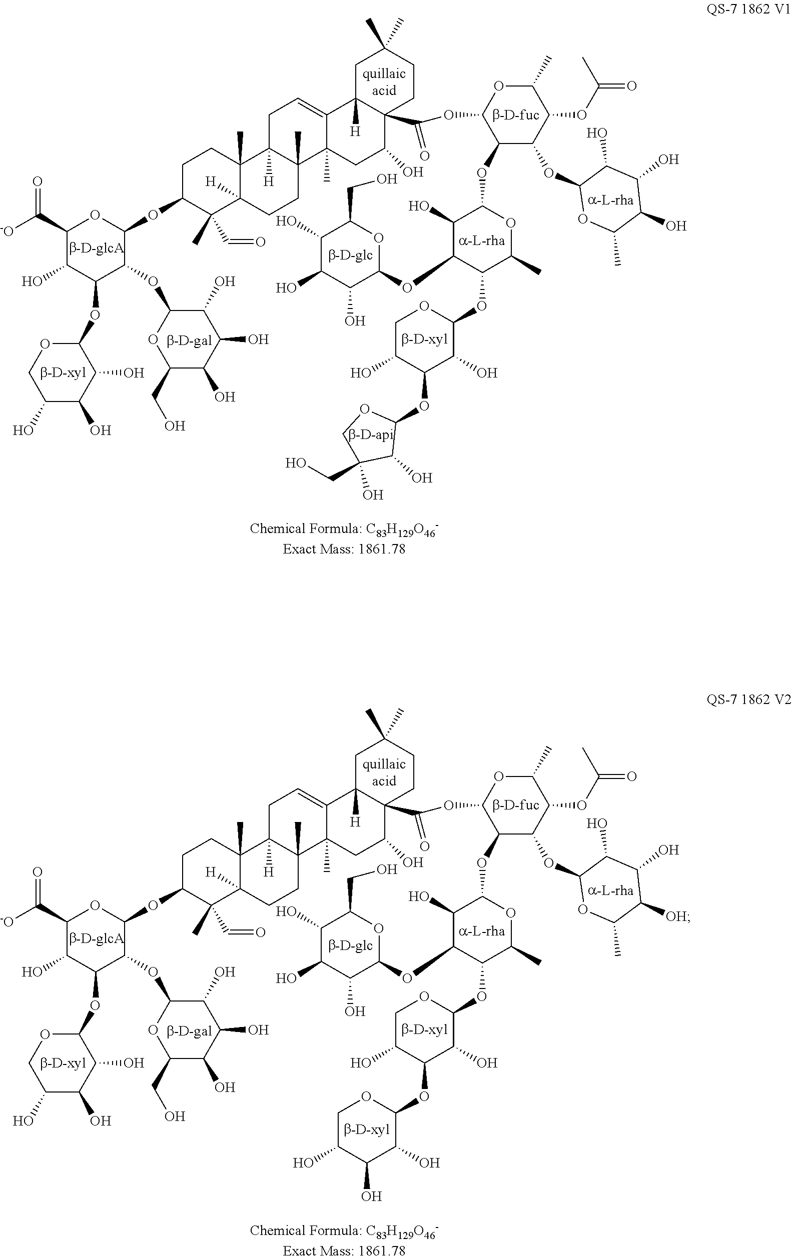

(ii) the xylose chemotype QS-7 species of a monoisotopic molecular weight (m/z) of 1730, 1700, 1568, 1554 and 1716 with negative mode electrospray mass spectrometry:

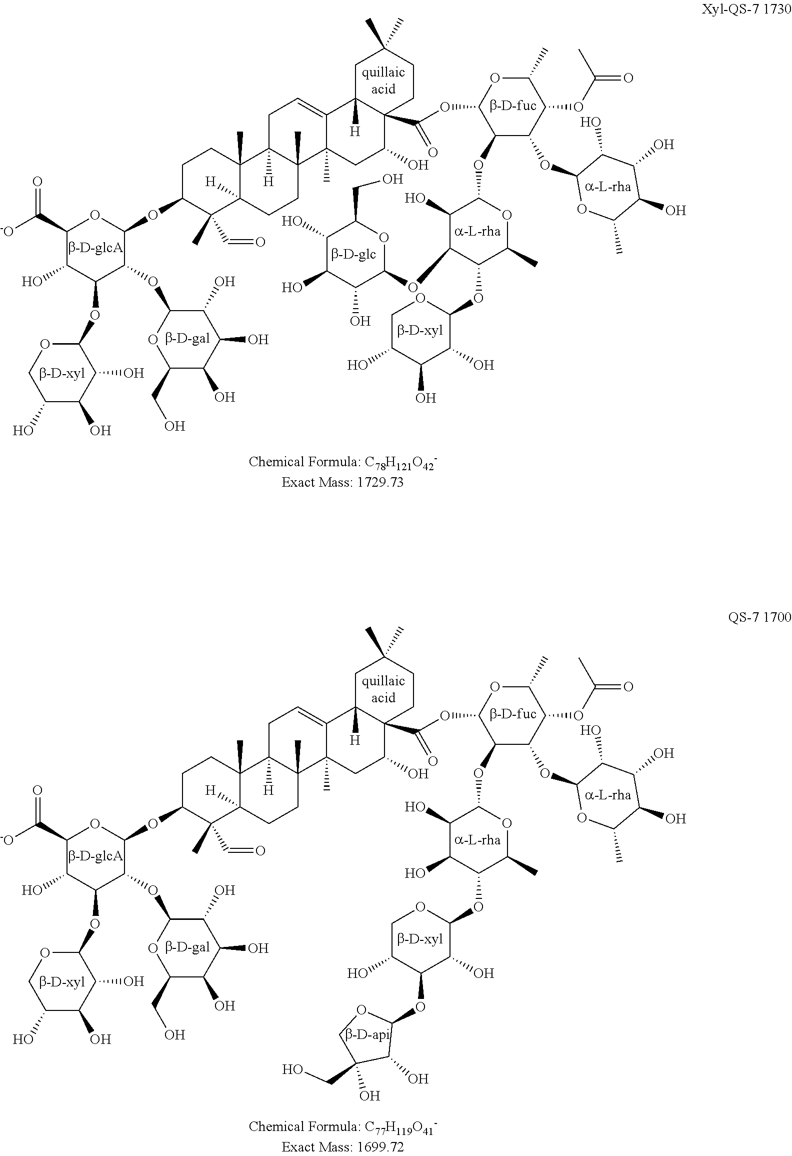

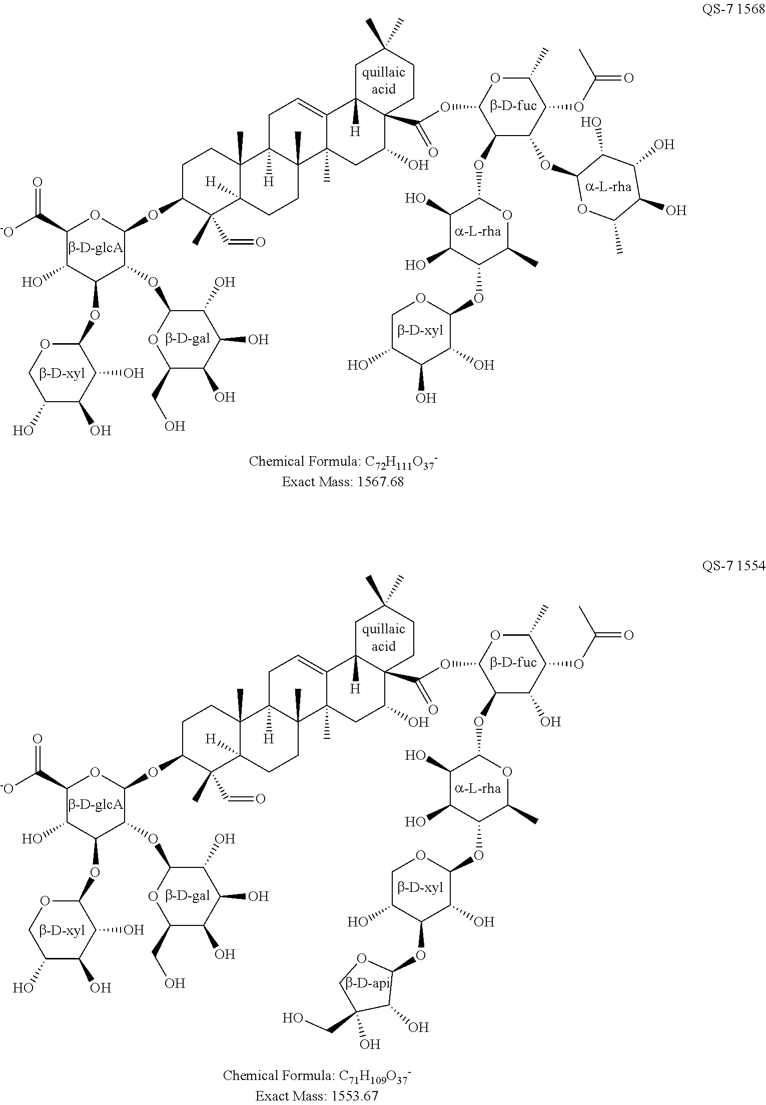

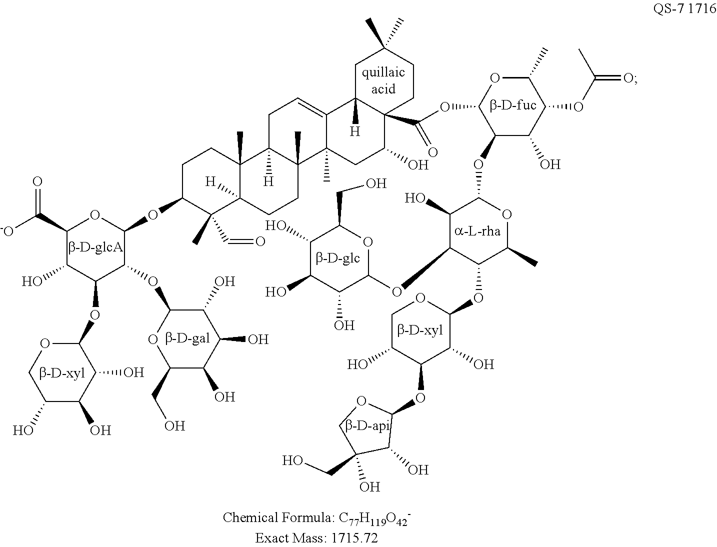

(iii) the rhamnose chemotype QS-7 species of a monoisotopic molecular weight (m/z) of 1876 (“QS-7 1876”) with negative mode electrospray mass spectrometry (which may exist as V1 apiose and V2 xylose isomers):

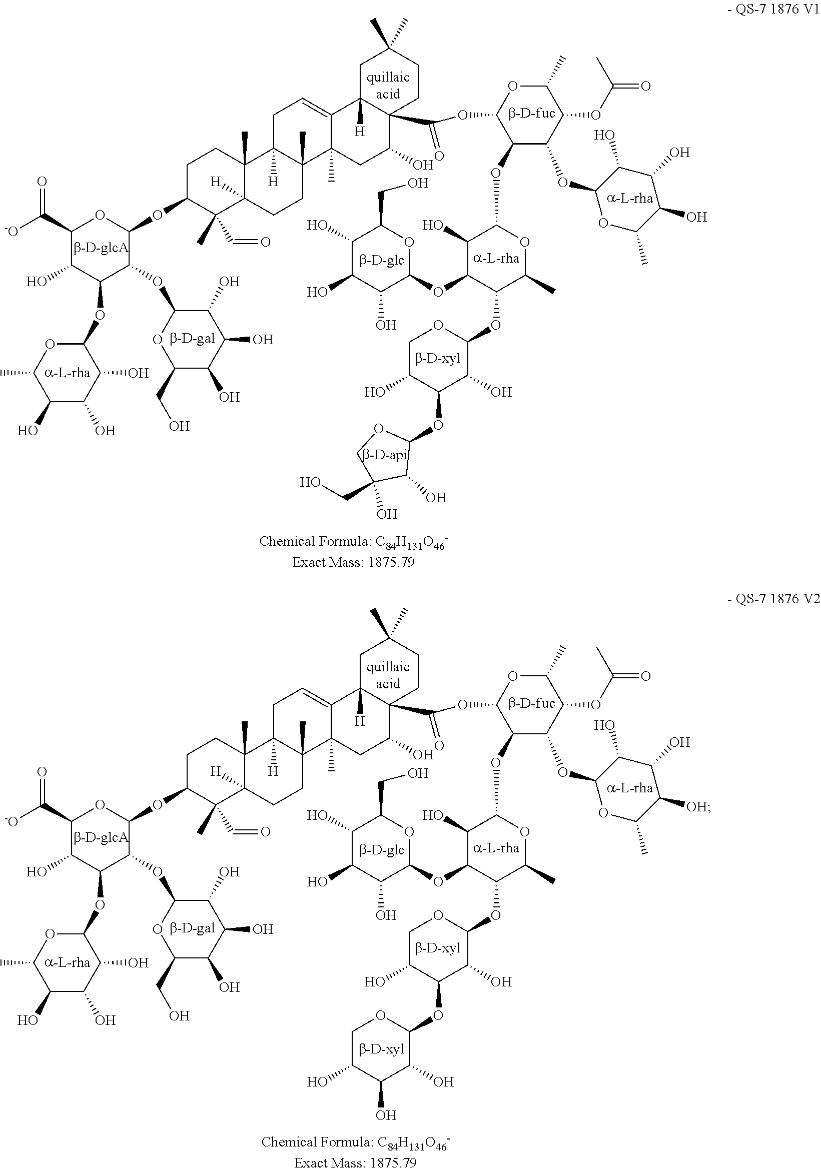

iv) the rhamnose chemotype QS-7 species of a monoisotopic molecular weight (m/z) of 1714 (“QS-7 1714”) with negative mode electrospray mass spectrometry (which may exist as V1 apiose and V2 xylose isomers):

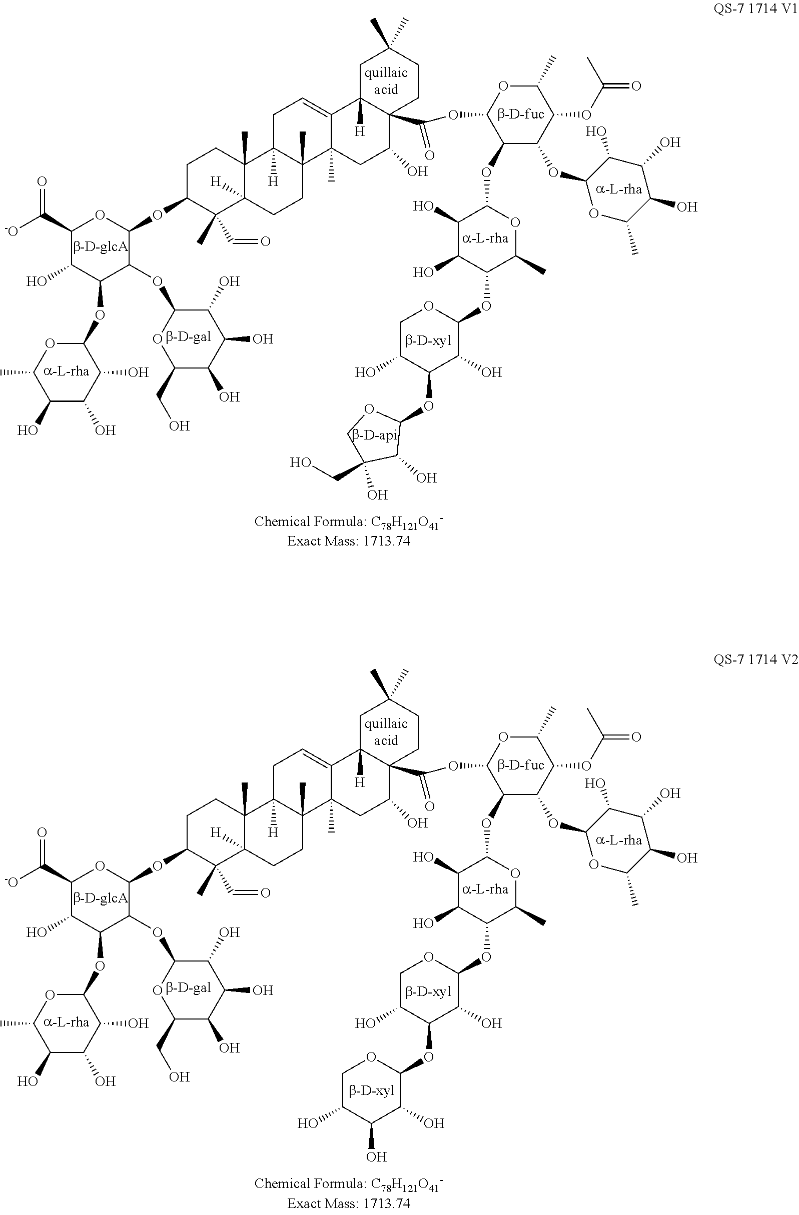

(v) the rhamnose chemotype QS-7 species having a monoisotopic molecular weight (m/z) of 1568 (“QS-7 1568”) with negative mode electrospray mass spectrometry (which may exist as V1 apiose and V2 xylose isomers):

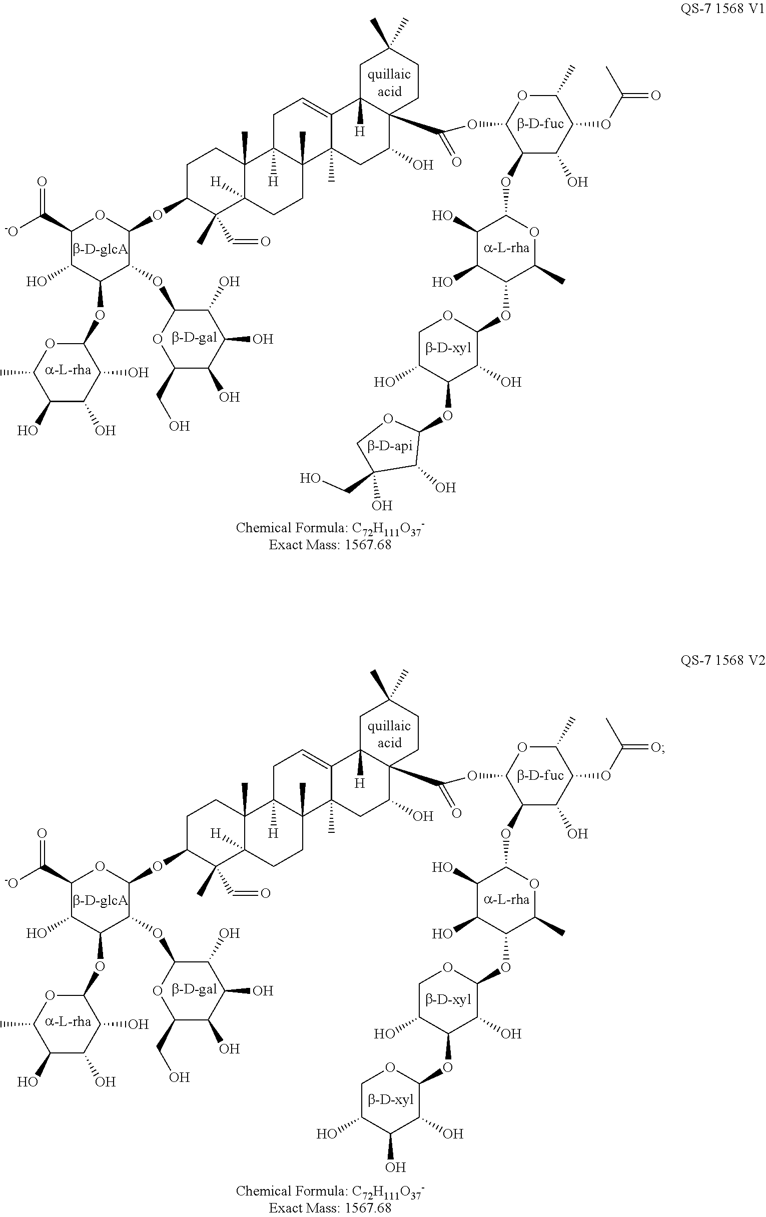

(vi) the rhamnose chemotype QS-7 species having a monoisotopic molecular weight (m/z) of 1730 with negative mode electrospray mass spectrometry:

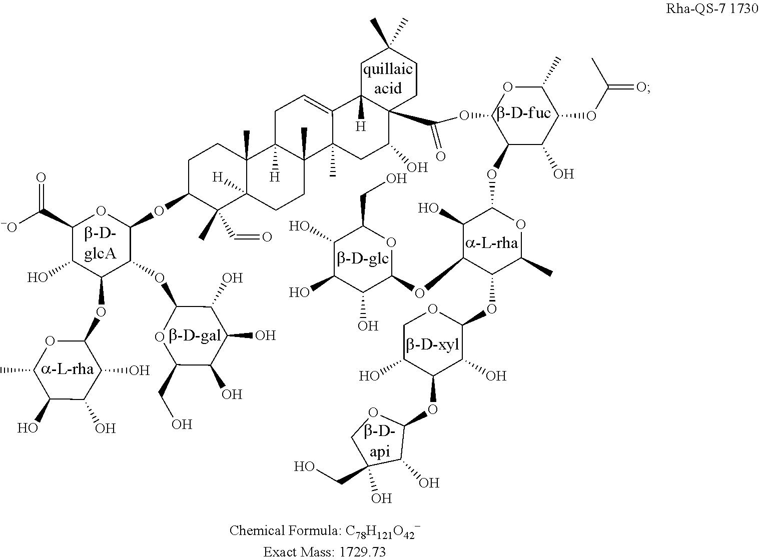

and (vii) the rhamnose chemotype QS-7 species having a monoisotopic molecular weight (m/z) of 1582 with negative mode electrospray mass spectrometry:

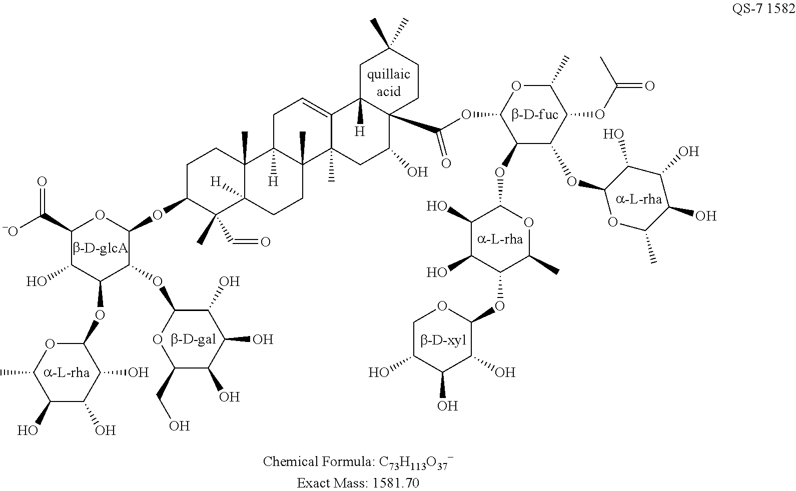

In one embodiment, the saponins produced by the method of the invention are one or more of the above QS-7 saponin species from the QS-7 saponin family.

In a further embodiment, the invention provides suspension cells lines of plant cells capable of producing one or more of the above QS-7 saponin species from the QS-7 saponin family.

### Group II—QS-18 Saponin Family

The term “QS-18 saponin family” (triterpenoid glycosides having beta-O-glucopyranosylation at the C3 position of the rhamnose residue of the saccharide at the C28 position of quillaic acid) as used herein means (i) the xylose chemotype QS-18 species having a monoisotopic molecular weight (m/z) of 2150 (“QS-18 2150”) with negative mode electrospray mass spectrometry (which may exist as A and B isomers, and V1 apiose and V2 xylose isomers):

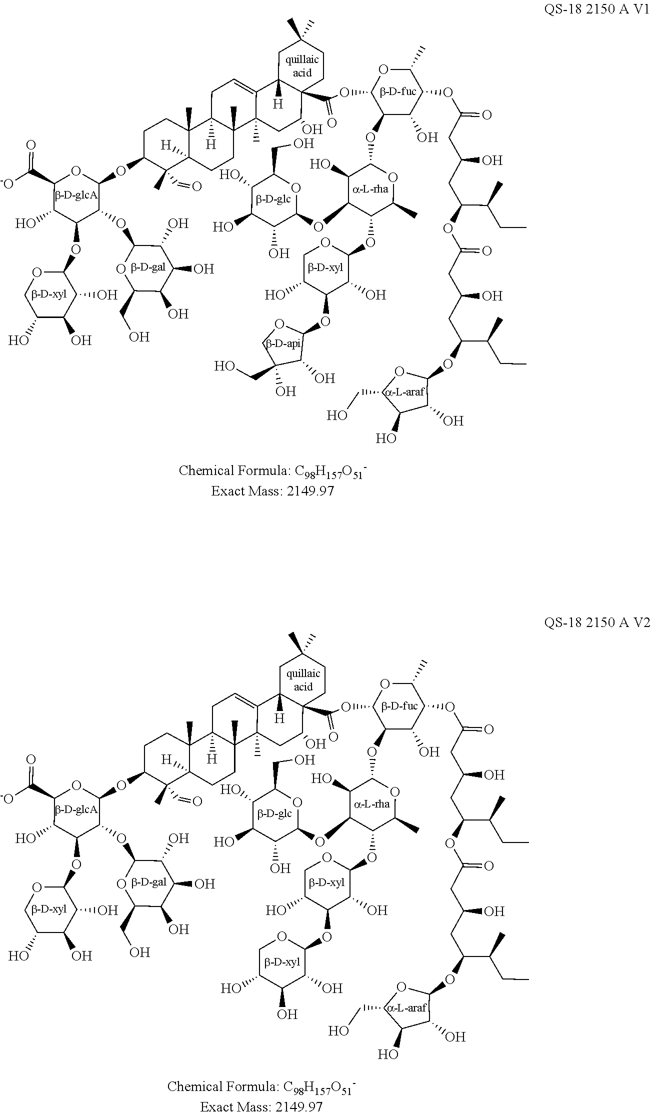

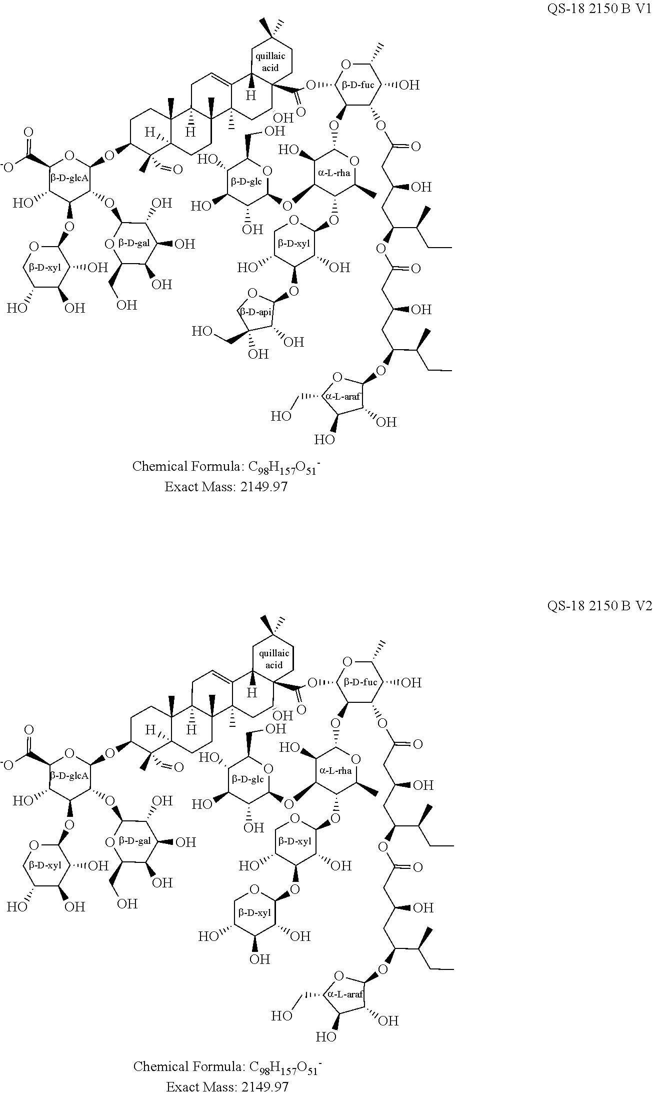

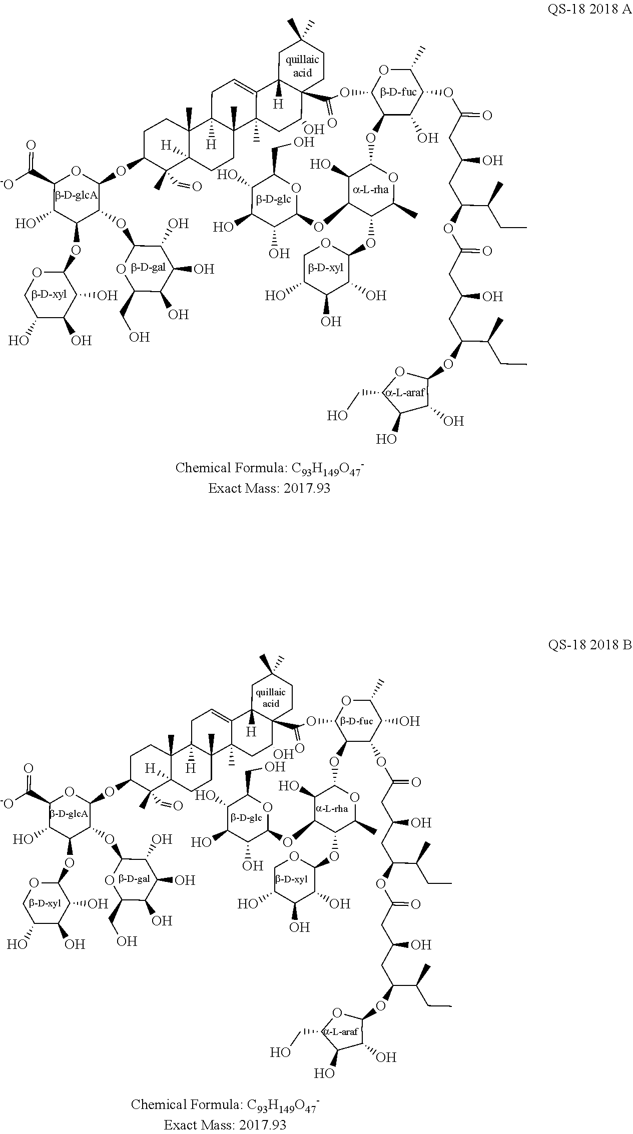

(ii) the xylose chemotype QS-18 species having a monoisotopic molecular weight (m/z) of 2018 (“QM-18 2018”) with negative mode electrospray mass spectrometry (which may exist as A and B isomers):

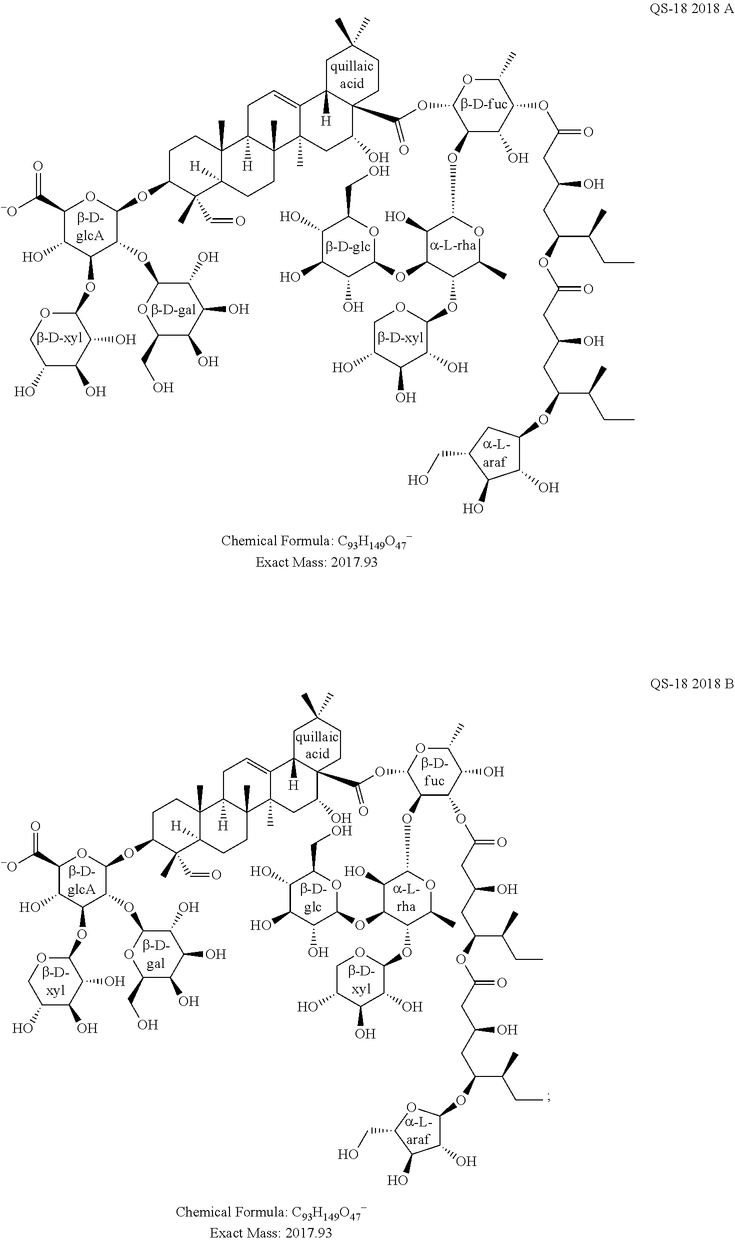

(iii) the rhamnose chemotype QS-18 species having a monoisotopic molecular weight (m/z) of 2164 (“QS-18 2164”) with negative mode electrospray mass spectrometry (which may exist as A and B isomers, and V1 apiose and V2 xylose isomers):

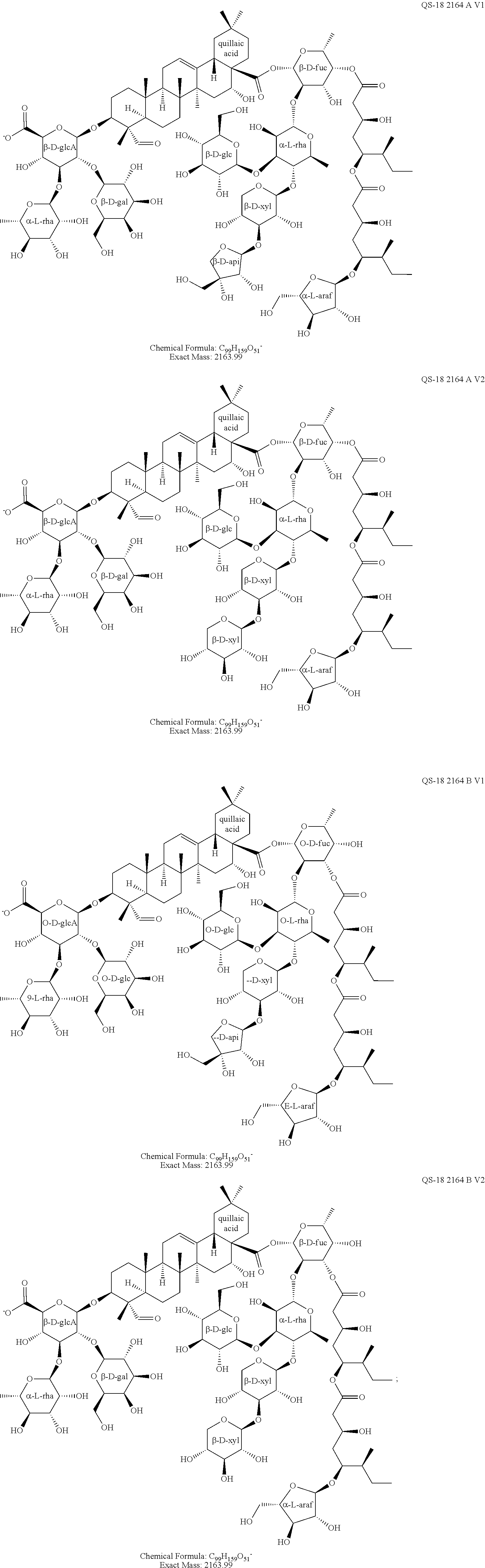

and (iv) the rhamnose chemotype QS-18 species having a monoisotopic molecular weight (m/z) of 2032 (“QS-18 2032”) with negative mode electrospray mass spectrometry (which may exist as A and B isomers):

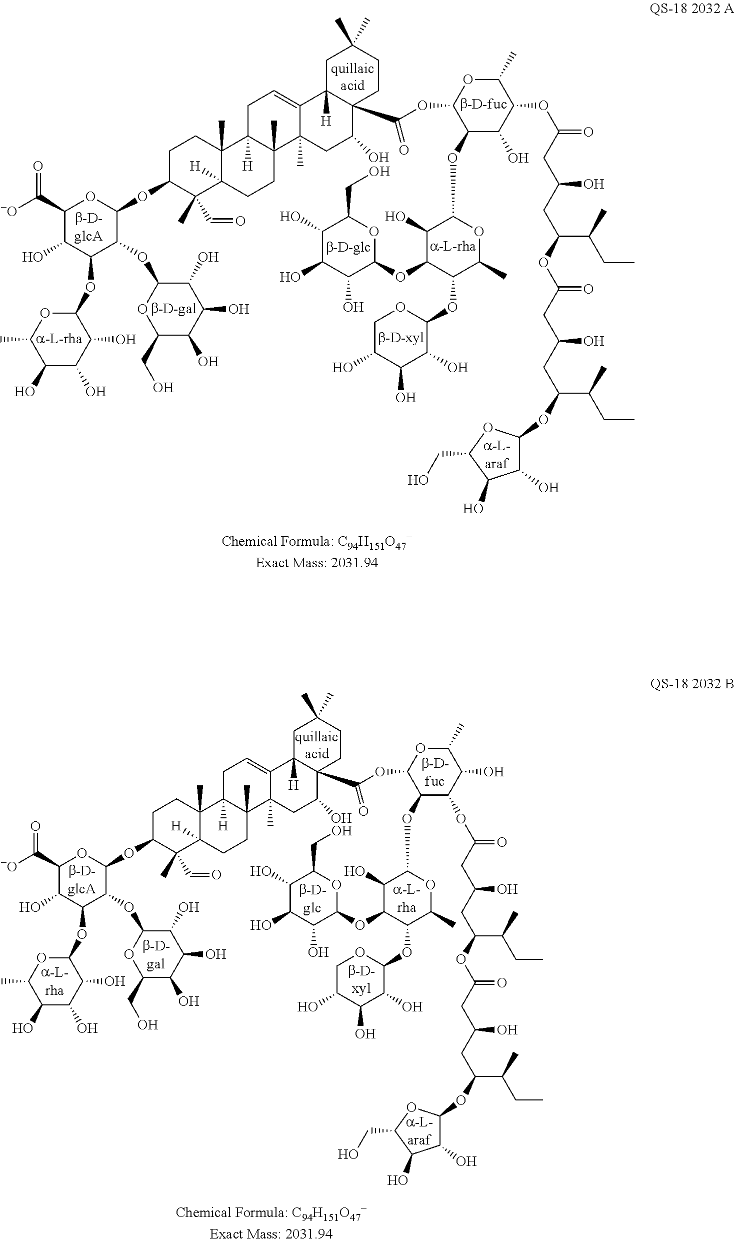

In some embodiments, the saponins produced by the methods of the invention are one or more of the above QS-18 saponin species from the QS-18 saponin family. In specific embodiments, the saponins produced by the methods of the invention are one or more from QS-18 2150 A V1, QS-18 2150 A V2, QS-18 2150 B V1 and QS-18 2150 B V2.

In a further embodiment, the invention provides a suspension cell line of plant cells capable of producing one or more of the above QS-18 saponin species from the QS-18 saponin family. In a specific embodiment, the saponins produced by the suspension cell line of plant cells of the invention are one or more from QS-18 2150 A V1, QS-18 2150 A V2, QS-18 2150 B V1 and QS-18 2150 B V2.

### Group III—QS-17 Saponin Family

The term “QS-17 saponin family” (triterpenoid glycosides having beta-O-glucopyranosylation at the C3 position of the rhamnose residue in the saccharide at the C28 position of quillaic acid and alpha-O-rhamnosylation at the C2 position of the arabinofuranose moiety of the acyl chain linked to the fucose residue in the saccharide at the C28 position of quillaic acid) as used herein means (i) the xylose chemotype QS-17 species having a monoisotopic molecular weight (m/z) of 2296 (“QS-17 2296”) with negative mode electrospray mass spectrometry (which may exist as A and B isomers, and V1 apiose and V2 xylose isomers):

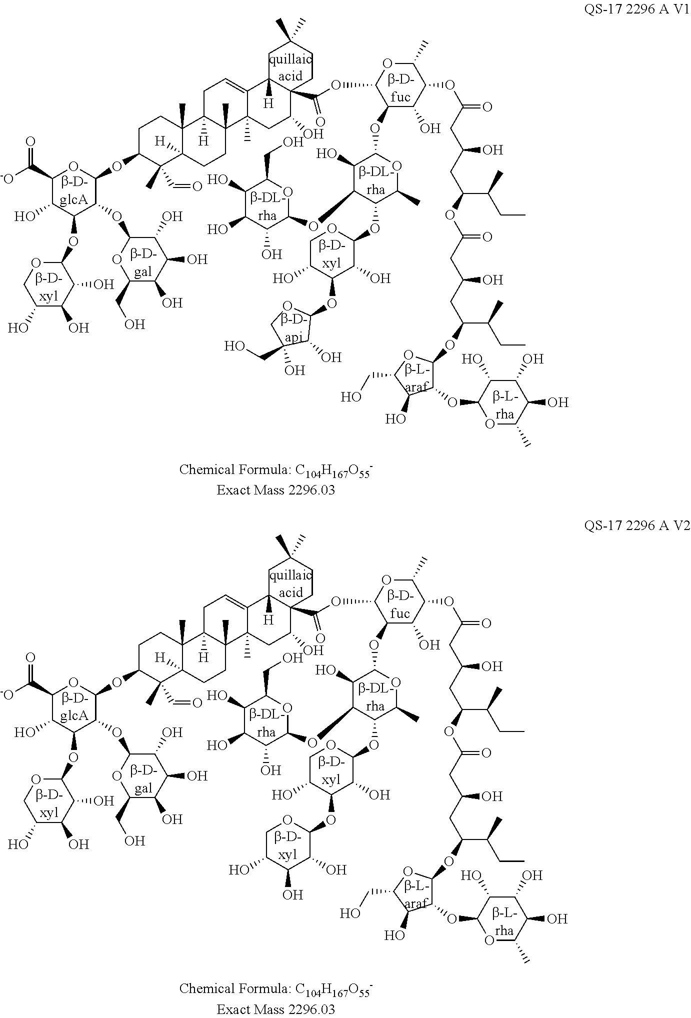

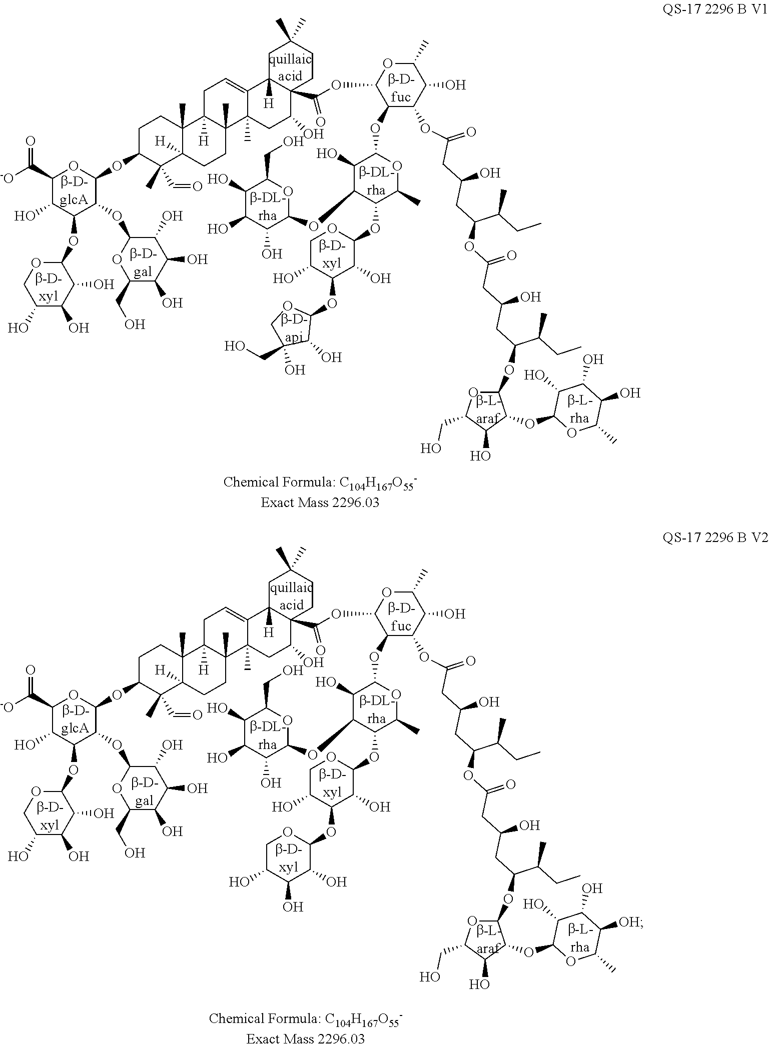

(ii) the xylose chemotype QS-17 species having a monoisotopic molecular weight (m/z) of 2134 (“QS-17 2134”) with negative mode electrospray mass spectrometry (which may exist as A and B isomers, and V1 apiose and V2 xylose isomers):

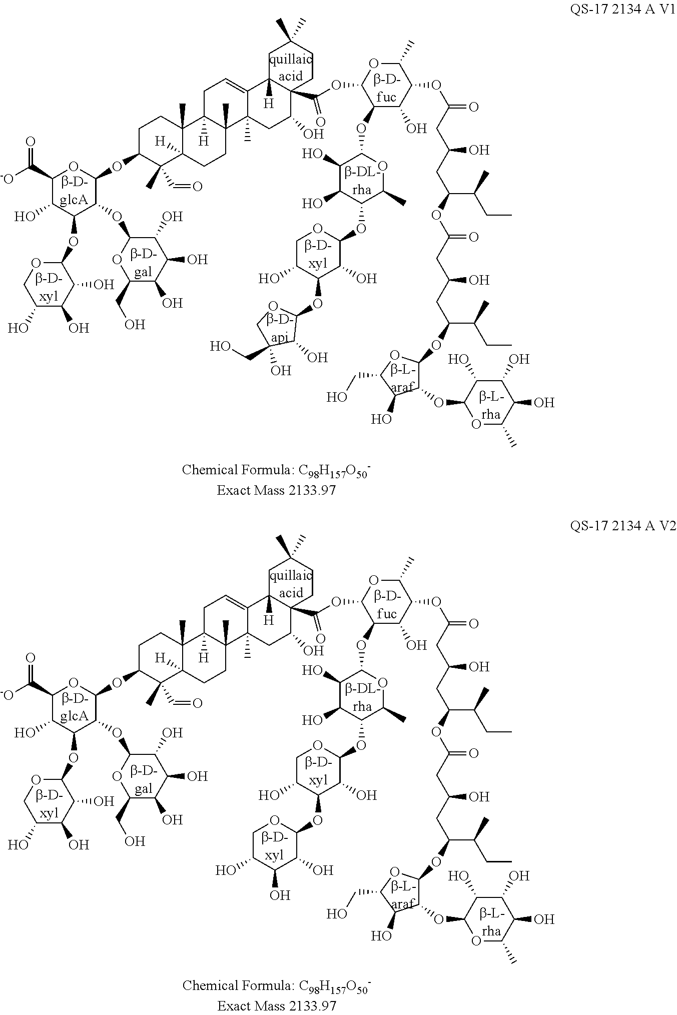

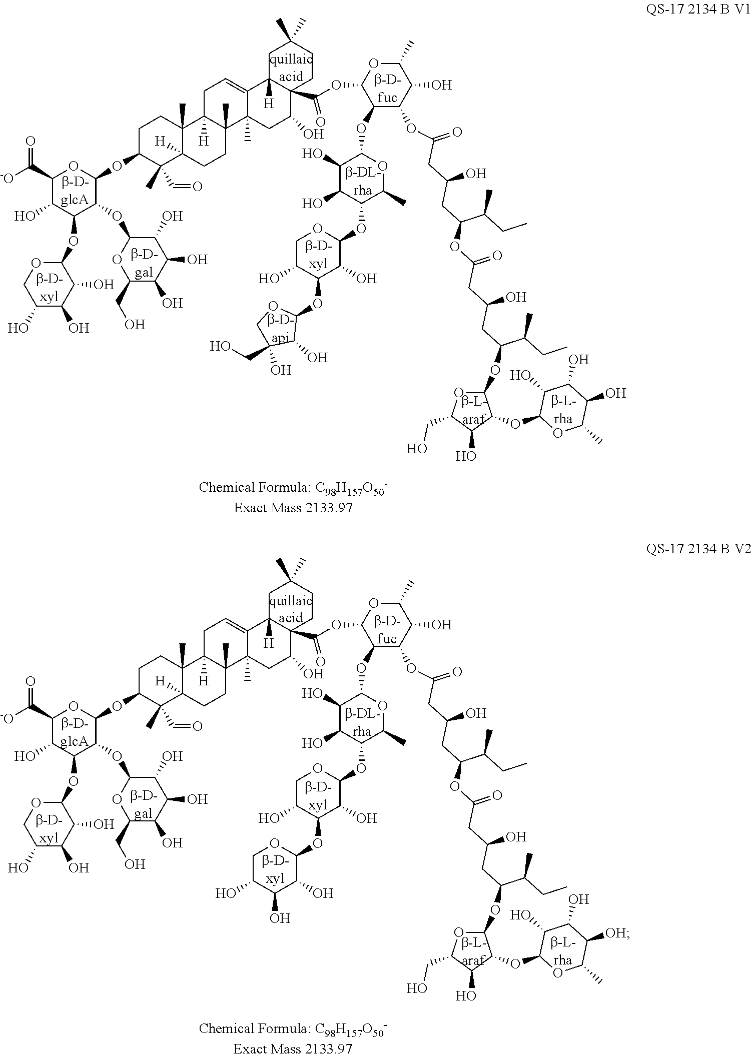

(iii) the xylose chemotype QS-17 having a monoisotopic molecular weight (m/z) of 2164 (“QS-17 2164”) with negative mode electrospray mass spectrometry (which may exist as A and B isomers):

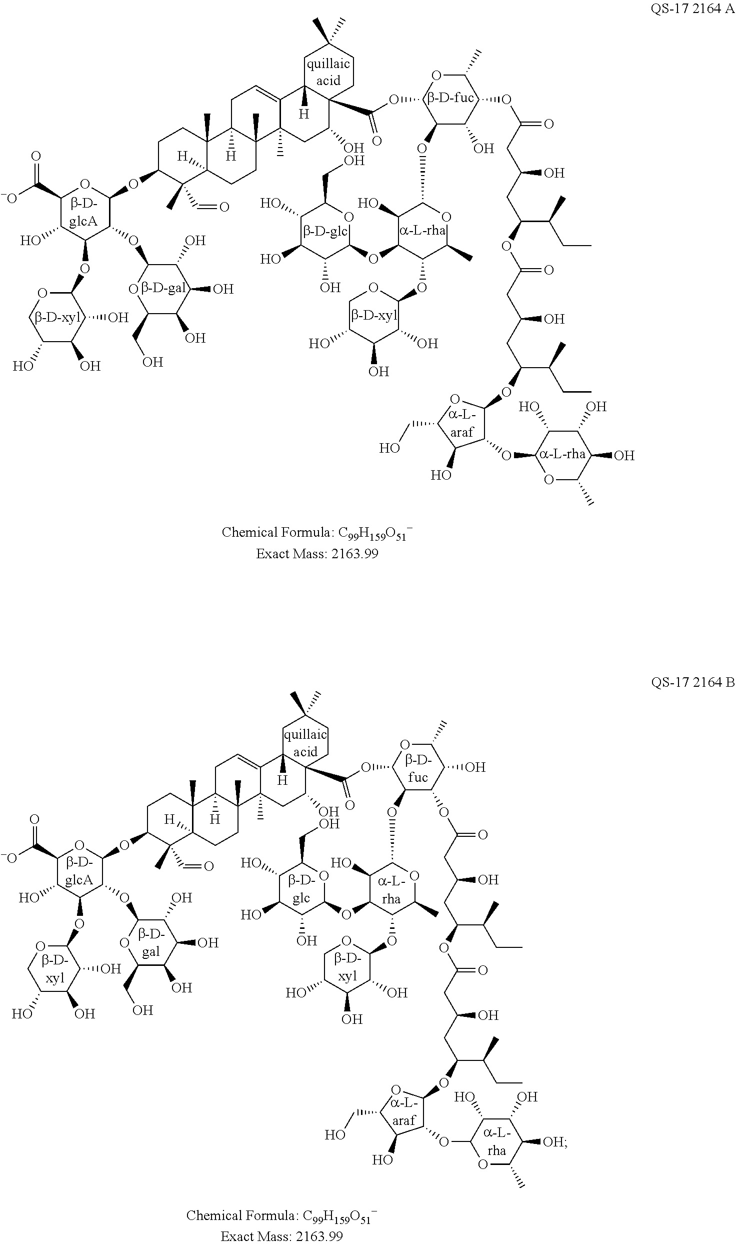

(iv) the rhamnose chemotype QS-17 species having a monoisotopic molecular weight (m/z) of 2310 (“QS-17 2310”) with negative mode electrospray mass spectrometry (which may exist as A and B isomers, and V1 apiose and V2 xylose isomers):

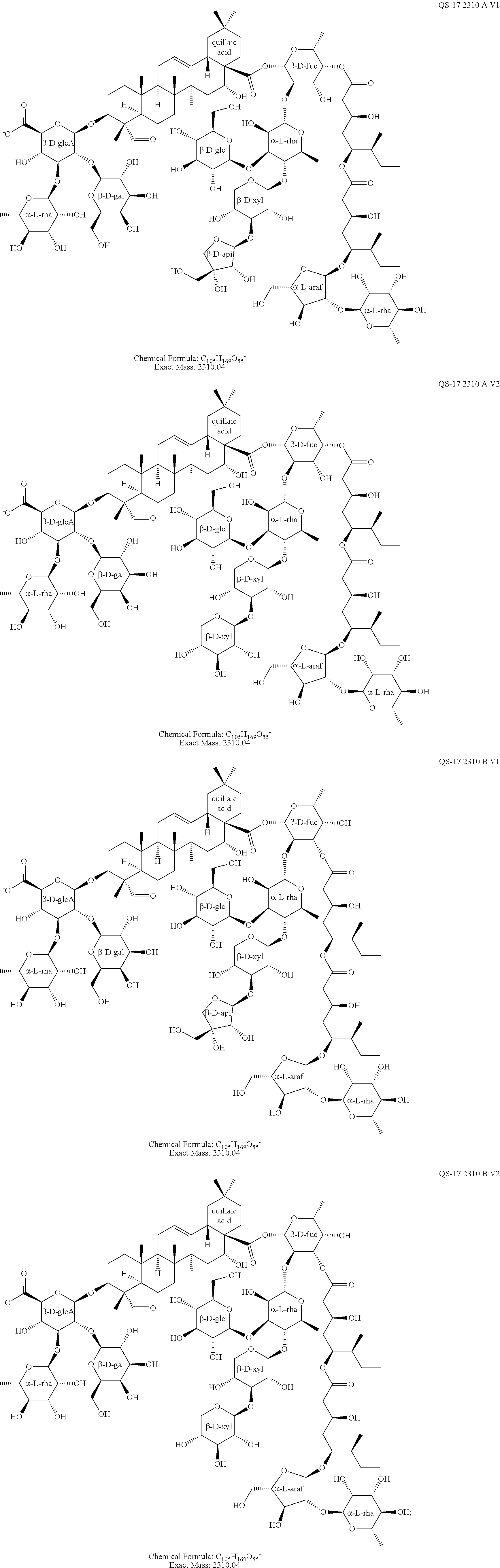

and (v) the rhamnose chemotype QS-17 species having a monoisotopic molecular weight (m/z) of 2148 (“QS-17 2148”) with negative mode electrospray mass spectrometry (which may exist as A and B isomers, and V1 apiose and V2 xylose isomers):

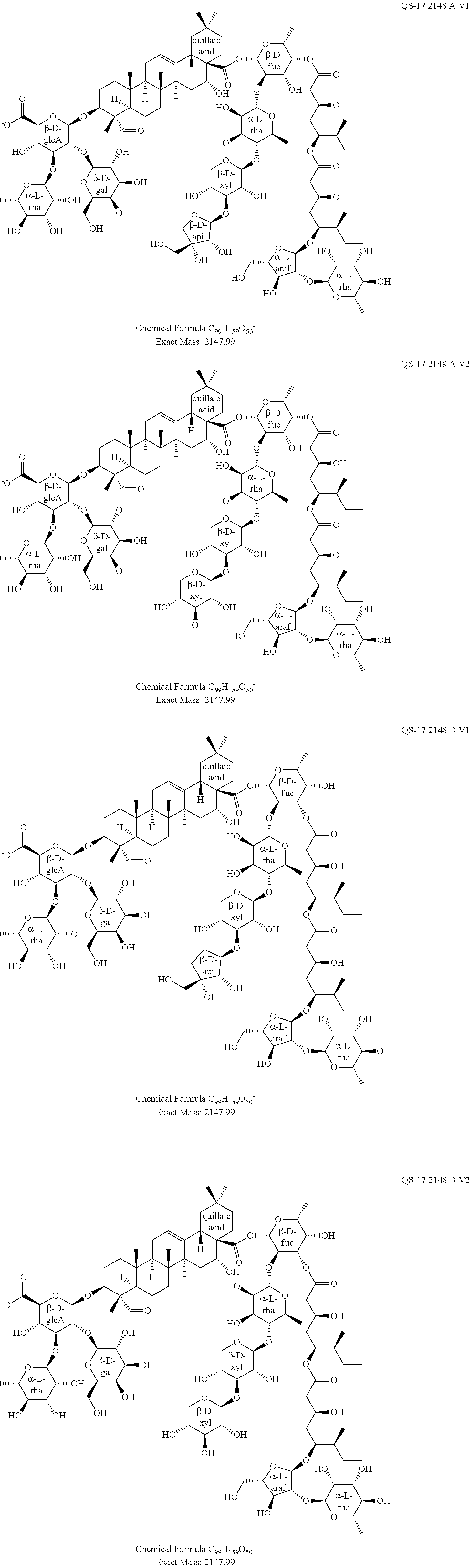

In one embodiment, the saponins produced by the method of the invention are one or more of the above QS-17 saponin species from the QS-17 saponin family. In a further embodiment, the invention provides a suspension cell line of plant cells plant capable of producing one or more of the above QS-17 saponin species from the QS-17 saponin family.

### Group IV—QS-21 Saponin Family

The term “QS-21 saponin family” (triterpenoid glycosides having an acyl chain linked at the fucose residue in the saccharide at the C28 position of the quillaic acid core which is terminated by an arabinofuranose residue) as used herein means (i) the xylose chemotype QS-21 species having a monoisotopic molecular weight (m/z) of 1988 (“QS-21 1988”) with negative mode electrospray mass spectrometry (which may exist as A and B isomers, and V1 apiose and V2 xylose isomers):

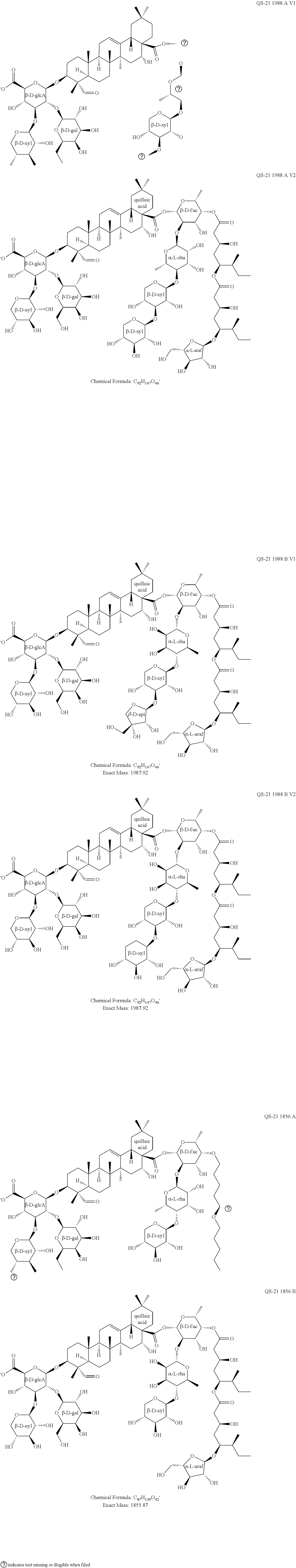

(ii) the xylose chemotype QS-21 species having a monoisotopic molecular weight (m/z) of 1856 (“QS-21 1856”) with negative mode electrospray mass spectrometry (which may exist as A and B isomers):

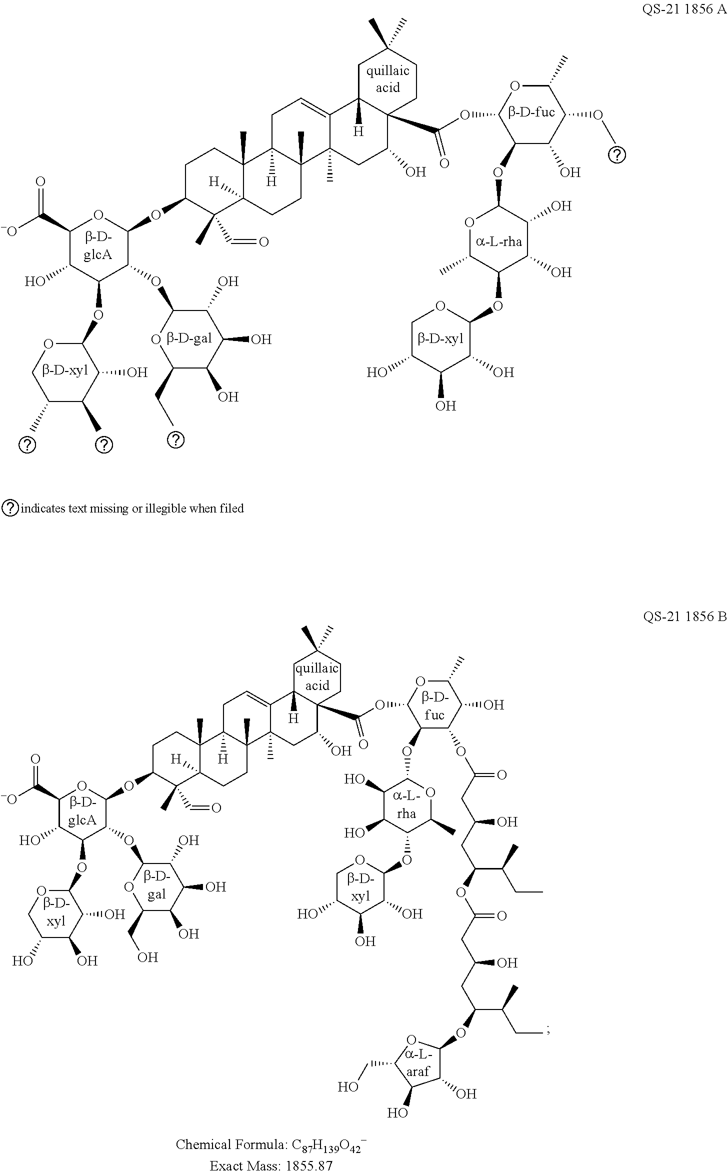

(iii) the rhamnose chemotype QS-21 species having a monoisotopic molecular weight (m/z) of 2002 (“QS-21 2002”) with negative mode electrospray mass spectrometry (which may exist as A and B isomers, and V1 apiose and V2 xylose isomers):

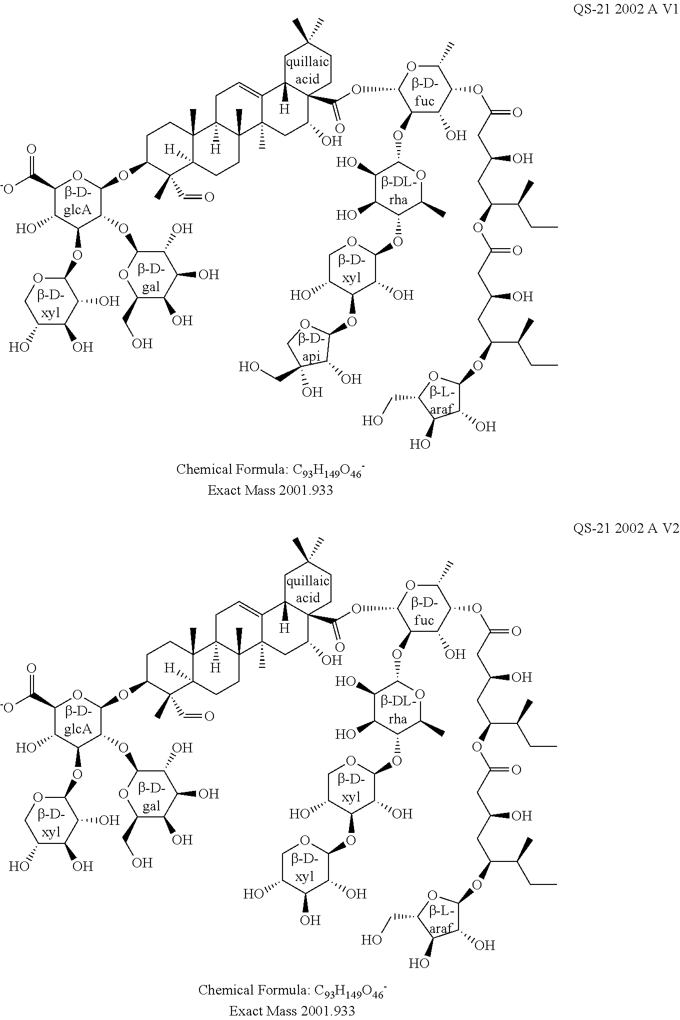

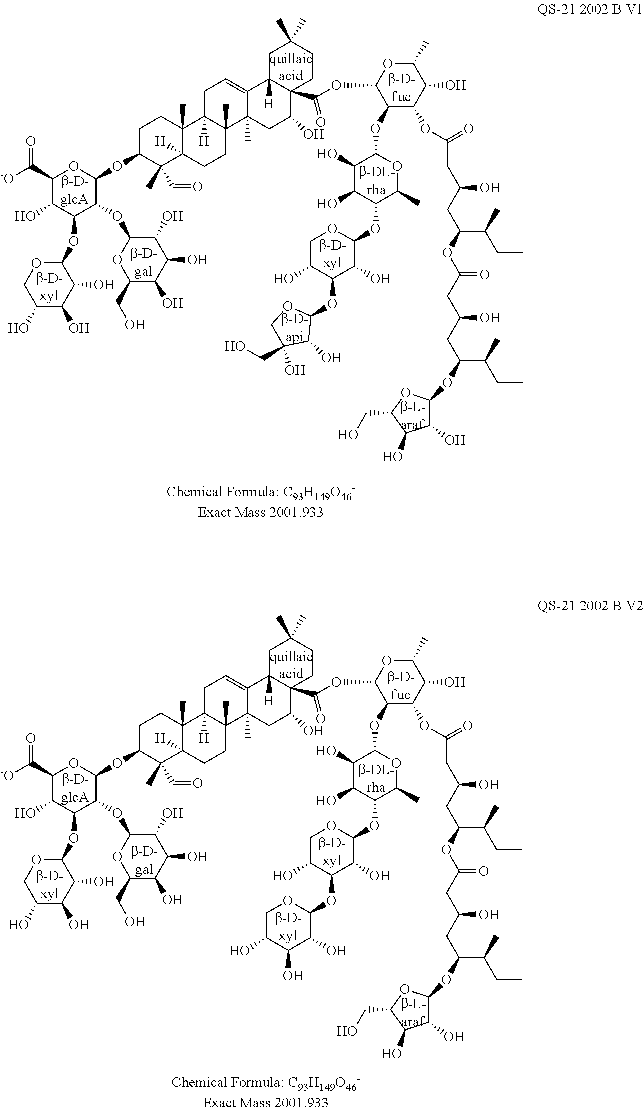

In some embodiments, the saponins produced by the method of the invention are one or more of the above QS-21 saponin species from the QS-21 saponin family. In specific embodiments, the saponins produced by the methods of the invention are one or more from QS-21 1988 A V1, QS-21 1988 A V2, QS-21 1988 B V1 and QS-21 1988 B V2.

In further embodiments, the invention provides a suspension cell line of plant cells capable of producing one or more of the above QS-21 saponin species from the QS-21 saponin family. In specific embodiments, the saponins produced by the suspension cell line of plant cells of the invention are one or more from QS-21 1988 A V1, QS-21 1988 A V2, QS-21 1988 B V1 and QS-21 1988 B V2.

The invention is further illustrated by reference to the following clauses:
Clause 1. A method for converting non-producing plant cells capable of naturally synthesizing saponins containing a quillaic acid triterpenoid aglycone into plant cells producing saponins, said method comprising at least the following steps:
   i) culturing the non-producing plant cells in a culture medium comprising a source of nitrogen,
   ii) depleting the culture medium from any nitrogen source, and
   iii) eliciting the production of saponins with at least one elicitor.
Clause 2. A method for producing saponins containing a quillaic acid triterpenoid aglycone, said method comprising at least the following steps:
   i) culturing plant cells capable of naturally synthesizing saponins containing a quillaic acid triterpenoid aglycone in a culture medium comprising a source of nitrogen,
   ii) depleting the culture medium from any nitrogen source,
   iii) eliciting the production of saponins with at least one elicitor, and
   iv) recovering the saponins produced.
Clause 3. The method according to clause 1 or clauses 2, wherein the plant cells are grown in suspension.
Clause 4. The method according to clauses 1 to 3, wherein the plant cells are suspension cell lines.
Clause 5. The method according to clauses 1 to 4, wherein the source of nitrogen in the culture medium in step i) comprises at least nitrates.
Clause 6. The method according to clause 5, wherein the nitrates are KNO₃ and/or NH₄NO₃.
Clause 7. The method according to clause 6, wherein the nitrates are KNO₃.
Clause 8. The method according to clauses 1 to 6, wherein the source of nitrogen comprises KNO₃ and NH₄NO₃.
Clause 9. The method according to clauses 5 to 8, wherein the source of nitrogen further comprises NH₄Cl.
Clause 10. The method according to clauses 1 to 8, wherein the source of nitrogen does not comprise NH₄Cl.
Clause 11. The method according to clauses 1 to 10, wherein the total concentration of the source of nitrogen is from 10 mM to 50 mM.
Clause 12. The method according to clause 11, wherein the total concentration of the source of nitrogen is from 15 mM to 40 mM.
Clause 13. The method according to clause 12, wherein the total concentration of the source of nitrogen is from 20 mM to 30 mM.
Clause 14. The method according to clauses 11 to 13, wherein the total concentration of the source of nitrogen is about 25 mM.
Clause 15. The method according to clauses 11 and 12, wherein the total concentration of the source of nitrogen is about 40 mM.
Clause 16. The method according to clauses 5 to 15, wherein KNO₃ concentration, when present, is from 5 mM to 30 mM.
Clause 17. The method according to clause 16, wherein KNO₃ concentration is from 10 mM to 20 mM.
Clause 18. The method according to clause 17, wherein KNO₃ concentration is about 15 mM.
Clause 19. The method according to clause 17, wherein KNO₃ concentration is about 20 mM.
Clause 20. The method according to clauses 5 to 19, wherein NH₄NO₃ concentration, when present, is from 5 mM to 30 mM.
Clause 21. The method according to clause 20, wherein NH₄NO₃ concentration is from 10 mM to 20 mM.
Clause 22. The method according to clause 21, wherein NH₄NO₃ concentration is about 10 mM.
Clause 23. The method according to clause 20, wherein NH₄NO₃ concentration is about 20 mM.
Clause 24. The method according to clauses 5 to 23, wherein NH₄Cl concentration, when present, is from 5 to 20 mM.
Clause 25. The method according to clause 24, wherein NH₄Cl concentration is about 10 mM.
Clause 26. The method according to clauses 1 to 8, wherein the nitrogen source comprises 15 mM of KNO₃ and 10 mM of NH₄NO₃.
Clause 27. The method according to clauses 1 to 26, wherein the culture medium in step i) comprises one or more of sucrose, glucose and fructose as carbon source.
Clause 28. The method according to clause 27, wherein the carbon source is sucrose, glucose and fructose.
Clause 29. The method according to clause 27, wherein the carbon source does not comprise sucrose.
Clause 30. The method according to clauses 27 to 29, wherein the total concentration of the carbon source ranges from 40 mM to 100 mM.
Clause 31. The method according to clause 30, wherein the total concentration of the carbon source ranges 50 mM to 90 mM.
Clause 32. The method according to clause 31, wherein the total concentration of the carbon source ranges from 60 mM to 80 mM.
Clause 33. The method according to clause 31, wherein the total concentration of the carbon source is about 60 mM.
Clause 34. The method according to clause 31, wherein the total concentration of the carbon source is about 70 mM.
Clause 35. The method according to clauses 27 to 34, wherein the concentration of sucrose, when present, ranges from 5 mM to 100 mM.
Clause 36. The method according to clause 35, wherein the concentration of sucrose ranges from 5 mM to 20 mM.
Clause 37. The method according to clause 35, wherein the concentration of sucrose ranges from 10 mM to 80 mM.
Clause 38. The method according to clause 37, wherein the concentration of sucrose ranges from 20 mM to 60 mM.
Clause 39. The method according to clause 36, wherein the concentration of sucrose is about 10 mM.
Clause 40. The method according to clauses 27 to 39, wherein the concentration of glucose, when present, ranges from 5 mM to 60 mM.
Clause 41. The method according to clause 40, wherein the concentration of glucose ranges from 15 mM to 60 mM.
Clause 42. The method according to clause 40, wherein the concentration of glucose ranges from 20 mM to 40 mM.
Clause 43. The method according to clauses 28 to 39, wherein the concentration of glucose, when present, ranges from 10 mM to 80 mM.
Clause 44. The method according to clauses 40 to 43, wherein the concentration of glucose is about 30 mM.
Clause 45. The method according to clause 43, wherein the concentration of glucose is about 60 mM.
Clause 46. The method according to clauses 27 to 45, wherein the concentration of fructose, when present, ranges from 5 mM to 60 mM.
Clause 47. The method according to clause 46, wherein the concentration of fructose ranges from 15 mM to 60 mM.
Clause 48. The method according to clause 47, wherein the concentration of fructose ranges from 20 mM to 40 mM.
Clause 49. The method according to clauses 27 to 45, wherein the concentration of fructose, when present, ranges from 10 mM to 80 mM.
Clause 50. The method according to clauses 46 to 49, wherein the concentration of fructose is about 30 mM.
Clause 51. The method according to any one of clauses 1 to 50, wherein the culture medium in step i) comprises hormones.
Clause 52. The method according to clause 51, wherein the hormones are one or more auxins and/or one or more cytokinins.
Clause 53. The method according to clause 52, wherein the one or more auxins is NAA or 2,4-D.
Clause 54. The method according to clause 52, wherein the one or more cytokinins is BA.
Clause 55. The method according to clauses 1 to 54, wherein the culture medium in step i) comprises at least 2,4-D.
Clause 56. The method according to clauses 1 to 54, wherein the culture medium in step i) comprises at least 2,4-D and NAA.
Clause 57. The method according to clauses 1 to 56, wherein the culture medium in step i) comprises 2,4-D, NAA and BA.
Clause 58. The method according to clauses 53 to 57, wherein the concentration of NAA, 2,4-D and BA, when present, ranges from 0.2 mg/L to 0.8 mg/L.
Clause 59. The method according to clause 58, wherein the concentration of NAA, 2,4-D and BA is about 0.5 mg/L.
Clause 60. The method according to clauses 1 to 59, wherein the culture medium in step i) comprises further macronutrients selected from CaCl₂, MgSO₄ and KH₂PO₄.
Clause 61. The method according to clauses 1 to 60, wherein the culture medium in step i) comprises at least KH₂PO₄.
Clause 62. The method according to clause 61, wherein the concentration of KH₂PO₄ ranges from 0.6 mM to 5 mM.
Clause 63. The method according to clause 62, wherein the concentration of KH₂PO₄ ranges from 1.5 mM to 5 mM.
Clause 64. The method according to clause 62, wherein the concentration of KH₂PO₄ is about 1.25 mM.
Clause 65. The method according to clause 63, wherein the concentration of KH₂PO₄ is about 2.5 mM.
Clause 66. The method according to clause 63, wherein the concentration of KH₂PO₄ is 5 mM.
Clause 67. The method according to clauses 1 to 66, wherein the culture medium in step i) comprises micronutrients selected from KI, H₃BO₃, MnSO₄, ZnSO₄, Na₂MoO₄, CuSO₄, CoCl₂, DeSO₄, or Na₂EDTA.
Clause 68. The method according to clauses 1 to 67, wherein the culture medium in step i) comprises vitamins selected from myo-inositol, nicotinic acid, pyrodixine-HCl, or thiamine-HCl.
Clause 69. The method according to clauses 1 to 68, wherein the culture medium in step i) comprises CaCl₂, KH₂PO₄, KNO₃, MgSO₄, NH₄NO₃, sucrose, glucose, fructose, NAA and 2,4-D.
Clause 70. The method according to clauses 1 to 69, wherein the culture medium in step i) is Medium 4.
Clause 71. The method according to clauses 1 to 68, wherein the culture medium in step i) comprises CaCl₂, KH₂PO₄, KNO₃, MgSO₄, NH₄NO₃, sucrose, NAA and 2,4-D.
Clause 72. The method according to clause 71, wherein the culture medium is Medium 6.
Clause 73. The method according to clauses 1 to 72, wherein the osmolality of the culture medium in step i) and/or step ii) and/or step iii) ranges between 100 and 220 mOsm.
Clause 74. The method according to clauses 1 to 73, wherein the osmolality is between 180 and 200 mM.
Clause 75. The method according to clauses 1 to 74, wherein the plant cells in step i) are cultured until reaching a PCV ranging from 10% to 70%.
Clause 76. The method according to clause 75, wherein the PCV ranges from 15% to 30%.
Clause 77. The method according to clause 75, wherein the PCV ranges from 20% and 60%.
Clause 78. The method according to clause 77, wherein the PCV ranges from 30% to 50%.
Clause 79. The method according to clause 75, wherein the PCV is about 15%.
Clause 80. The method according to clause 75, wherein the PCV is about 20%.
Clause 81. The method according to clause 77, wherein the PCV is about 30%.
Clause 82. The method according to clause 77, wherein the PCV is about 40%.
Clause 83. The method according to clauses 1 to 82, wherein step i) is from 4 to 8 days, or longer.
Clause 84. The method according to clause 83, wherein step i) is from 5 to 7 days.
Clause 85. The method according to clause 83, wherein step i) is from 4 to 5 days.
Clause 86. The method according to clauses 1 to 85, wherein step ii) is performed by replacing the culture medium at the end of step i) with a culture medium containing no source of nitrogen and maintaining the cells in the replacing culture medium.
Clause 87. The method according to clauses 1 to 86, wherein step ii) is performed by replacing the culture medium at the end of step i) with a culture medium containing 5 mM or less of nitrogen source and maintaining the cells in the replacing culture medium.
Clause 88. The method according to clause 87, wherein the replacing culture medium contains between 0.5 mM to 5 mM of nitrogen source.
Clause 89. The method according to clause 88, wherein the replacing culture medium contains between 1.25 mM to 2.5 mM of nitrogen source.
Clause 90. The method according to clauses 88, wherein the replacing culture medium contains about 1.25 mM of nitrogen source.
Clause 91. The method according to clause 88, wherein the replacing culture medium contains about 2.5 mM of nitrogen source.
Clause 92. The method according to clause 88, wherein the replacing culture medium contains about 5 mM of nitrogen source.
Clause 93. The method according to clause 87 to 92, wherein the nitrogen source in the replacing medium is one or more of KNO₃, NH₄NO₃ or NH₄Cl.
Clause 94. The method according to clauses 87 to 93, wherein the nitrogen source in the replacing medium is KNO₃.
Clause 95. The method according to clauses 87 to 94, wherein the nitrogen source in the replacing medium is NH₄NO₃.
Clause 96. The method according to clauses 87 to 95, wherein the nitrogen source in the replacing medium is NH₄Cl.
Clause 97. The method according to clauses 86 to 96, wherein the replacing culture medium comprises KCl.
Clause 98. The method according to clause 97, wherein KCl concentration is from 10 to 20 mM.
Clause 99. The method according to clauses 86 to 98, wherein the replacing culture medium comprises one or more nutrients as in clauses 27 to 50 and 60-67, one or more hormones as in clauses 51 to 59, and one or more vitamins as in clause 68.
Clause 100. The method according to clauses 86 to 99, wherein the replacing culture medium is Medium 1.
Clause 101. The method according to clauses 1 to 100, wherein step ii) is from 1 to 9 days.
Clause 102. The method according to clause 101, wherein step ii) is from 2 to 7 days.
Clause 103. The method according to clause 101, wherein step ii) is from 5 to 7 days.
Clause 104. The method according to clause 103, wherein step ii) is from 3 to 6 days.
Clause 105. The method according to clauses 104 wherein step ii) is 4 days Clause 106. The method according to clause 104 wherein step ii) is 5 days.
Clause 107. The method according to clauses 104, wherein step ii) is 7 days.
Clause 108. The method according to clauses 86 to 100, wherein the cells are maintained in the replacing culture medium for 1 to 9 days
Clause 109. The method according to clause 105, wherein the cells are maintained in the replacing culture medium for 2 to 7 days.
Clause 110. The method according to clause 109, wherein the cells are maintained in the replacing culture medium for 5 to 7 days.
Clause 111. The method according to clause 110, wherein the cells are maintained in the replacing culture medium for 3 to 6 days.
Clause 112. The method according to clause 111 wherein the cells are maintained in the replacing culture medium for 4 days.
Clause 113. The method according to clause 111 wherein the cells are maintained in the replacing culture medium for 5 days.
Clause 114. The method according to clause 109, wherein the cells are maintained in the replacing culture medium for 7 days.
Clause 115. The method according to clauses 1 to 85, wherein step ii) is performed by letting the cells naturally consume the source of nitrogen included in the culture medium used in step i) down to a residual level, with no further replenishment of the culture medium with any nitrogen source, and maintaining the cells in the consumed culture medium.
Clause 116. The method according to clause 115, wherein the residual level of the nitrogen source is 2-fold, 4-fold, or 8-fold less, as compared with the concentration of the nitrogen source included in the culture medium in step i).
Clause 117. The method according to clause 115, wherein the residual level of the nitrogen source is less than 5 mM.
Clause 118. The method according to clause 117, wherein the residual level of nitrogen source is less than 2.5 mM.
Clause 119. The method according to clause 118, wherein the residual level of nitrogen source is less than 1 mM.
Clause 120. The method according to clause 117, wherein the residual level of nitrogen source is between 1 and 2 mM.
Clause 121. The method according to clauses 115 to 119, wherein the residual level of nitrogen source is undetectable.
Clause 122. The method according to clauses 115 to 121, wherein the nitrogen source in the consumed culture medium is one or more of KNO₃, NH₄NO₃ or NH₄Cl.
Clause 123. The method according to clause 115 to 122, wherein the nitrogen source in the consumed medium is KNO₃.
Clause 124. The method according to clauses 115 to 123, wherein the nitrogen source in the consumed medium is NH₄NO₃.
Clause 125. The method according to clauses 115 to 123, wherein the nitrogen source is KNO₃ and NH₄NO₃.
Clause 126. The method according to clauses 115 to 125, wherein the nitrogen source in the consumed medium is NH₄Cl.
Clause 127. The method according to clauses 1 to 85 and 115 to 126, wherein step ii) is from 5 to 20 days.
Clause 128. The method according to clause 127, wherein step ii) is from 6 to 19 days.
Clause 129. The method according to clause 128, wherein step ii) is from 7 to 18 days.
Clause 130. The method according to clause 129, wherein step ii) is from 8 to 16 days.
Clause 131. The method according to clause 130, wherein step ii) is from 9 to 15 days.
Clause 132. The method according to clause 131, wherein step ii) is 10 days.
Clause 133. The method according to clause 131, wherein step ii) is 11 days.
Clause 134. The method according to clause 131, wherein step ii) is 12 days.
Clause 135. The method according to clause 131, wherein step ii) is 13 days.
Clause 136. The method according to clause 131, wherein step ii) is 14 days.
Clause 137. The method according to clauses 115 to 136, wherein the cells are maintained in the consumed culture medium from 2 to 7 days.
Clause 138. The method according to clause 137, wherein the cells are maintained in the consumed culture medium from 5 to 7 days
Clause 139. The method according to clause 138 wherein the cells are maintained in the consumed culture medium from 3 to 6 days.
Clause 140. The method according to clause 139, wherein the cells are maintained in the consumed culture medium for 4 days.
Clause 141. The method according to clause 139, wherein the cells are maintained in the consumed culture medium for 5 days.
Clause 142. The method according to clause 138, wherein the cells are maintained in the consumed culture medium for 7 days.
Clause 143. The method according to clauses 1 to 142, wherein the culture medium in step ii) is monitored and replenished with glucose to maintain a minimum level of about 15 mM.
Clause 144. The method according to clauses 1 to 143, wherein the culture medium in step ii) is monitored and replenished with KH₂PO₄ to maintain a minimum level of about 2.5 mM.
Clause 145. The method according to clauses 1 to 144, wherein the at least one elicitor in step iii) is a moncocarboxylic compound-type elicitor.
Clause 146. The method according to clause 145, wherein the at least one elicitor is one or more of 5-chlorosalicyclic acid, salicyclic acid, acetylsalicyclic acid, and a methyl ester.
Clause 147. The method according to clauses 145 and 146, wherein the at least one elicitor is methyl jasmonate (MeJa).
Clause 148. The method according to clauses 1 to 147, wherein the concentration of the at least one elicitor in step iii) ranges from 0.5 to 12 μM.
Clause 149. The method according to clause 148, wherein the concentration of the at least one elicitor in step iii) is from 1 to 8 μM.
Clause 150. The method according to clause 149, wherein the concentration of the at least one elicitor in step iii) is from 2 to 6 μM.
Clause 151. The method according to clause 150, wherein the concentration of the at least one elicitor in step iii) is from 3 to 5 μM.
Clause 152. The method according to clause 149, wherein the concentration of the at least one elicitor in step iii) is from 1 to 3 μM.
Clause 153. The method according to clause 152, wherein the concentration of the at least one elicitor in step iii) is about 2 μM.
Clause 154. The method according to clause 152, wherein the concentration of the at least one elicitor in step iii) is about 3 μM.
Clause 155. The method according to clause 150, wherein the concentration of the at least one elicitor in step iii) is about 6 μM.
Clause 156. The method according to clauses 1 to 147, wherein the concentration of the at least one elicitor in step iii) ranges from 0.5 to 12 μM/PCV %.
Clause 157. The method according to clause 156, wherein the concentration of the at least one elicitor in step iii) is from 1 to 8 μM/PCV %.
Clause 158. The method according to clause 157, wherein the concentration of the at least one elicitor in step iii) is from 2 to 6 μM/PCV %.
Clause 159. The method according to clause 158, wherein the concentration of the at least one elicitor in step iii) is from 3 to 5 μM/PCV %.
Clause 160. The method according to clause 157, wherein the concentration of the at least one elicitor in step iii) is from 1 to 3 μM/PCV %.
Clause 161. The method according to clause 160, wherein the concentration of the at least one elicitor in step iii) is 2 μM/PCV %.
Clause 162. The method according to clause 160, wherein the concentration of the at least one elicitor in step iii) is 3 μM/PCV %.
Clause 163. method according to clause 158, wherein the concentration of the at least one elicitor in step iii) is 6 μM/PCV %.
Clause 164. The method according to clauses 1 to 163, wherein the at least one elicitor in step iii) is added directly to the cells at the end of step ii).
Clause 165. The method according to clauses 1 to 164, wherein step iii) is between 1 to 14 days.
Clause 166. The method according to clause 165, wherein step iii) is between 2 to 10 days.
Clause 167. The method according to clause 166, wherein step iii) is between 3 to 8 days.
Clause 168. The method according to clause 167, wherein step iii) is between 4 to 6 days.
Clause 169. The method according to clause 168, wherein step iii) is 5 days.
Clause 170. The method according to clause 167, wherein step iii) is 7 days.
Clause 171. The method according to clause 166, wherein step iii) is 10 days.
Clause 172. The method according to clauses 1 to 164, wherein the plant cells are harvested between 1 to 14 days after addition of the at least one elicitor.
Clause 173. The method according to clause 172, wherein the plant cells are harvested between 2 to 10 days after the addition of the at least one elicitor.
Clause 174. The method according to clause 173, wherein the plant cells are harvested between 3 to 8 days after addition of the at least one elicitor.
Clause 175. The method according to clause 174, wherein the plant cells are harvested between 4 to 6 days after addition of the at least one elicitor.
Clause 176. The method according to clause 173, wherein the plant cells are harvested 5 days after addition of the at least one elicitor.
Clause 177. The method according to clause 173, wherein the plant cells are harvested 7 days after addition of the at least one elicitor.
Clause 178. The method according to clause 173, wherein the plant cells are harvested 10 days after addition of the at least one elicitor
Clause 179. The method according to clauses 164 to 178, wherein the at least one elicitor is further added every other day.
Clause 180. The method according to clauses 1 to 179, wherein the culture medium in step iii) is monitored and replenished with glucose to maintain a minimum level of about 15 mM.
Clause 181. The method according to clauses 1 to 180, wherein the culture medium in step iii) is monitored and replenished with KH₂PO₄ to maintain a minimum level of about 2.5 mM.
Clause 182. The method according to clauses 1 to 181, wherein the plant cells in step i) and/or step ii) and/or step iii) are cultured at a temperature ranging from 20° C. to 30° C.
Clause 183. The method according to clauses 1 to 182, wherein the plant cells in step i), step ii) and step iii) are cultured at a temperature of about 25° C.
Clause 184. The method according to clauses 1 to 183, wherein the plant cells in step i) and/or step ii) and/or step iii) are agitated at a speed ranging from 40 to 60 rpm.
Clause 185. The method according to clause 184, wherein the cells in step i), step ii) and step iii) are agitated at a speed of about 50 rpm.
Clause 186. The method according to clauses 1 to 185, wherein the plant cells are cultured in shake flasks.
Clause 187. The method according to clauses 1 to 185, wherein the plant cells are cultured in a bioreactor.
Clause 188. Plant cells obtainable by the method according to clauses 1 and 3 to 187.
Clause 189. A suspension of plant cells obtainable by the method according to clauses 1 and 3 to 187.
Clause 190. A suspension cell line obtainable by the method according to clauses 1 and 3 to 187.
Clause 191. A suspension of plant cells naturally synthesizing quillaic acid-based triterpenoid saponins capable of producing such saponins at a volumetric productivity of at least 5 mg/L.
Clause 192. The suspension of plant cells according to clause 191, wherein the volumetric productivity is at least 10 mg/L.
Clause 193. The suspension of plant cells according to clause 192, wherein the volumetric productivity is at least 20 mg/L.
Clause 194. The suspension of plant cells according to clause 193, wherein the volumetric productivity is at least 40 mg/L.
Clause 195. The suspension of plant cells according to clause 194, wherein the volumetric productivity is at least 50 mg/L.
Clause 196. The suspension of plant cells according to clauses 191 to 195, wherein the suspension produces quillaic acid-based triterpenoid saponins at such volumetric productivity.
Clause 197. A suspension cell line of plant cells naturally synthesizing quillaic acid-based triterpenoid saponins capable of producing such saponins at a volumetric productivity of at least 10 mg/L.
Clause 198. The suspension cell line of plant cells according to clause 197, wherein the volumetric productivity is at least 20 mg/L.
Clause 199. The suspension cell line of plant cells according to clause 198, wherein the volumetric productivity is at least 40 mg/L.
Clause 200. The suspension cell line of plant cells according to clause 199, wherein the volumetric productivity is at least 50 mg/L.
Clause 201. The suspension cell line of plant cells according to clauses 197 to 200, wherein the suspension cell line produces quillaic acid-based triterpenoid saponins at such volumetric productivity.
Clause 202. The method according to clauses 1 to 187, the plant cells, the suspension of plant cells, or the suspension cell line according to clauses 189 to 201, wherein the plant cells are from the genus Quillaja.
Clause 203. The method, plant cells, suspension of plant cells, or suspension cell line according to clause 202, wherein the plant cells are from the species Quillaja saponaria.
Clause 204. The method, plant cells, suspension of plant cells or suspension cell line according to clause 202 wherein the plant cells are from the species Quillaja brasiliensis.
Clause 205. The method, plant cells, suspension of plant cells, or suspension cell line according to clauses 202 to 204, wherein the plant cells are cambial meristematic cells (CMC).
Clause 206. The method, plant cells, suspension of plant cells, or suspension cell line according to clauses 202 to 206, wherein the saponins are one or more saponin species from the QS-7 saponin family, the QS-17 saponin family, the QS-18 saponin family and/or the QS-21 saponin family.
Clause 207. The method, plant cells, suspension of plant cells, or suspension cell line according to clause 206, wherein the saponins are one or more saponin species from the QS-7 saponin family.
Clause 208. The method, plant cells, suspension of plant cells, or suspension cell line according to clause 207 wherein the saponins are one or more saponin species from QS-7 1862 V1, QS-7 1862 V2, Xyl-QS-7 1730, QS-7 1700, Xyl-QS-7 1568, QS-7 1554, QS-7 1716, QS-7 1876 V1, QS-7 1876 V2, QS-7 1714 V1, Rha-QS-7 1568 V1, Rha-QS-7 1730.
Clause 209. The method, plant cells, suspension of plant cells, or suspension cell line according to clause 207 wherein the saponins are one or more saponin species from QS-7 1862 V1, QS-7 1862 V2, Xyl-QS-7 1730, QS-7 1700, Xyl-QS-7 1568, QS-7 1554, QS-7 1716, QS-7 1876 V1, QS-7 1876 V2, QS-7 1714 V1, QS-7 1714 V2, Rha-QS-7 1568 V1, Rha-QS-7 1568 V2, Rha-QS-7 1730, QS-7 1582.
Clause 210. The method, plant cells, suspension of plant cells, or suspension cell line according to clause 206, wherein the saponins are one or more saponin species from the QS-17 saponin family.
Clause 211. The method, plant cells, suspension of plant cells, or suspension cell line according to clause 210, wherein the saponins are one or more saponin species from QS-17 2296 A V1, QS-17 2296 A V2, QS-17 2296 B V1, QS-17 2296 B V2, QS-17 2164 A, QS-17 2164 B, QS-17 2310 A V1, QS-17 2310 A V2, QS-17 2310 B V1, QS-17 2310 B V2.
Clause 212. The method, plant cells, suspension of plant cells, or suspension cell line according to clause 210 wherein the saponins are one or more saponin species from QS-17 2296 A V1, QS-17 2296 A V2, QS-17 2296 B V1, QS-17 2296 B V2, QS-17 2164 A, QS-17 2164 B, QS-17 2310 A V1, QS-17 2310 A V2, QS-17 2310 B V1, QS-17 2310 B V2, QS-7 2134 A V1, QS-7 2134 A V2, QS-7 2134 B V1, QS-7 2134 B V2, QS-7 2148 A V1, QS-7 2148 A V2, QS-7 2148 B V1, QS-7 2148 B V2.
Clause 213. The method, plant cells, suspension of plant cells, or suspension cell line according to clause 206, wherein the saponins are one or more saponin species from the QS-18 saponin family.
Clause 214. The method, plant cells, suspension of plant cells, or suspension cell line according to clause 213, wherein the saponins are one or more from QS-18 2150 A V1, QS-18 2150 A V2, QS-18 2150 B V1, QS-18 2150 B V2, QS-18 2018 A, QS-18 2018 B, QS-18 2164 A V1, QS-18 2164 A V2, QS-18 2164 B V1, QS-18 2164 B V2.
Clause 215. The method, plant cells, suspension of plant cells, or suspension cell line according to clause 213 wherein the saponins are one or more from QS-18 2150 A V1, QS-18 2150 A V2, QS-18 2150 B V1, QS-18 2150 B V2, QS-18 2018 A, QS-18 2018 B, QS-18 2164 A V1, QS-18 2164 A V2, QS-18 2164 B V1, QS-18 2164 B V2, QS-18 2032 A, QS-18 2032 B.
Clause 216. The method, plant cells, suspension of plant cells, or suspension cell line according to clauses 214 and 215, wherein the saponins are one or more from QS-18 2150 A V1, QS-18 2150 A V2, QS-18 2150 B V1 and QS-18 2150 B V2.
Clause 217. The method, plant cells, suspension of plant cells, or suspension cell line according to clause 206, wherein the saponins are one or more saponin species from the QS-21 saponin family.
Clause 218. The method, plant cells, suspension of plant cells, or suspension cell line according to clause 217, wherein the saponins are one or more from QS-21 1988 A V1, QS-21 1988 A V2, QS-21 1988 B V1 and QS-21 1988 B V2, QS-21 1856 A, QS-21 1856 B, QS-21 2002 A V1, QS-212002 B V1, QS-21 2002 A V2, QS-21 2002 B V2.
Clause 219. The method, plant cells, suspension of plant cells, or suspension cell line according to 218, wherein the saponins are one or more from QS-21 1988 A V1, QS-21 1988 A V2, QS-21 1988 B V1 and QS-21 1988 B V2.
Clause 220. A method for preparing an adjuvant comprising saponins, comprising the following steps:
   a) producing saponins according to the method as claimed in clauses 2 to 187 and clauses 202 to 219, and
   b) formulating the recovered saponin as an adjuvant.
Clause 221. The method according to clause 220, wherein the saponin adjuvant formulation is a liposomal formulation.
Clause 222. The method according to clauses 220 and 221, wherein the saponins are one or more of the QS-21 saponin family.

### Terms

Unless otherwise explained in the context of this disclosure, all technical and scientific terms used herein have the same meaning as commonly understood by one of ordinary skill in the art to which this disclosure belongs.

The singular terms “a,” “an,” and “the” include plural referents unless context clearly indicates otherwise. Similarly, the word “or” is intended to include “and” unless the context clearly indicates otherwise. The term “plurality” refers to two or more. It is further to be understood that all base sizes or amino acid sizes, and all molecular weight or molecular mass values, given for nucleic acids or polypeptides are approximate, and are provided for description.

In respect of numerical values, the terms “approximately”, “around” or “about” will typically mean a value within plus or minus 10 percent of the stated value, especially within plus or minus 5 percent of the stated value and in particular the stated value.

Additionally, numerical limitations given with respect to concentrations or levels of a substance, such as an antigen, are intended to be approximate. Thus, where a concentration is indicated to be at least (for example) 200 pg, it is intended that the concentration be understood to be at least approximately (or “about” or “˜”) 200 pg. The term “comprises” means “includes.” Thus, unless the context requires otherwise, the word “comprises,” and variations such as “comprise” and “comprising” will be understood to imply the inclusion of a stated compound or composition (e.g., nucleic acid, polypeptide, antigen) or step, or group of compounds or steps, but not to the exclusion of any other compounds, composition, steps, or groups thereof.

The present invention will now be further described by means of the following non-limiting examples.

### EXAMPLES

### Example 1—Production of Calli

Calli were established from the cambial meristematic cells of selected Quillaja Saponaria plants.

A young shoot from a growing plant was cut into small pieces. The outer layer was stripped away by surface sterilization in order to expose the cambial layer. Said cambial layer was then laid on agar plates containing Murashige and Skoog (MS) medium supplemented with the plant hormones 1-Naphthaleneacetic acid (NAA) and 6- Benzylaminopurine (BA) at 0.5 mg/L each.

The plates were then incubated at 25° C. in the dark for 4 weeks, after which time it was sub-cultured to fresh solid MS medium (plus the above plant hormones) and incubated at 25° C. in the dark. After 8 weeks, the growing cambial cells were separated from the hard callus and transferred to fresh MS medium (plus the above plant hormones). The resulting callus was continually sub-cultured as above, every 4 weeks, to maintain viability.

### Example 2—Initiation and Maintenance of Suspension Plant Cells

Suspension plant cell cultures, or suspension cell lines, were initiated by inoculating liquid medium with callus material in shake flasks. A 10% inoculum was used, e.g. 3 g of callus material was inoculated into 30 ml liquid MS medium containing NAA and 2,4-dichlorophenoxyacetic acid (2,4-D), at 0.5 mg/L each. Liquid volume as a percentage of the total flask volume was fixed at no more than 20%. The liquid suspension flasks were incubated, at a temperature of 25° C. on a shaker set at 200 rpm, for 14 days before being sub-cultured again into liquid medium. Subculture was achieved by allowing the large aggregates in the cell suspension to settle, before drawing off the liquid containing the fine cells in suspension into a centrifuge tube. This suspension was centrifuged, the supernatant was poured off, and the remaining cell pellet (packed cell volume—PCV) was re-suspended in fresh MS medium (plus NAA and 2,4-D) (see Medium 6 in Table 1 below). The volume of fresh medium added was such that the final cell concentration is 10% (as PCV) of the final volume. Sub-cultured suspension flasks were incubated at a temperature of 25° C. on a shaker set at 200 rpm for 14 days before being sub-cultured once again, as described above. Suspension plant cell cultures are maintained by further sub-culturing every 9 to 14 days. Sub-culturing may also be referred to as “passages” (P), with P0 corresponding to the moment where a given cell line transitioned from the callus stage to the ability to grow in suspension in a liquid medium.

### Example 3—Effect of Nitrogen Depletion and Elicitation

The following diagram provides a schematic view illustrative of embodiments of the method of the invention as carried out in the Experiments 1 to 9 below.

### 3.1 Experiment 1

5 different cultures of the same suspension plant cell line named “CMC40B6” (established as described in Example 1 and Example 2) were grown in parallel in a maintenance culture medium, such as Medium 4 below, until the PCV of the cultures reached 20%. At this point in time (Day 0), the culture medium of all cultures was removed and replaced with either Medium 1, Medium 2, Medium 3, Medium 4 or Medium 5 containing varying levels of nitrogen source and variable sources of nitrogen (as described in Table 1 below), and let grow for 5 more days. At Day 5, the PCV % of each culture was measured and 3 μM/PCV % of MeJa was added directly to the culture medium of each of the 5 cultures, and let grow for 4 more days. Growth of the cultures was analyzed by sampling the cultures at Day 5, Day 7 and Day 8 and measuring the PCV. At Day 9 (i.e. 4 days post-elicitation), cells were harvested and disrupted (as described in Example 4), and the QS-18 saponin content was analysed and measured (as described in Example 5).

TABLE 1
Composition of culture media
DEPLETION CONDITIONS

| Medium Composition | Medium 1 | Medium 2 | Medium 3 | Medium 4 | Medium 5 | Medium 6 |
|---|---|---|---|---|---|---|
| Micronutrients* | 1-fold | 1-fold | 1-fold | 1-fold | 1-fold | 1-fold |
| Vitamins** | 1-fold | 1-fold | 1-fold | 1-fold | 1-fold | 1-fold |
| CaCl₂ (mM) | 2.99 | 2.99 | 2.99 | 2.99 | 2.99 | 2.99 |
| KH₂PO₄ (mM) | 2.5 | 2.5 | 2.5 | 2.5 | 2.5 | 1.25 |
| KNO₃ (mM) | 0 | 25 | 0 | 15 | 25 | 18.8 |
| KCl (mM) | 15 | 15 | 15 | 0 | 15 | 0 |
| MgSO₄ (mM) | 1.5 | 1.5 | 1.5 | 1.5 | 1.5 | 1.5 |
| NH₄NO₃ (mM) | 0 | 0 | 0 | 10 | 0 | 20.51 |
| NH₄Cl (mM) | 0 | 0 | 15 | 0 | 10 | 0 |
| Glucose (mM) | 32 | 32 | 32 | 32 | 32 | 0 |
| Fructose (mM) | 32 | 32 | 32 | 32 | 32 | 0 |
| Sucrose (mM) | 10 | 10 | 10 | 10 | 10 | 87.64 |
| NAA*** (mg/L) | 0.5 | 0.5 | 0.5 | 0.5 | 0.5 | 0.5 |
| 2,4-D**** (mg/L) | 0.5 | 0.5 | 0.5 | 0.5 | 0.5 | 0.5 |

*“Micronutrients” is a MS Basal Salt Micronutrient Solution 10 × purchased from Sigma Aldrich (ref. M0529), and diluted 10 times (i.e. “1-fold”)
**“Vitamins” is a MS vitamin powder 1000 × purchased from Sigma Aldrich (ref. M7150), and diluted 1000 times (i.e. “1-fold”)
***“NAA” is 1-Napthaleneacetic acid
****“2,4-D” is 2,4-dichlorophenoxyacetic acid

### Results

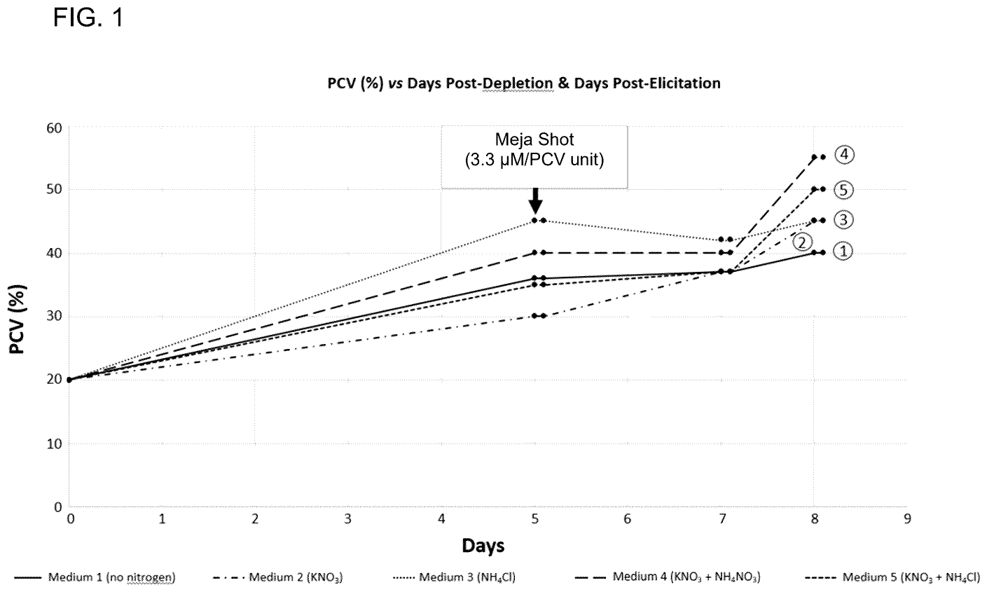

Results are presented in FIG. 1. Before elicitation, the medium providing the best conditions for growth was Medium 3 (containing NH₄Cl as the sole source of nitrogen). However, shortly after elicitation with Meja, the cells died. Before elicitation, the medium providing the lowest growth was Medium 2 (containing KNO₃ as the sole source of nitrogen). The absence of any nitrogen source (Medium 1) did not prevent the cells from growing.

Only the cells which grew in Medium 1 (no nitrogen source), prior to elicitation, were able to produce saponins (the volumetric productivity of QS-18 reached about 5 mg/L, as measured 4 days after elicitation) (data not shown).

### 3.2 Experiment 2—QS-18 Volumetric Productivity (No Nitrogen Source During Nitrogen Depletion)

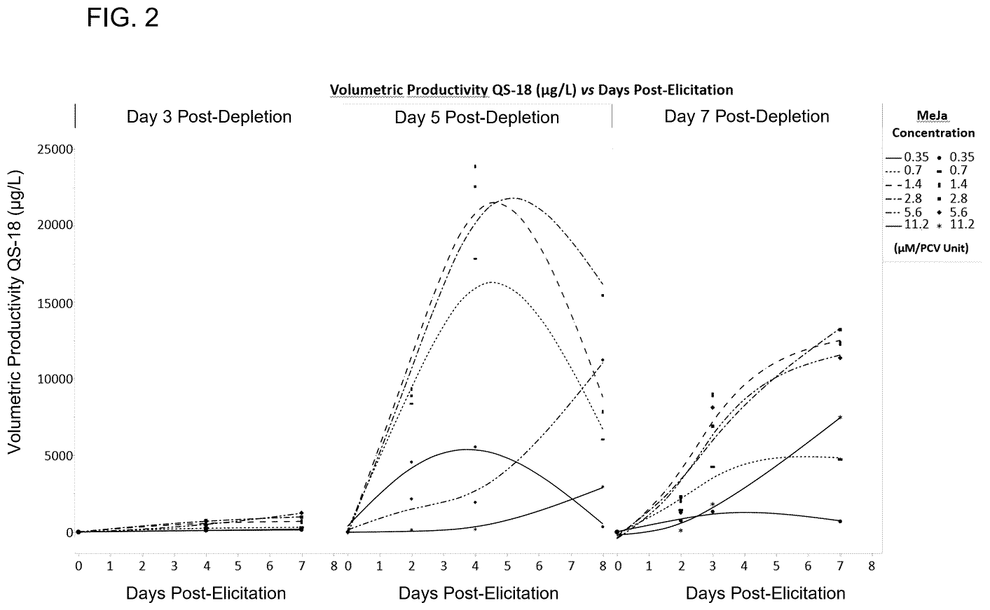

The above Medium 1 (no nitrogen source) was then selected to test different elicitation conditions (concentration and duration). 5 separate cultures of the cell line CMC40B6, wherein 10% of the cell biomass (PCV) was seeded in Medium 4, were grown until the PCV reached about 30%. At this point in time (Day 0), the culture medium of all cultures was replaced with Medium 1. At D3, D5 and D7, the PCV % of each culture was measured and MeJa was added directly to the culture media at varying concentrations (0.35, 0.7, 1.4, 2.8, 5.6 or 11.2 μM/PCV %). 2 days, 4 days and 7 days post-elicitation (i.e. after Meja was added), as well as before adding MeJa, cells were harvested and disrupted (as described in Example 4), and the saponin content in the plant cell extracts was measured (as described in Example 5). QS-18 volumetric productivity was looked at. The results are presented in FIG. 2. The data (not shown) were obtained as described for the Experiment 4 below in Table 2.

The presence of QS-21 and QS-17 saponins was also looked at and confirmed for the above condition: 5 days of nitrogen depletion, followed by a 4-day elicitation with 2.8 μM/PCV %. The results are presented in FIG. 7.

### Results

Irrespective of the MeJA concentration used, the best productivity is reached when the plant cells were depleted for 5 days (more than 20 mg/L). While 3 days of depletion produced very little of QS-18, 7 days of depletion still provided good QS-18 productivity (more than 10 mg/L). At 5 days post-depletion, QS-18 productivity reached a peak between 2 and 7 days post-elicitation, with the highest productivity being achieved around 4 to 5 days (irrespective of the Meja concentration). At 5 days post-depletion, the concentration of MeJa giving the highest productivity ranges between 0.7 and 2.8 μM/PCV %. At 7 days post-depletion, the concentration of MeJa giving the highest productivity ranges between 1.4 and 5.6 μM/PCV unit (see FIG. 2).

Moreover, the chromatogram shown in FIG. 7 indicates that QS-17 saponins and QS-21 saponins were produced as well, after a 5-day nitrogen depletion, followed by a 4-day elicitation with 2.8 μM/PCV %, as reflected by the presence of peaks at retention time corresponding to the respective standards.

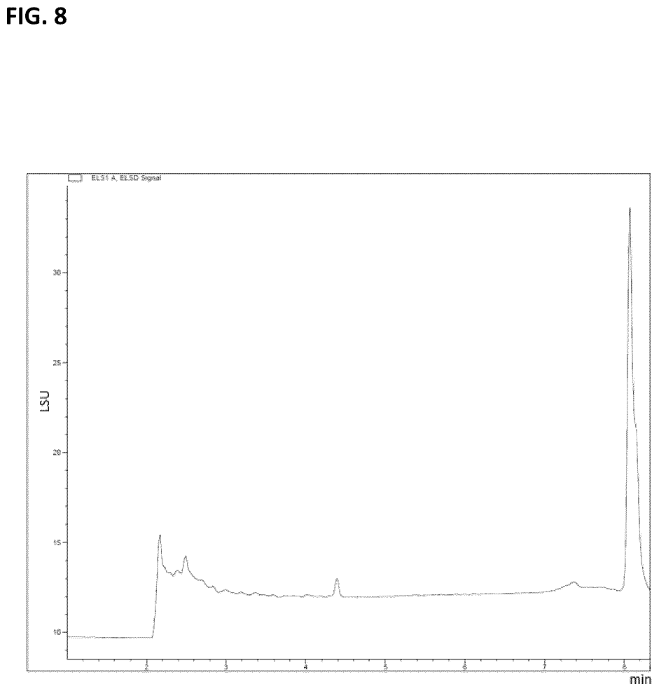

The chromatogram shown on FIG. 8 indicates that, in this experiment, a 5-day nitrogen depletion in the absence of any elicitation is not sufficient to achieve a detectable level of saponin production, as reflected by the absence of peaks at the expected retention time corresponding to the respective standards.

### 3.3 Experiment 3—QS-18 Volumetric Productivity (No Nitrogen Source During Nitrogen Depletion)

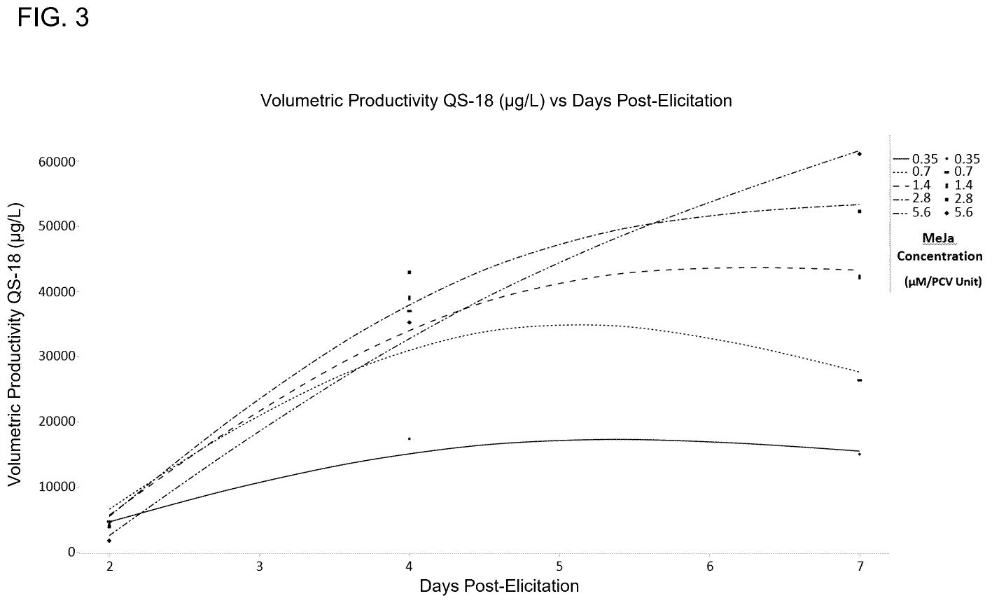

A second suspension cell line (CMC16B) (established as described in Example 1 and Example 2) was tested. 5 different cultures of this suspension cell line were grown in parallel in Medium 4, until the PCV of the cultures reached 30%. At this point in time (Day 0), the culture medium of all cultures was removed and replaced with Medium 1 (no source of nitrogen). 5 days later, at Day 5, the PCV % of each culture was measured and different MeJa concentrations were added directly to the culture medium of each culture (0.35, 0.7, 1.4, 2.8, or 5.6/PCV %). 2 days, 4 days and 7 days post-elicitation (i.e. after Meja was added), cells were harvested and disrupted (as described in Example 4), and the saponin content in the plant cell extracts was measured (as described in Example 5). QS-18 volumetric productivity was looked at. The results are presented in FIG. 3. The data (not shown) were obtained as described for the Experiment 4 below in Table 2.

### Results

Irrespective of the MeJa concentration used, a maximum productivity is obtained around 4 days after Meja was added (i.e. 4 days post-elicitation), with the highest productivity (about 40 mg/L) obtained with a 2.8 μM/PCV % MeJa concentration.

### 3.4 Experiment 4—QS-18 Volumetric Productivity (No Nitrogen Source During Nitrogen Depletion)

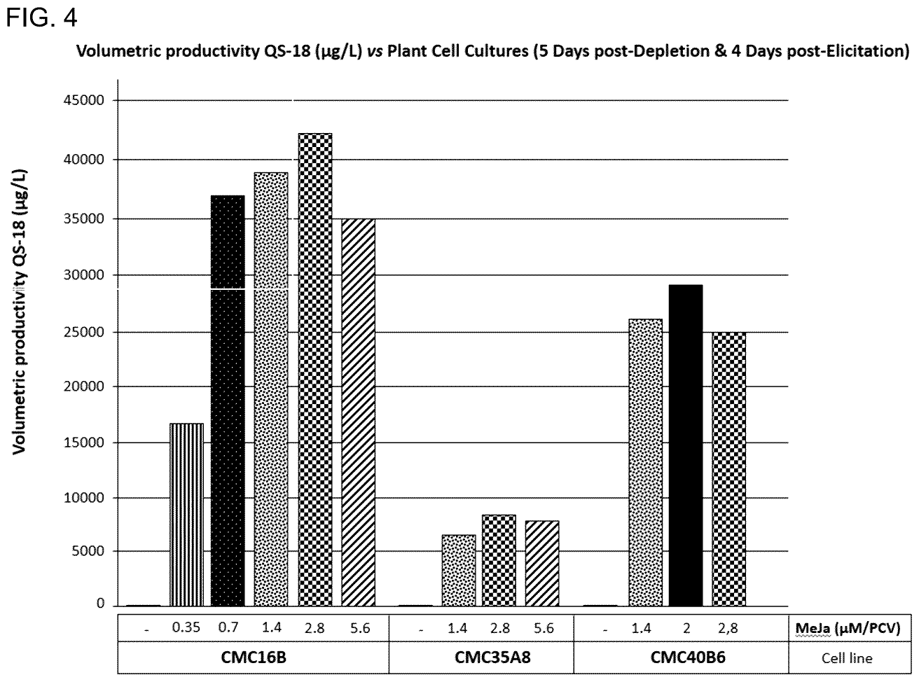

3 different suspension cell lines (CMC16B, CMC40B6 and CMC35A8) (established as described in Example 1 and Example 2) were tested. CMC16B was used at passage P19, CMC40B6 was used at passage P39 and CMC35A8 was used at passage P14. 5 separate cultures of each suspension cell line were grown in Medium 4, until the PCV of the cultures reached 30%. At this point in time (Day 0), the culture medium of all cultures was removed and replaced with Medium 1 (no source of nitrogen). 5 days later, at Day 5, the PCV % of each culture was measured and different MeJa concentrations were added directly to the culture medium of each culture of each cell line (0.35, 0.7, 1.4, 2, 2.8, or 5.6 μM/PCV %, as applicable and summarized in Table 2 and Table 3 below). 4 days post-elicitation (i.e. after Meja was added), cells were harvested and disrupted (as described in Example 4), and the saponin content in the plant cell extracts was measured (as described in Example 5). QS-18 volumetric productivity was looked at. The data are shown in Table 2 and Table 3 below and the results presented in the form of a graph in FIG. 4.

TABLE 2
QS-18 volumetric productivity (μg/L) for CMC16B (see last column)

| | | | Sample | QS-18 | QS-18 | | Total | QS-18 | QS-18 | |
|---|---|---|---|---|---|---|---|---|---|---|
| | | | (20 μl) | Std | Std | Cell | extr. | Std inj. | (μg/g | |
| | MeJa | PCV | peak | peak | conc. | FW² | vol. | vol. | Cell | QS-18 |
| Cell line | (μg/PCV) | (%) | area | area | (μg/ml) | (g) | (ml) | (μl) | FW²) | (μg/L) |
| CMC16B | — | 30 | ND¹ | 814.07 | 50 | 1.2025 | 1.8 | 10 | — | — |
| P19 | 0.35 | 37.5 | 1058.58 | 814.07 | 50 | 1.5752 | 2.25 | 10 | 46.44 | 17413.26 |
| | 0.7 | 37.5 | 1997.97 | 814.07 | 50 | 1.4003 | 2.25 | 10 | 98.59 | 36970.89 |
| | 1.4 | 35 | 2171.68 | 814.07 | 50 | 1.2573 | 2.1 | 10 | 111.39 | 38987.24 |
| | 2.8 | 35 | 2617.46 | 814.07 | 50 | 1.3768 | 2.1 | 10 | 122.60 | 42911.61 |
| | 5.6 | 35 | 2070.39 | 814.07 | 50 | 1.3267 | 2.1 | 10 | 100.64 | 35224.52 |

¹“ND” is for Non detectable
²“FW” is for Fresh Weight

Calculations are as follows (assuming the “Cell culture density” to be 1000 g/L):

$$QS - 18 ( µg \text{/Cell} FW ) = Sample peak area \times \frac{QS - 18 Std conc . ( µg / ml )}{QS - 18 Std peak area} \times \frac{QS - 18 Std inj . vol . ( µL )}{Sample inj . vol . ( µL )} \times \frac{Total extr . vol . ( ml )}{Cell FW ( g )} \\ QS - 18 ( µg \text{/L} ) = QS - 18 ( µg \text{/Cell} FW ) \times \frac{P C V ( \% )}{100} \times Cell culture density ( \text{g/L} )$$

TABLE 3
QS-18 volumetric productivity (μg/L) for CMC35A8 and CMC40B6 (see last column)

| | | | Sample | | Total | | | QS-18 | |
|---|---|---|---|---|---|---|---|---|---|
| | | | (20 μl) | Cell | extr. | Slope | Intercept | (μg/g | |
| | MeJa | PCV | peak | FW² | vol. | Std | Std | cell | QS-18 |
| Cell line | (μg/PCV) | (%) | area | (g) | (ml) | curve | curve | FW²) | (μg/L) |
| CMC35A8 | — | 41 | ND¹ | 1.4722 | 2.46 | 3.61 | −602.17 | — | — |
| P14 | 1.4 | 45 | 15.68 | 1.4316 | 2.7 | 3.61 | −602.17 | 16.13 | 7257.88 |
| | 2.8 | 45 | 5.85 | 1.281 | 2.7 | 3.61 | −602.17 | 17.74 | 7982.10 |
| | 5.6 | 45 | 14.54 | 1.3306 | 2.7 | 3.61 | −602.17 | 17.32 | 7794.39 |
| CMC40B6 | — | 57 | ND | 2.0322 | 3.4 | 3.61 | −602.17 | — | — |
| P39 | 1.4 | 40 | 960.93 | 1.3804 | 2.4 | 2.41 | −614.55 | 56.72 | 22687.14 |
| | 1.4 | 33 | 1763.01 | 1.1153 | 2 | 2.41 | −614.55 | 88.28 | 29427.23 |
| | 2 | 33 | 1954.36 | 1.1383 | 2 | 2.41 | −614.55 | 93.46 | 31153.14 |
| | 2 | 40 | 1184.27 | 1.2957 | 2.4 | 2.41 | −614.55 | 68.99 | 27596.58 |
| | 2.8 | 33 | 1527.55 | 1.2654 | 2 | 2.41 | −614.55 | 70.10 | 23368.00 |
| | 2.8 | 47 | 958.74 | 1.5754 | 2.8 | 2.41 | −614.55 | 57.90 | 27019.88 |

¹“ND” is for Non-detectable
²“FW” is for Fresh Weight

Calculations are as follows (assuming the “Cell culture density” to be 1000 g/L):

$$QS - 18 ( µg \text{/Cell} FW ) = \frac{Sample peak area - Intercept Std curve}{Slope Std curve} \times \frac{1}{Sample inj . vol . ( µL )} \times \frac{Total extr . vol . ( ml )}{Cell FW ( g )} \\ QS - 18 ( µg \text{/L} ) = QS - 18 ( µg / Cell FW ) \times \frac{P C V ( \% )}{100} \times Cell culture density ( \text{g/L} )$$

### Results

FIG. 4 indicates some cell line variability, likely to reflect some inherent variability in the ability of different cell lines to synthesize saponins. However, with all 3 cell lines, nitrogen depletion, followed by elicitation in accordance with the method of the invention resulted in saponin production. Moreover, the effectiveness and reproducibility of the method of the invention is confirmed by the data obtained for the cell lines CMC40B6 and CMC16B, as the level of QS-18 volumetric productivity is within the same the range as what was obtained in Experiments 2 and 3 and shown in FIGS. 2 and 3.

### 3.5 Experiment 5—QS-21 Volumetric Productivity (No Nitrogen Source During Nitrogen Depletion)

The same suspension cell lines as used in the above Experiment 4 (CMC16B, CMC40B6 and CMC35A8) were tested. CMC16B was used at passage P19, CMC40B6 was used at passage P27 and CMC35A8 was used at passage P15. A parent culture of each suspension cell line grown in Medium 4 was split into 5 separate cultures by centrifuging the cells of the parent culture and resuspending them into Medium 1 (no source of nitrogen) at a PCV between 20-25% (this is Day 0). 5 days later, at Day 5, the PCV % of each culture was measured and different MeJa concentrations were added directly to the culture medium of each culture of each cell line (0.35, 0.7, 1.4, 2.8, or 5.6 μM/PCV %) (as applicable and summarized in Table 4).

### Glucose and Phosphate (PO₄) Feed Conditions During the Nitrogen Depletion and Elicitation Phases:

The minimum target for glucose level and phosphate (PO₄) level during both the nitrogen depletion phase and the elicitation phase was 15 mM and 0.6 mM, respectively. Glucose level and phosphate (PO₄) level were measured at the following time points: (i) before starting the nitrogen depletion, 2 days and 5 days post-depletion; and (ii) before starting the elicitation, 2 days, 4 days and 7 days post-elicitation, or 8 days post-elicitation (as applicable). In case the measured level of glucose was lower than 15 mM, the culture medium was fed with a 60 mM glucose solution. In case the measured level of phosphate (PO₄) level was lower than 0.6 mM, the culture medium was fed with a 2.5 mM phosphate solution.

### Note

During this experiment, phosphate (PO₄) starvation was observed during the nitrogen depletion phase and/or the elicitation phase for all 3 cell lines: (i) at D5 post-depletion and D2/D4/D8 post-elicitation for CMC40B6; (ii) at D2/D5 post-depletion and D4/D7 post-elicitation for CMC16B; and (iii) at D2/D5 post-depletion for CMC35A8. By “starvation”, it is meant that the phosphate (PO₄) level measured at the indicated days was below the limit of detection.

7 days post-elicitation (CMC16B and CMC35A8), or 8 days post-elicitation (CMC40B6), i.e. after Meja was added, cells were harvested and disrupted as described below.

### Saponin Extraction

Before harvesting the cells, the PCV of the cell cultures was measured. Samples of the cell culture were collected and centrifuged. The supernatant was discarded and the cell pellet dried in a lyophilizer, and weighed (“Cell FW”). Once dried, 1 mL (“total extraction volume”) of 80% methanol was added to the cell pellet. Extraction was performed either by vortexing the samples at 2500 rpm for 90 min or by ball milling. The mixture was then centrifuged for 5 min at 1000 g. After centrifugation, 1 μL of the recovered supernatant was analyzed according to the method described in Example 6.

QS-21 volumetric productivity was looked at and the saponin content in the plant cell extracts was measured (as described in Example 6).

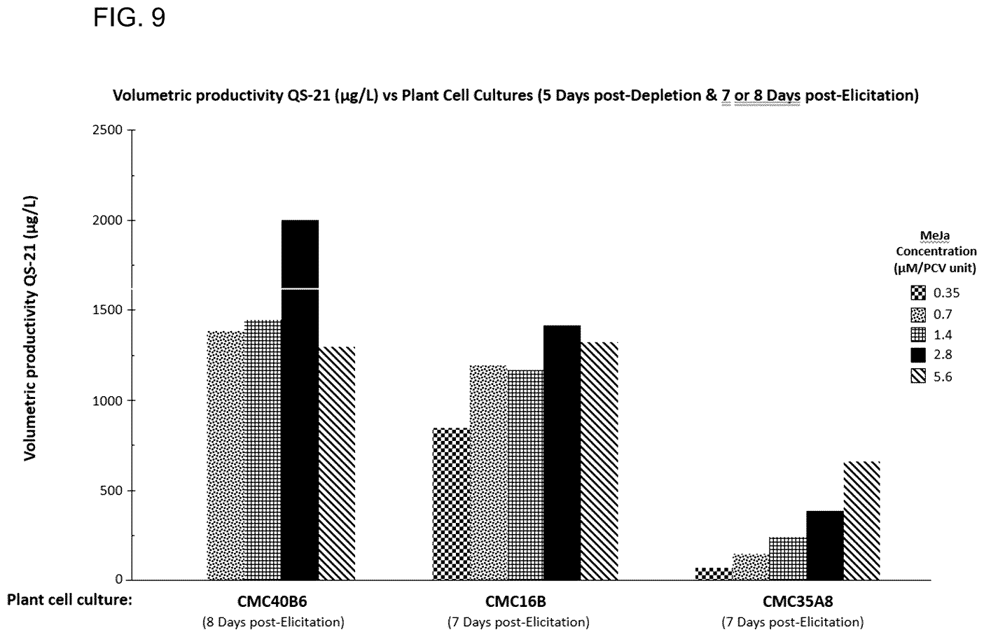

The data are shown in Table 4 and the results presented in the form of a graph in FIG. 9.

TABLE 4
QS-21 volumetric productivity (μg/L) (see last column)

| | | | Sample | QS-21 | QS-21 | | Total | QS-21 | | |
|---|---|---|---|---|---|---|---|---|---|---|
| | | | (1 μl) | Std | Std | Cell | extr. | Std inj. | QS-21 | |
| | MeJa | PCV | peak | peak | Conc. | FW₁ | vol. | vol | (μg/g | QS-21. |
| Cell line | (μM/PCV) | (%) | area | area | (μg/ml) | (g) | (ml) | (μl) | FW)¹ | (μg/L)² |
| CMC16B | 0.35 | 46 | 805 | 3910 | 10 | 1.1204 | 1 | 1 | 1.8 | 845.465 |
| P19 | 0.7 | 45 | 1102 | 3910 | 10 | 1.0633 | 1 | 1 | 2.7 | 1192.565 |
| | 1.4 | 40 | 1064 | 3910 | 10 | 0.9299 | 1 | 1 | 2.9 | 1170.674 |
| | 2.8 | 40 | 1255 | 3910 | 10 | 0.9044 | 1 | 1 | 3.5 | 1418.991 |
| | 5.6 | 40 | 1178 | 3910 | 10 | 0.9086 | 1 | 1 | 3.3 | 1326.191 |
| CMC35A8 | 0.35 | 38 | 88 | 4138 | 10 | 1.1168 | 1 | 1 | 0.19 | 72.018 |
| P15 | 0.7 | 40 | 160.5 | 4138 | 10 | 1.0453 | 1 | 1 | 0.375 | 148.350 |
| | 1.4 | 45 | 254 | 4138 | 10 | 1.1426 | 1 | 1 | 0.54 | 242.388 |
| | 2.8 | 46 | 374.5 | 4369 | 10 | 1.0185 | 1 | 1 | 0.835 | 385.710 |
| | 5.6 | 43 | 797.5 | 4369 | 10 | 1.1688 | 1 | 1 | 1.54 | 663.465 |
| CMC40B6 | 0.7 | 43 | 825 | 2570 | 10 | 0.9848 | 1 | 1 | 3.260 | 1385.315 |
| P27 | 1.4 | 45 | 933 | 2570 | 10 | 1.1277 | 1 | 1 | 3.220 | 1448.970 |
| | 2.8 | 40 | 1347 | 2570 | 10 | 1.0467 | 1 | 1 | 5.007 | 2002.940 |
| | 5.6 | 30 | 988 | 2570 | 10 | 0.8900 | 1 | 1 | 4.320 | 1295.992 |

¹“FW” is for Fresh Weight

Calculations are as follows (assuming the “Cell culture density” to be 1000 g/L):

$$QS - 21 ( µg \text{/Cell} FW ) = Sample peak area \times \frac{QS - 21 Std conc . ( µg / ml )}{QS - 21 Std peak area} \times \frac{QS - 21 Std inj . vol . ( µL )}{Sample inj . vol . ( µL )} \times \frac{Total extr . vol . ( ml )}{Cell FW ( g )} \\ QS - 21 ( µg \text{/L} ) = QS - 21 ( µg \text{/Cell} FW ) \times \frac{P C V ( \% )}{100} \times Cell culture density ( \text{g/L} )$$

### Results

Like for QS-18 volumetric productivity (FIG. 8), some cell line variability (likely to reflect some inherent variability in the ability of different cell lines to synthesize saponins) was observed (FIG. 9). However, with all 3 cell lines, nitrogen depletion, followed by elicitation in accordance with the method of the invention resulted in QS-21 saponin production.

### 3.6 Experiment 6—QS-21 Volumetric Productivity (No Nitrogen Source During Nitrogen Depletion)

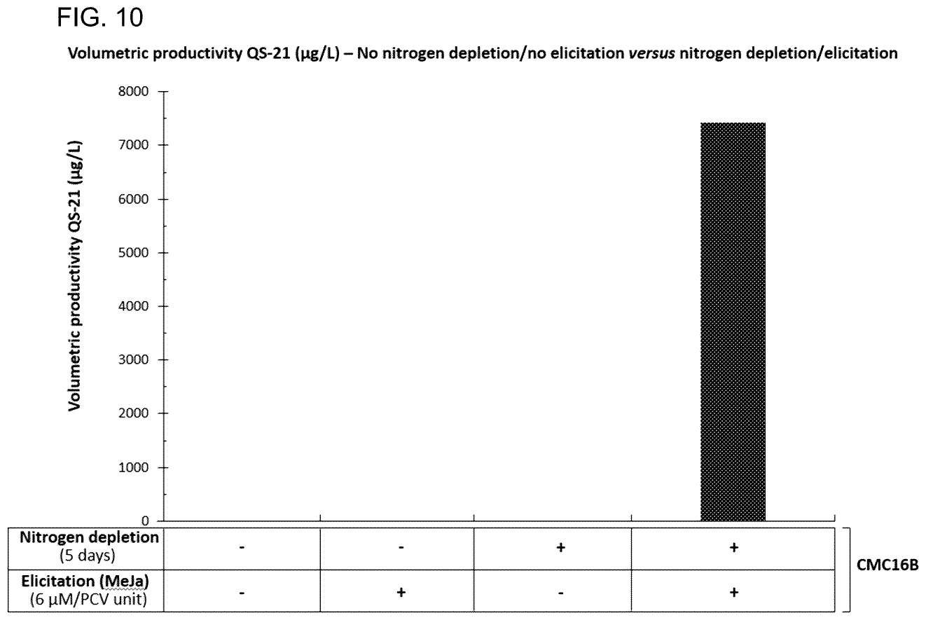

The same suspension cell line (CMC16B) as used in the above Experiments 4 and 5 was tested, at passage P40. A parent culture of CMC16B grown in Medium 6 was split into 4 separate cultures by centrifuging the cells of the parent culture and resuspending them at a PCV between 20-25% (this is Day 0). 2 cultures were resuspended in Medium 1 (no source of nitrogen), 2 cultures were resuspended in Medium 6 (comprising nitrogen). 5 days later, at Day 5, all 4 cultures were either left untreated (i.e. no Meja addition) or 6 μM/PCV % of MeJa was added directly to the culture medium (as summarized in Table 5 below and indicated in FIG. 10).

### Glucose and Phosphate (PO₄) Feed Conditions During the Nitrogen Depletion and Elicitation Phases:

The minimum target for glucose level and phosphate (PO₄) level during the duration of both the nitrogen depletion phase and the elicitation phase was 15 mM and 1.5 mM, respectively. Glucose level and phosphate (PO₄) level were measured at the following time points: (i) before starting the nitrogen depletion, 2 days and 5 days post-depletion; and (ii) before starting the elicitation, 2 days, 4 days and 7 days post-elicitation. In case the measured level of glucose was lower than 15 mM, the culture medium was fed with a 60 mM glucose solution In case the measured level of phosphate (PO₄) level was lower than 1.5 mM, the culture medium was fed with a 5 mM phosphate solution. No phosphate (PO₄) starvation was observed during this experiment.

7 days post-elicitation (i.e. after Meja was added), both untreated cells and Meja-treated cells were harvested and disrupted (as described in Experiment 5), and the saponin content in the plant cell extracts was measured (as described in Example 6). The measurement was done in duplicates or triplicates, as applicable. QS-21 volumetric productivity was looked at. The data are shown in Table 5 and the results presented in the form of a graph in FIG. 10 (each bar representing the averaged volumetric productivity).

TABLE 5
QS-21 volumetric productivity (μg/L) (see last column)

| | | | | QS-21 | | | | | | | |
|---|---|---|---|---|---|---|---|---|---|---|---|
| | | | Sample | Std | QS-21 | | Total | | QS-21 | | |
| | | | (1 μl) | (1 μl) | Std | Cell | extr. | | (μg/g | | |
| | Meja | PCV | peak | peak | Conc. | FW¹ | vol. | | Cell | QS-21 | Sample |
| Nitrogen | (μM/PCV) | (%) | area | area | (μg/ml) | (g) | (ml) | Dil.² | FW¹) | (μg/L) | No. |
| − | − | 60 | 0 | 6095 | 10 | 1.9834 | 1 | 1 | 0 | 0 | 1 |
| | | 60 | 0 | 6095 | 10 | 1.3280 | 1 | 1 | 0 | 0 | 2 |
| | | 60 | 0 | 6095 | 10 | 1.9560 | 1 | 1 | 0 | 0 | 3 |
| − | 6 | 45 | 0 | 6095 | 10 | 0.9678 | 1 | 1 | 0 | 0 | 4 |
| | | 45 | 0 | 6095 | 10 | 1.7913 | 1 | 1 | 0 | 0 | 6 |
| + | − | 55 | 0 | 6095 | 10 | 1.8078 | 1 | 1 | 0 | 0 | 7 |
| | | 60 | 0 | 6095 | 10 | 1.9581 | 1 | 1 | 0 | 0 | 8 |
| | | 60 | 0 | 6095 | 10 | 1.5089 | 1 | 1 | 0 | 0 | 9 |
| + | 6 | 45 | 1825 | 5377 | 10 | 2.0113 | 1 | 0.1 | 16.878 | 7595 | 10 |
| | | 45 | 1500 | 5377 | 10 | 1.8871 | 1 | 0.1 | 14.785 | 6653 | 11 |
| | | 45 | 1785 | 5377 | 10 | 1.8638 | 1 | 0.1 | 17.812 | 8015 | 12 |

¹“FW” is for Fresh Weight
²“Dil.” is for dilution of the extracted sample (prior injection)

Calculations are as follows (assuming the “Cell culture density” to be 1000 g/L):

$$QS - 21 ( µg \text{/Cell} FW ) = Sample peak area \times \frac{QS - 21 Std conc . ( µg / ml )}{QS - 21 Std peak area} \times \frac{QS - 21 Std inj . vol . ( µL )}{Sample inj . vol . ( µL )} \times \frac{Total extr . vol . ( ml )}{Cell FW ( g )} \\ QS - 21 ( µg \text{/L} ) = QS - 21 ( µg \text{/Cell} FW ) \times \frac{P C V ( \% )}{100} \times Cell culture density ( \text{g/L} )$$

### Results

While in the absence of nitrogen depletion and/or elicitation, no production of QS-21 saponins was observed, a nitrogen depletion phase followed by elicitation results in the production of QS-21 saponins, as illustrated in FIG. 10.

### 3.7 Experiment 7-QS-21 Volumetric Productivity (No Nitrogen Source During Nitrogen Depletion)

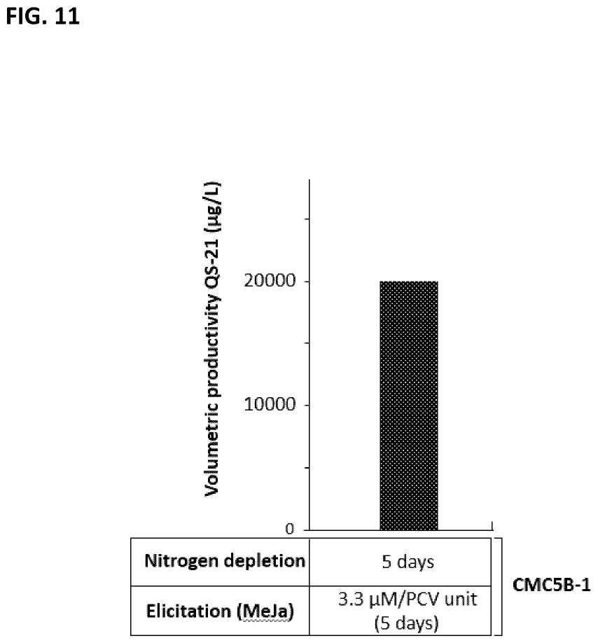

A fourth suspension cell line (CMC5B-1) (established as described in Example 1 and Example 2) was tested at passage P11. A parent culture of CMC5B-1 grown in Medium 4 was centrifuged and resuspended into Medium 1 (no source of nitrogen) at a PCV between 20-25% (this is Day 0). 5 days later, at Day 5, the PCV % of each culture was measured and 3.3 μM/PCV % MeJa was added directly to the culture medium (i.e. after Meja was added) for 5 days. Cells were then harvested and disrupted as described below, and the saponin content in the plant cell extract was measured (as described in Example 6). QS-21 volumetric productivity was looked at. The data are shown in Table 6 and the results are presented in the form of a graph in FIG. 11.

### Extraction of Saponins

Before harvesting the cells, the PCV of the cell culture was measured. 1 ml of the cell culture (“Vol. PCC”) was centrifuged for 10 minutes at 4000 rpm. The supernatant was discarded, while the cell pellet was frozen at −70° C. for 24 h to cause lysis of the cells. The defrosted sample cell pellet was then diluted with 4 ml methanol, and vortexed for 30 seconds prior to centrifugation. The supernatant was recovered and diluted. 1 μL of the diluted sample was then used for analyzing the content of saponins according to the method described in Example 6.

TABLE 6
QS-21 volumetric productivity (μg/L) (see last column)

| | | | QS-21 | | | | | | |
|---|---|---|---|---|---|---|---|---|---|
| | | Sample | Std | QS-21 | | Total | | QS-21 | |
| | | (1 μl) | (1 μL) | Std | Vol. | extr. | | Sample | |
| | PCV | peak | peak | Conc. | PCC | vol. | | Conc. | QS-21 |
| Cell line | (%) | area | area | (μg/ml) | (mL) | (mL) | Dilution² | (μg/mL) | (μg/L) |
| CMC5B-1 | 35 | 2969 | 7808 | 10 | 1 | 4 | 0.333 | 11.41 | 20000 |

¹ “FW” is for Fresh Weight
²Dilution of the extracted sample (prior injection)

Calculations are as follows:

$$QS - 21 concentration sample ( µg \text{/mL} ) = Sample peak area \times \frac{QS - 21 Std conc . ( µg / ml )}{QS - 21 Std peak area} \times \frac{Q - 21 Std inj . vol . ( µL )}{Sample inj . vol . ( µL )} \times \frac{1}{Dilution} \\ QS - 21 ( µg \text{/L} ) = QS - 21 concentration sample ( µg \text{/mL} ) \times \frac{P C V ( \% )}{100} \times \frac{Total extr . vol . ( ml ) + vol . PCC ( mL )}{Vol . PCC ( mL )} \times 1000$$

### Results

As for Experiment 5 and Experiment 6, the production of QS-21 saponins was observed after a nitrogen depletion phase followed by elicitation (as illustrated in FIG. 11).

### 3.8 Experiment 8—QS-7 Production (No Nitrogen Source During Nitrogen Depletion)

Samples No. 2 and No. 12 obtained in Experiment 6 were also tested for the presence of QS-7 saponins. As detailed in Experiment 6, Sample No. 2 and Sample No. 12 are representative of conditions of “no nitrogen depletion”/“no elicitation” and “nitrogen depletion/elicitation”, respectively.

The presence of QS-7 was detected by UPLC/MS, using the following parameters:

| Instrument | | | |
|---|---|---|---|
| UPLC-MS | Waters Acquity H class UPLC coupled to a | | |
| | Waters Vion QTOF mass spec with ion | | |
| | mobility. Operated using UNIFI software | | |
| UPLC Conditions | | | |
| Column | Waters BEH C18 1.7 um 2.1 × 100 mm | | |
| Temperature | 40° C. ± 3° C. | | |
| Flow Rate | 0.5 mL/min | | |
| Gradient Curve | 6 | | |
| Injection Volume | 5 μL | | |
| Autosampler Temperature | 10° C. ± 3° C. | | |
| Run Time | 120 mins | | |
| Post-Inject Wash | 50% methanol | | |
| Mobile Phase A | 0.1% Formic Acid in Water | | |
| Mobile Phase B | 0.1% Formic Acid in Acetonitrile | | |
| Syringe Wash Solvent | 50% Methanol | | |
| Seal Wash | 50% Methanol | | |
| Purge Solvent | 50% Acetonitrile | | |
| Gradient | Time | % A | % B |
| | 0 | 75 | 25 |
| | 0.75 | 75 | 25 |
| | 80 | 50 | 50 |
| | 90 | 5 | 95 |
| | 110 | 5 | 95 |
| | 111 | 75 | 25 |
| | 120 | 75 | 25 |

Mass Spec Conditions

| | |
|---|---|
| Source Type | ESI |
| Polarity | Negative |
| Analyzer Mode | Sensitivity |
| Transition mode | Standard |
| Capillary Voltage | 1.5 Kv |
| Sample Cone Voltage | 40 V |
| Source Offset Voltage | 80 V |
| Source Temperature | 120 deg C. |
| Desolvation Temperature | 450 deg C. |
| Cone Gas flow | 50 L/h |
| Desolvation Gas flow | 1000 L/h |
| Collision Gas | Nitrogen |
| Mode | MSe with ion mobility |
| Scan | 300-4000 |
| Scan Time | 0.3 |
| Collision energy | Low energy 6 V |
| | High energy ramp 10 V to 45 V |

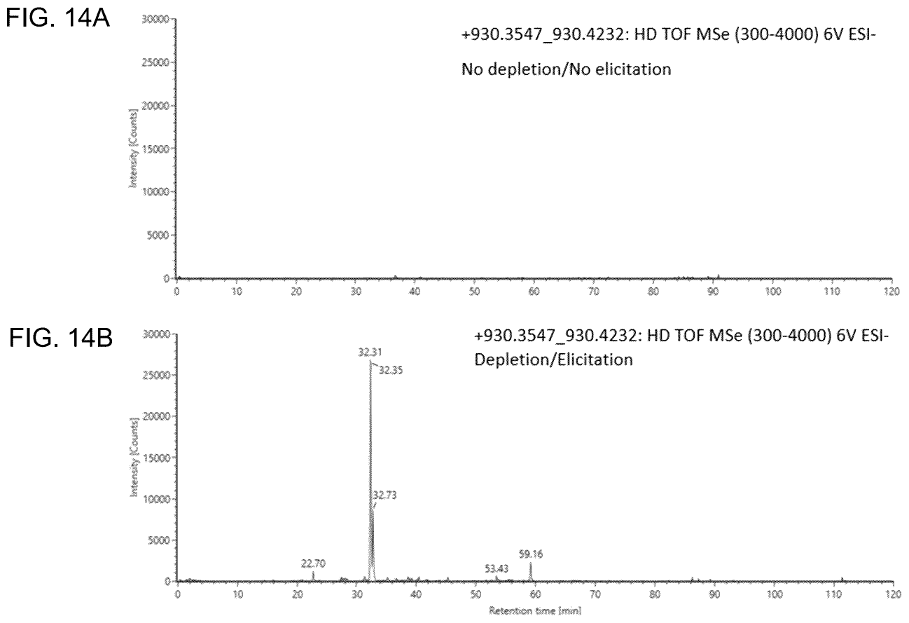

The presence of QS-7 saponins in the plant cell extract was determined by extracting the double charged [M−2H]²⁻ (1861.78-1)/2=930.39. The mono charged [M-H]⁻ 1861.78 ions corresponding to QS-7 1862. Chromatograms representative of conditions of “no nitrogen depletion”/“no elicitation” and “nitrogen depletion/elicitation” are provided in FIGS. 14A and 14B.

### Results

While in the absence of both nitrogen depletion and elicitation, QS-7 1862 was not detected, QS-7 1862 becomes detectable following both nitrogen depletion and elicitation (see FIG. 14A and FIG. 14B, respectively). This indicates that QS-7 saponins are produced as well, when using methods of the invention.

### 3.9 Experiment 9—QS-18 Volumetric Productivity (Reduced Concentration of Nitrogen Source During Nitrogen Depletion)

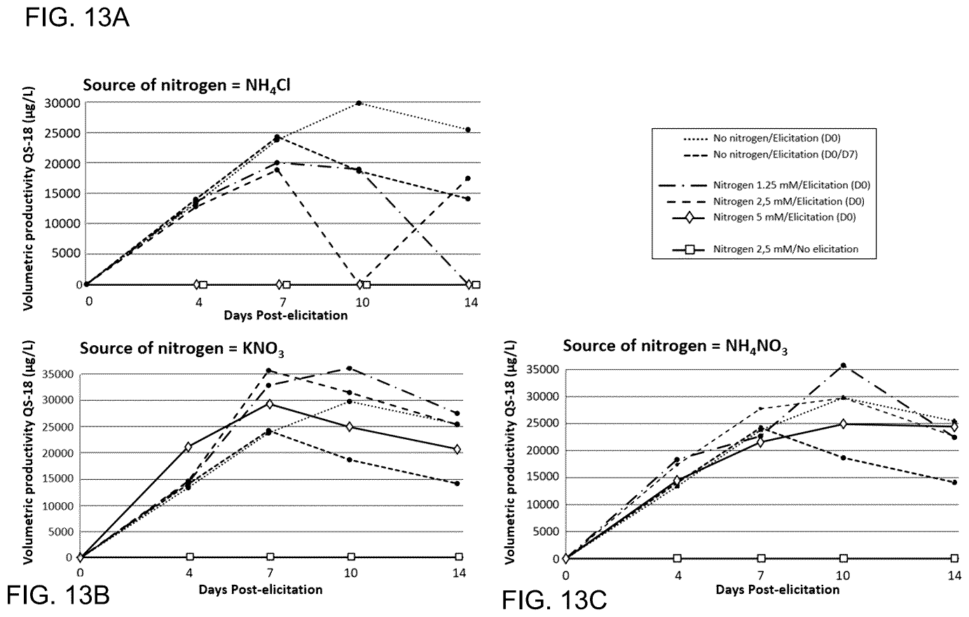

The same suspension cell line (CMC16B) as used in the above Experiments 4, 5 and 6 was tested, at passage P47. A parent culture of CMC16B grown in Medium 6 was split into 13 separate cultures by centrifuging the cells of the parent culture and resuspending them at a PCV of about 20% (this is Day 0) in either Medium 1 (i.e. no nitrogen source), or Medium 1 supplemented with 1.25 mM, 2.5 mM or 5 mM NH₄Cl, NH₄NO₃ or KNO₃ (as indicated in FIGS. 13A-13C). 5 days later, at Day 5, cultures were either left untreated (i.e. no Meja addition) or 8 μM/PCV % of MeJa was added directly to the culture medium (as indicated in FIG. 13). 4 days, 7 days, 10 days and 14 days post-elicitation, cells were harvested and disrupted (as described in Example 4), and the saponin content in the plant cell extracts was measured (as described in Example 5). QS-18 volumetric productivity was looked at. The data are shown in Table 7, Table 8 and Table 9, and the results presented in FIGS. 13A-13C.

TABLE 7
QS-18 volumetric productivity (μg/L) (see last column)/4 days post-elicitation

| | | | Sample | | Total | | | QS-18 | |
|---|---|---|---|---|---|---|---|---|---|
| | | | (20 μl) | Cell | extr. | Slope | Intercept | (μg/g | |
| | MeJa | PCV | peak | FW | vol. | Std | Std | cell | QS-18 |
| Nitrogen | (μM/PCV) | (%) | area | (g) | (ml) | curve | curve | FW²) | (μg/L) |
| — | 8* | 57.1 | 106.2 | 1.6688 | 3.3 | 2.57 | −493.55 | 23.3 | 13319.8 |
| — | 8** | 55.2 | 157.9 | 1.6047 | 3.2 | 2.57 | −493.55 | 25.3 | 13992.8 |
| NH₄Cl | 8 | 57.0 | 117.3 | 1.656 | 3.3 | 2.57 | −493.55 | 23.7 | 13540.4 |
| 1.25 mM | | | | | | | | | |
| NH₄Cl | 8 | 61.0 | 43.7 | 1.6735 | 3.3 | 2.57 | −493.55 | 20.9 | 12737.1 |
| 2.5 mM | | | | | | | | | |
| NH₄Cl | 8 | 65.5 | ND¹ | 1.8283 | 3.7 | 2.57 | −493.55 | ND¹ | ND¹ |
| 5 mM | | | | | | | | | |
| NH₄Cl | — | 78.3 | ND¹ | 2.1836 | 4.4 | 2.57 | −493.55 | ND¹ | ND¹ |
| 2.5 mM | | | | | | | | | |
| KNO₃ | 8 | 52.9 | 205.1 | 1.5661 | 3.1 | 2.57 | −493.55 | 27.2 | 14373.3 |
| 1.25 mM | | | | | | | | | |
| KNO₃ | 8 | 50.2 | 256.6 | 1.4353 | 2.9 | 2.57 | −493.55 | 29.2 | 14635.2 |
| 2.5 mM | | | | | | | | | |
| KNO₃ | 8 | 78.5 | 199.3 | 2.1876 | 4.4 | 2.57 | −493.55 | 26.9 | 21133.3 |
| 5 mM | | | | | | | | | |
| KNO₃ | — | 52.7 | ND¹ | 1.5843 | 3.2 | 2.57 | −493.55 | ND¹ | ND¹ |
| 2.5 mM | | | | | | | | | |
| NH₄NO₃ | 8 | 55.8 | 354.4 | 1.7177 | 3.4 | 2.57 | −493.55 | 33.0 | 18399.2 |
| 1.25 mM | | | | | | | | | |
| NH₄NO₃ | 8 | 57.0 | 287.1 | 1.6082 | 3.2 | 2.57 | −493.55 | 30.4 | 17304.0 |
| 2.5 mM | | | | | | | | | |
| NH₄NO₃ | 8 | 58.4 | 144.3 | 1.7157 | 3.4 | 2.57 | −493.55 | 24.8 | 14491.3 |
| 5 mM | | | | | | | | | |
| NH₄NO₃ | — | 81.8 | ND¹ | 2.4172 | 4.8 | 2.57 | −493.55 | ND¹ | ND¹ |
| 2.5 mM | | | | | | | | | |

*Meja was added once (at D 0)
**Meja was added twice (at D 0 and D 7)
¹“ND” is for Non-detectable
²“FW” is for Fresh Weight

Calculations are as follows (assuming the “Cell culture density” to be 1000 g/L):

$$QS - 18 ( µg \text{/Cell} FW ) = \frac{Sample peak area - Intercept Std curve}{Slope Std curve} \times \frac{1}{Sample inj . vol . ( µL )} \times \frac{Total extr . vol . ( ml )}{Cell FW ( g )} \\ QS - 18 ( µg \text{/L} ) = QS - 18 ( µg \text{/Cell} FW ) \times \frac{P C V ( \% )}{100} \times Cell culture density ( \text{g/L} )$$

TABLE 8
QS-18 volumetric productivity (μg/L) (see last column)/7 days post-elicitation

| | | | Sample | | Total | | | QS-18 | |
|---|---|---|---|---|---|---|---|---|---|
| | | | (20 μl) | Cell | extr. | Slope | Intercept | (μg/g | |
| | MeJa | PCV | peak | FW | vol. | Std | Std | cell | QS-18 |
| Nitrogen | (μM/PCV) | (%) | area | (g) | (ml) | curve | curve | FW²) | (μg/L) |
| — | 8* | 65.7 | 443.7 | 1.9315 | 3.9 | 2.83 | −579.6 | 36.2 | 23769.2 |
| — | 8** | 63.2 | 504.9 | 1.8263 | 3.7 | 2.83 | −579.6 | 38.3 | 24233.1 |
| NH₄Cl | 8 | 74.2 | 185.5 | 2.251 | 4.5 | 2.83 | −579.6 | 27.0 | 20050.1 |
| 1.25 mM | | | | | | | | | |
| NH₄Cl | 8 | 78.1 | 101.9 | 2.3591 | 4.7 | 2.83 | −579.6 | 24.1 | 18805.7 |
| 2.5 mM | | | | | | | | | |
| NH₄Cl | 8 | 59.0 | ND¹ | 1.6593 | 3.3 | 2.83 | −579.6 | ND¹ | ND¹ |
| 5 mM | | | | | | | | | |
| NH₄Cl | — | 78.4 | ND¹ | 2.1146 | 4.2 | 2.83 | −579.6 | ND¹ | ND¹ |
| 2.5 mM | | | | | | | | | |
| KNO₃ | 8 | 65.1 | 848 | 1.9522 | 3.9 | 2.83 | −579.6 | 50.4 | 32853.4 |
| 1.25 mM | | | | | | | | | |
| KNO₃ | 8 | 63.7 | 1005 | 1.8196 | 3.6 | 2.83 | −579.6 | 56.0 | 35666.3 |
| 2.5 mM | | | | | | | | | |
| KNO₃ | 8 | 59.0 | 829 | 1.6897 | 3.4 | 2.83 | −579.6 | 49.8 | 29365.4 |
| 5 mM | | | | | | | | | |
| KNO₃ | — | 79.5 | ND¹ | 2.2633 | 4.5 | 2.83 | −579.6 | ND¹ | ND¹ |
| 2.5 mM | | | | | | | | | |
| NH₄NO₃ | 8 | 68.0 | 364 | 2.0758 | 4.2 | 2.83 | −579.6 | 33.3 | 22677.5 |
| 1.25 mM | | | | | | | | | |
| NH₄NO₃ | 8 | 72.1 | 511 | 2.176 | 4.4 | 2.83 | −579.6 | 38.5 | 27773.4 |
| 2.5 mM | | | | | | | | | |
| NH₄NO₃ | 8 | 77.5 | 207 | 2.4061 | 4.8 | 2.83 | −579.6 | 27.8 | 21538.5 |
| 5 mM | | | | | | | | | |
| NH₄NO₃ | — | 79.7 | ND¹ | 2.1539 | 4.3 | 2.83 | −579.6 | ND¹ | ND¹ |
| 2.5 mM | | | | | | | | | |

*Meja was added once (at D 0)
**Meja was added twice (at D 0 and D 7)
¹“ND” is for Non-detectable
²“FW” is for Fresh Weight

Calculations are as follows (assuming the “Cell culture density” to be 1000 g/L):

$$QS - 18 ( µg \text{/Cell} FW ) = \frac{Sample peak area - Intercept Std curve}{Slope Std curve} \times \frac{1}{Sample inj . vol . ( µL )} \times \frac{Total extr . vol . ( ml )}{Cell FW ( g )} \\ QS - 18 ( µg \text{/L} ) = QS - 18 ( µg \text{/Cell} FW ) \times \frac{P C V ( \% )}{100} \times Cell culture density ( \text{g/L} )$$

TABLE 9
QS-18 volumetric productivity (μg/L) (see last column)/10 days post-elicitation

| | | | Sample | | Total | | | QS-18 | |
|---|---|---|---|---|---|---|---|---|---|
| | | | (20 μl) | Cell | extr. | Slope | Intercept | (μg/g | |
| | | PCV | peak | FW | vol. | Std | Std | cell | QS-18 |
| Nitrogen | MeJa | (%) | area | (g) | (ml) | curve | curve | FW²) | (μg/L) |
| — | 8* | 73.5 | 581.6 | 2.03 | 4.1 | 2.744 | −530.78 | 40.5 | 29807.5 |
| — | 8** | 61.5 | 302.7 | 1.6906 | 3.4 | 2.744 | −530.78 | 30.4 | 18664.9 |
| NH₄Cl | 8 | 79.2 | 124.2 | 2.3645 | 4.7 | 2.744 | −530.78 | 23.9 | 18915.2 |
| 1.25 mM | | | | | | | | | |
| NH₄Cl | 8 | 81.2 | ND¹ | 2.2643 | 4.5 | 2.744 | −530.78 | ND¹ | ND¹ |
| 2.5 mM | | | | | | | | | |
| NH₄Cl | 8 | 56.7 | ND¹ | 1.6551 | 3.3 | 2.744 | −530.78 | ND¹ | ND¹ |
| 5 mM | | | | | | | | | |
| NH₄Cl | — | 67.7 | ND¹ | 1.9389 | 3.9 | 2.744 | −530.78 | ND¹ | ND¹ |
| 2.5 mM | | | | | | | | | |
| KNO₃ | 8 | 71.6 | 854 | 1.9599 | 3.9 | 2.744 | −530.78 | 50.5 | 36119.9 |
| 1.25 mM | | | | | | | | | |
| KNO₃ | 8 | 69.4 | 713 | 1.8754 | 3.8 | 2.744 | −530.78 | 45.3 | 31474.9 |
| 2.5 mM | | | | | | | | | |
| KNO₃ | 8 | 64.7 | 527 | 1.692 | 3.4 | 2.744 | −530.78 | 38.5 | 24937.2 |
| 5 mM | | | | | | | | | |
| KNO₃ | — | 79.7 | ND¹ | 2.2697 | 4.5 | 2.744 | −530.78 | ND¹ | ND¹ |
| 2.5 mM | | | | | | | | | |
| NH₄NO₃ | 8 | 75.9 | 762 | 2.1742 | 4.3 | 2.744 | −530.78 | 47.1 | 35782.9 |
| 1.25 mM | | | | | | | | | |
| NH₄NO₃ | 8 | 84.7 | 431 | 2.427 | 4.9 | 2.744 | −530.78 | 35.0 | 29671.3 |
| 2.5 mM | | | | | | | | | |
| NH₄NO₃ | 8 | 87.9 | 245 | 2.6416 | 5.3 | 2.744 | −530.78 | 28.3 | 24867.6 |
| 5 mM | | | | | | | | | |
| NH₄NO₃ | — | 72.4 | ND¹ | 2.1375 | 4.3 | 2.744 | −530.78 | ND¹ | ND¹ |
| 2.5 mM | | | | | | | | | |

*Meja was added once (at D 0)
**Meja was added twice (at D 0 and D 7)
¹“ND” is for Non-detectable
²“FW” is for Fresh Weight

Calculations are as follows (assuming the “Cell culture density” to be 1000 g/L):

$$QS - 18 ( µg \text{/Cell} FW ) = \frac{Sample peak area - Intercept Std curve}{Slope Std curve} \times \frac{1}{Sample inj . vol . ( µL )} \times \frac{Total extr . vol . ( ml )}{Cell FW ( g )} \\ QS - 18 ( µg \text{/L} ) = QS - 18 ( µg \text{/Cell} FW ) \times \frac{P C V ( \% )}{100} \times Cell culture density ( \text{g/L} )$$

TABLE 10
QS-18 volumetric productivity (μg/L) (see last column)/14 days post-elicitation

| | | | Sample | | Total | | | QS-18 | |
|---|---|---|---|---|---|---|---|---|---|
| | | | (20 μl) | Cell | extr. | Slope | Intercept | (μg/g | |
| | | PCV | peak | FW | vol. | Std | Std | cell | QS-18 |
| Nitrogen | MeJa | (%) | area | (g) | (ml) | curve | curve | FW²) | (μg/L) |
| — | 8* | 74.9 | 356.0 | 2.21 | 4.4 | 2.66 | −546.10 | 34.0 | 25451.2 |
| — | 8** | 54.4 | 143.8 | 1.6897 | 3.4 | 2.66 | −546.10 | 26.0 | 14115.0 |
| NH₄Cl | 8 | 77.6 | ND¹ | 2.2401 | 4.5 | 2.66 | −546.10 | ND¹ | ND¹ |
| 1.25 mM | | | | | | | | | |
| NH₄Cl | 8 | 80.6 | 29.5 | 2.1383 | 4.3 | 2.66 | −546.10 | 21.7 | 17463.6 |
| 2.5 mM | | | | | | | | | |
| NH₄Cl | 8 | 54.8 | ND¹ | 1.5155 | 3.0 | 2.66 | −546.10 | ND¹ | ND¹ |
| 5 mM | | | | | | | | | |
| NH₄Cl | — | 77.4 | ND¹ | 1.6947 | 3.4 | 2.66 | −546.10 | ND¹ | ND¹ |
| 2.5 mM | | | | | | | | | |
| KNO₃ | 8 | 79.3 | 377 | 2.1926 | 4.4 | 2.66 | −546.10 | 34.8 | 27574.9 |
| 1.25 mM | | | | | | | | | |
| KNO₃ | 8 | 78.6 | 311 | 2.1642 | 4.3 | 2.66 | −546.10 | 32.3 | 25376.7 |
| 2.5 mM | | | | | | | | | |
| KNO₃ | 8 | 71.0 | 228 | 1.8091 | 3.6 | 2.66 | −546.10 | 29.1 | 20693.8 |
| 5 mM | | | | | | | | | |
| KNO₃ | — | 78.1 | ND¹ | 2.0413 | 4.1 | 2.66 | −546.10 | ND | ND |
| 2.5 mM | | | | | | | | | |
| NH₄NO₃ | 8 | 81.0 | 191 | 2.0176 | 4.0 | 2.66 | −546.10 | 27.8 | 22488.9 |
| 1.25 mM | | | | | | | | | |
| NH₄NO₃ | 8 | 89.9 | 122 | 2.3649 | 4.7 | 2.66 | −546.10 | 25.1 | 22608.6 |
| 2.5 mM | | | | | | | | | |
| NH₄NO₃ | 8 | 89.7 | 176 | 2.5791 | 5.2 | 2.66 | −546.10 | 27.2 | 24398.1 |
| 5 mM | | | | | | | | | |
| NH₄NO₃ | — | 76.2 | ND¹ | 1.745 | 3.5 | 2.66 | −546.10 | ND¹ | ND¹ |
| 2.5 mM | | | | | | | | | |

*Meja was added once (at D 0)
**Meja was added twice (at D 0 and D 7)
¹“ND” is for Non-detectable
²“FW” is for Fresh Weight

Calculations are as follows (assuming the “Cell culture density” to be 1000 g/L):

$$QS - 18 ( µg \text{/Cell} FW ) = \frac{Sample peak area - Intercept Std curve}{Slope Std curve} \times \frac{1}{Sample inj . vol . ( µL )} \times \frac{Total extr . vol . ( ml )}{Cell FW ( g )} \\ QS - 18 ( µg \text{/L} ) = QS - 18 ( µg \text{/Cell} FW ) \times \frac{P C V ( \% )}{100} \times Cell culture density ( \text{g/L} )$$

### Results

Apart from the condition “NH₄Cl 5 mM”, all other reduced concentrations of nitrogen source (i.e. 1.25 mM NH₄Cl, KNO₃ or NH₄NO₃, 2.5 mM NH₄Cl, KNO₃ or NH₄NO₃ and 5 mM KNO₃ or NH₄NO₃) which were tested during the nitrogen depletion phase resulted into saponin production (after elicitation). These results indicate that saponin production may be successfully triggered by elicitation in the presence of a residual concentration of nitrogen source. Complete elimination of nitrogen source from the culture medium is not required prior to elicitation according to the method of the invention.

### 3.10 Experiment 10—QS-21 Volumetric Productivity (Natural Nitrogen Depletion)

The following diagram provides a schematic view illustrative of embodiments of the method of the invention as carried out in this experiment.

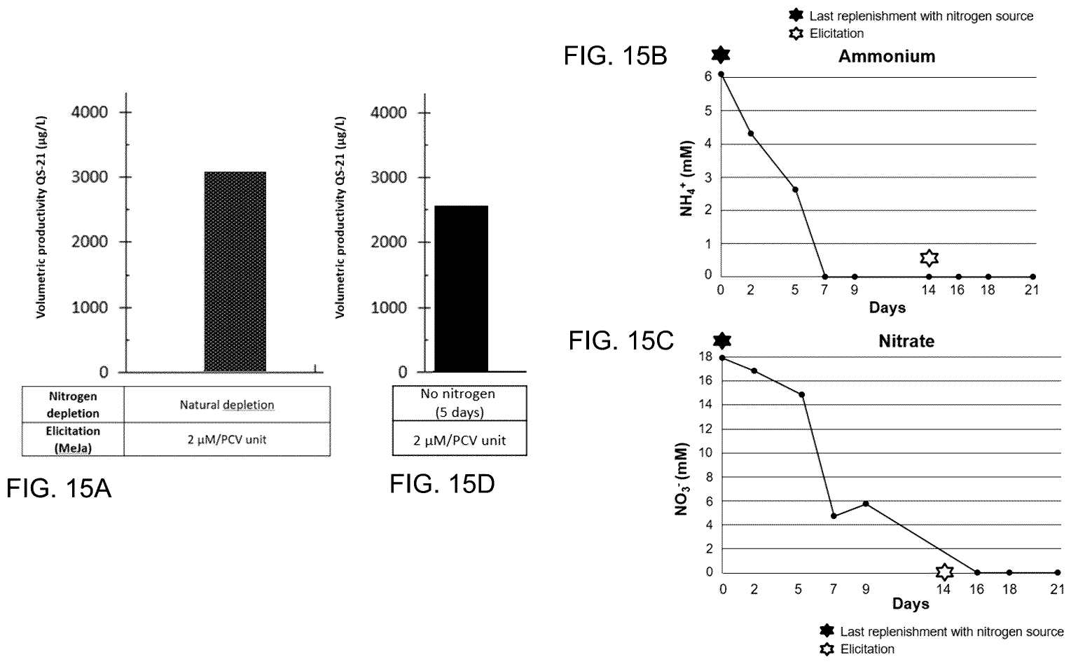

The same suspension cell line (CMC40B6) as used in the above Experiments 1, 2, 4 and 5 was tested, at passage P29. A 10 L bioreactor was seeded with cells at a PCV of about 10% in 8 L of Medium 4 (including 15 mM KNO₃ and 10 mM NH₄NO₃) and cells were grown until reaching a PCV of about 20%. At this point in time, 1 L was collected from the bioreactor to run a side experiment* (see below) and 3 L of Medium 4 (including 15 mM KNO₃ and 10 mM NH₄NO₃) were further added to the remaining 7 L of culture in the bioreactor (i.e. cells were diluted 1.43 fold in the bioreactor, resulting into a PCV of about 14%). This marks the “last replenishment with a nitrogen source” (=DO) in the bioreactor experiment, with no further replenishment of the culture medium with any source of nitrogen. Cells were maintained in the same medium and therefore let naturally consume the nitrogen source included in Medium 4 down to a residual level. The level of ammonium (NH₄⁺ ions) and nitrates (NO₃ ions) was monitored on a regular basis by measuring the respective levels every few days. At D9, the level of ammonium was undetectable and the level of nitrates was about 5 mM (see FIG. 15B, and 15C, respectively). Cells were further maintained in the same medium for 5 more days. 2 μM/PCV % MeJa was then added (D14). 7 days post-elicitation (i.e. at D21), the cells were harvested and disrupted (as described in Experiment 5), and the saponin content in the plant cell extracts was measured (as described in Example 6). QS-21 volumetric productivity was looked at. The data are presented in Table 11 below and the results in the form of a graph in FIG. 15A.

### Glucose and Phosphate (PO₄) Feed Conditions During the Nitrogen Depletion and Elicitation Phases:

The minimum target for glucose level and phosphate (PO₄) level during both the nitrogen depletion phase and the elicitation phase was 15 mM and 0.6 mM, respectively. Glucose level and phosphate (PO₄) level were measured every few days. In case the measured level of glucose was lower than 15 mM, the culture medium was fed with a 60 mM glucose solution. In case the measured level of phosphate (PO₄) was lower than 0.6 mM, the culture medium was fed with a 2.5 mM phosphate solution.

### *Side Experiment

The 1 L collected from the bioreactor as referred to above was centrifuged and resuspended into Medium 1 (no source of nitrogen) at a PCV between 20-25% (this is Day 0). 5 days later, at Day 5, the PCV % of each culture was measured and 2 μM/PCV % MeJa was added directly to the culture medium (i.e. after Meja was added) for 7 days. Cells were then harvested and disrupted (as described in Experiment 6), and the saponin content in the plant cell extract was measured (as described in Example 6). QS-21 volumetric productivity was looked at. The results are presented in the form of a graph in panel D of FIG. 15 (data are not shown).

TABLE 11
QS-21 volumetric productivity (μg/L) (see last column)

| | | | QS-21 | | | | | |
|---|---|---|---|---|---|---|---|---|
| | | Sample | Std | QS-21 | | Total | QS-21 | |
| | | (1 μl) | (1 μL) | Std | Cell | extr. | (μg/g | |
| | PCV | peak | peak | Conc. | FW¹ | vol. | Cell | QS-21 |
| Cell line | (%) | area | area | (μg/ml) | (g) | (mL) | FW¹) | (μg/L) |
| CMC40B6 | 75 | 3477.33 | 4369 | 10 | 1.9315 | 1 | 4.13 | 3095.1123 |

¹“FW” is for Fresh Weight

Calculations are as follows (assuming the “Cell culture density” to be 1000 g/L):

$$QS - 21 ( µg \text{/Cell} FW ) = Sample peak area \times \frac{QS - 21 Std conc . ( µg / ml )}{QS - 21 Std peak area} \times \frac{QS - 21 Std inj . vol . ( µL )}{Sample inj . vol . ( µL )} \times \frac{Total extr . vol . ( ml )}{Cell FW ( g )} \\ QS - 21 ( µg \text{/L} ) = QS - 21 ( µg \text{/Cell} FW ) \times \frac{P C V ( \% )}{100} \times Cell culture density ( \text{g/L} )$$

### Results

FIG. 15A indicates that natural nitrogen depletion (i.e. letting the cells naturally consume the nitrogen source present in the culture medium), prior to elicitation, also leads to saponin production (as exemplified here by looking at QS-21 saponins).

FIG. 15D indicates that QS-21 volumetric productivity achieved by natural depletion is within the same range as the volumetric productivity achieved when nitrogen depletion is performed by removing the culture medium and replacing it with a culture medium containing no nitrogen source.

### 3.11 Conclusion

Saponin production (e.g. QS-7, QS-21 and QS-18 saponins) was reproducibly observed and obtained using at least 4 different suspension cell lines used at different passages, when using methods in accordance with the invention. Different nitrogen depletion conditions and different elicitation conditions with varying concentrations of elicitor similarly resulted in saponin production, regardless of the extraction process used and as confirmed by different analytical methods.

### Example 4—Extraction of Saponins

At the end of the elicitation, the PCV of the cultures to be extracted was measured. About 3 ml of each suspension culture were transferred to 7 ml-tubes pre-filled with Precellys® ceramic beads. Tubes were centrifuged for 5 min at 1000 g. After centrifugation, the supernatant was discarded. After weighing the cell pellet (“cell fresh weight” or “cell FW”), a volume of sodium acetate buffer (30 mM, pH 6) equivalent to the volume of the cell pellet was added to the cell pellet. Based on the assumption that 1 g of cell pellet equates to 1 mL of cell pellet, the “total extraction volume” in Tables 2, 3, 7, 8, 9 and 10 represents the sum of the sodium acetate buffer volume added and the cell FW. Cell pellets were then homogenized using Precellys® equipped with Cryolis using the following conditions maintained at 4° C.: 3 cycles of 30 sec at 8000 rpm with a pause interval of 60 sec between each cycle. Tubes were then centrifuged 10 min at 5000 g at 4° C. Supernatants were then filtered on a train filter made of Millex-HV PVDF 0.45 μm and Millex-HV PVDF 022 μm filters. After filtration, the extracts are ready for dosing the saponin content, and may be stored at 4° C. before analysis. The saponin content was dosed in a 20 μl sample of the plant cell extracts to be analyzed according to the method described in Example 5.

### Example 5—Analysis of Saponin Content by HPLC/ELSD

The saponin content in plant cell extracts was measured by HPLC/ELSD, using the following parameters:
Chromatographic System: Agilent 1290LC
Column: Waters Acquity BEH C18, 2.1 mm×100 mm; 1.7 μm 130A
Oven temp: 55° C.
Autosampler temp: 10° C.
Flow rate: 0.6 ml/min
Run Time: 11.00 min
Injection Volume: 20 μl
Mobile Phase A: H₂O/ACN/IPA (75/20/5) v/v/v 0.025% FA
Mobile Phase B: H₂O/ACN/IPA (10/72/18) v/v/v 0.025% FA

### Gradient:

| Time | | |
|---|---|---|
| (min) | % Mobile Phase A | % Mobile Phase B |
| 0 | 100 | 0 |
| 6.23 | 23 | 77 |
| 6.4 | 0 | 100 |
| 7.1 | 0 | 100 |
| 7.3 | 100 | 0 |
| 7.31 | 100 | 0 |
| 8.3 | 0 | 100 |
| 9.29 | 0 | 100 |
| 9.3 | 100 | 0 |
| 10 | 100 | 0 |
| 11 | 100 | 0 |

| | |
|---|---|
| Time (min) | Column compartment event |

| | | |
|---|---|---|
| Change valve position | Position (waste) | 1 |
| Change valve position | Position (Elsd Detector) | 2 |
| Change valve position | Position (Waste) | 1 |

ELSD Detection parameters:
Evaporator: 80° C.
Nebulisator: 90° C.
Nitrogen flow: 1.5 SLM

### Retention Time

### QS-17

A standard (50 μg/ml) corresponding to the QS-17 fraction isolated and purified from a crude bark extract of Quillaja Saponaria trees has been used (“QS-17 standard”) to establish a calibration curve, allowing the subsequent quantification of QS-17 saponin family present in the plant cell extract. The retention time at which the QS-17 standard peaked by HPLC/ELSD is about 4.32 min (data not shown).

### QS-21

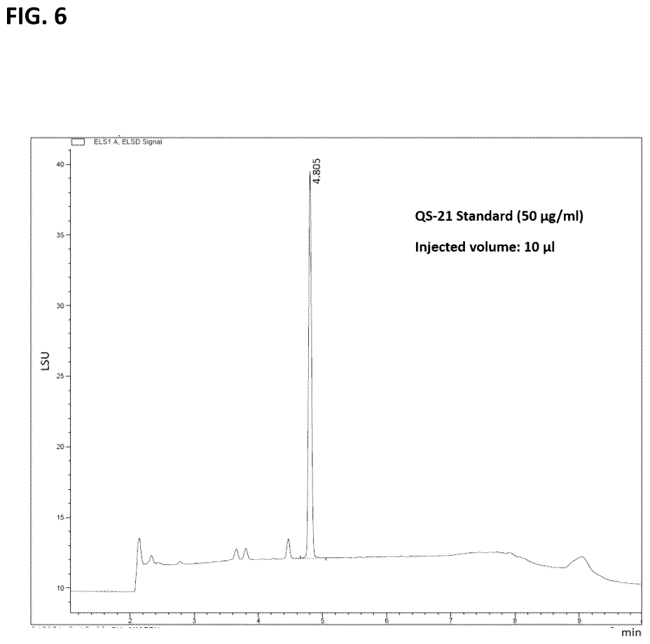

A standard (50 μg/ml) corresponding to the QS-21 fraction isolated and purified from a crude bark extract of Quillaja Saponaria trees has been used (“QS-21 standard”) to establish a calibration curve, allowing the subsequent quantification of QS-21 saponin family present in the plant cell extract. The QS-21 standard was obtained using the purification method described in Example 3 of WO 19/10692. The retention time at which the QS-21 standard peaked by HPLC/ELD is about 4.80 min (see FIG. 6).

### QS-18

A standard (50 μg/ml) corresponding to the QS-18 fraction isolated and purified from a crude bark extract of Quillaja Saponaria trees has been used (“QS-18 standard”) to establish a calibration curve, allowing the subsequent quantification of QS-18 saponin family present in the plant cell extract.

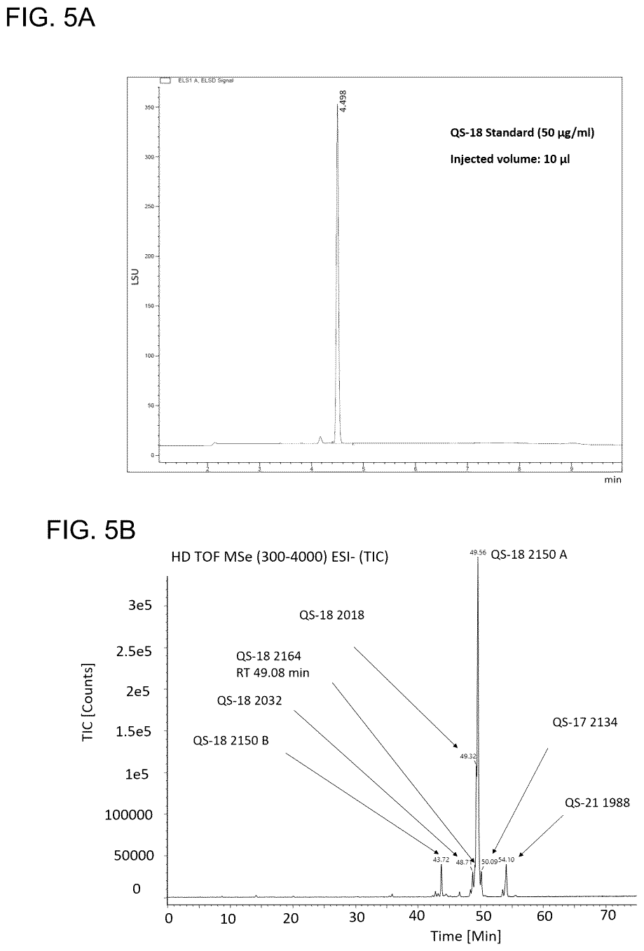

The QS-18 standard was obtained using the purification method described in the Example 3 of WO 19/10692 and as follows: QS-18-containing phenyl fractions, following the reverse phase chromatography using a phenyl resin (EPDM), were collected (the presence of m/z corresponding to key components was confirmed by MS—data not shown). The retention time at which the QS-18 standard peaked by HPLC/ELSD is about 4.54 min (see FIG. 5A).

The amount of QS-18 saponins in a given plant cell extract was determined by comparing the peak area obtained for the plant cell extract with the peak area obtained for the QS-18 standard. Taking into account the PCV % measured before harvesting and extraction, the cell fresh weight in the plant cell extract and the total extraction volume, the amount of QS-18 saponins in a given plant cell extract was converted into QS-18 volumetric productivity expressed in μg/L (on the assumption that 1 g of fresh cell weight equates to 1 ml). Details of calculation are provided in the above Tables reporting the data used for the calculation.

### Composition of the QS-18 Standard

The identity of the saponin species included in the QS-18 standard has been analyzed in parallel using a 120 min high-resolution LCMS mass spec method using Qtof mass spectrometer, looking at all saponin species having a monoisotopic molecular weight (m/z) ranging from 300 to 4000. As provided in the chromatogram shown in FIG. 5B, the major saponin species in the QS-18 standard is QS-18 2150 A (V1 & V2). It also includes minor saponin species, such as QS-18 2150 B (V1 & V2), QS-18 2032, QS-18 2164, QS-18 2018, QS-17 2134, and QS-21 1988.

### Example 6—Analysis of Saponin Content by LC-MS/MS

The saponin content in plant cell extracts was alternatively measured by LCMS/MS, using the following parameters:

| Parameter | Conditions |
|---|---|
| Instrument | Waters Acquity H class coupled to a Waters |
| | Xevo Tandem Quadrupole (TQD) Mass |
| | Spectrometer |

UPLC Conditions

| Column | Waters BEH C18 1.7 um 2.1 × 50 mm | | |
|---|---|---|---|
| Temperature | 40° C. ± 3° C. | | |
| Flow Rate | 0.5 mL/min | | |
| Gradient Curve | 6 | | |
| Injection Volume | 1 to 10 μL dependent on concentration | | |
| Autosampler Temperature | 10° C. ± 3° C. | | |
| Run Time | 3 mins | | |
| Post-Inject Wash | 50% methanol | | |
| Mobile Phase A | 0.1% Formic Acid in Water | | |
| Mobile Phase B | 0.1% Formic Acid in Acetonitrile | | |
| Syringe Wash Solvent | 50% Methanol | | |
| Seal Wash | 50% Methanol | | |
| Purge Solvent | 50% Acetonitrile | | |
| Gradient | Time | % A | % B |
| | 0 | 55 | 45 |
| | 0.75 | 55 | 45 |
| | 2.00 | 5 | 95 |
| | 2.1 | 5 | 95 |
| | 2.2 | 55 | 45 |
| | 3.0 | 55 | 45 |

Mass Spec Conditions - or similar to give maximum sensitivity

| | | | | | Extended | | |
|---|---|---|---|---|---|---|---|
| Source | | | Analyser | | Tab | | |
| Capillary | 3.9 | kV | LM | 11.6 | Source | 150° | C. |
| Voltage | | | Resolution 1 | | Temp | | |
| Cone | 36 | V | HM | 14.9 | Extractor | 3 | V |
| | | | Resolution1 | | | | |
| Desolvation | 500° | C. | Ion Energy 1 | 0.4 | RF Lens | 2.5 | V |
| Temperature | | | | | | | |
| Source | 150° | C. | LM | 12.2 | Entrance | 30 | |
| Temperature | | | Resolution 2 | | | | |
| Source Gas | | | HM | 14.8 | Exit | 30 | |
| Flow | | | Resolution 2 | | | | |
| Desolvation | 1000 | L/Hr | Ion Energy 2 | 0.8 | Gain | 1 | |
| Gas Flow | | | | | | | |

| | | | |
|---|---|---|---|
| Cone (L/Hr) | 0 | Collision | 3 |

Energy

| | |
|---|---|
| Collision Gas | Argon |

| | | Dwell | | Cone | Collision |
|---|---|---|---|---|---|
| | | Time | | Voltage | Energy |
| Component | Mode | (secs) | MRM Transitions | (V) | (v) |
| QS-21 1988 | ESI - | Auto | 993.46 > 755.5 | 34 | 16 |
| QS-18 2150 | ESI - | Auto | 1074.50 > 836.4 | 34 | 16 |

A QS-21 fraction isolated and purified from a crude bark extract of Quillaja Saponaria trees using the purification method described in Example 3 of WO 19/10692 has been used as a standard (“QS-21 standard”—same as in Example 5).

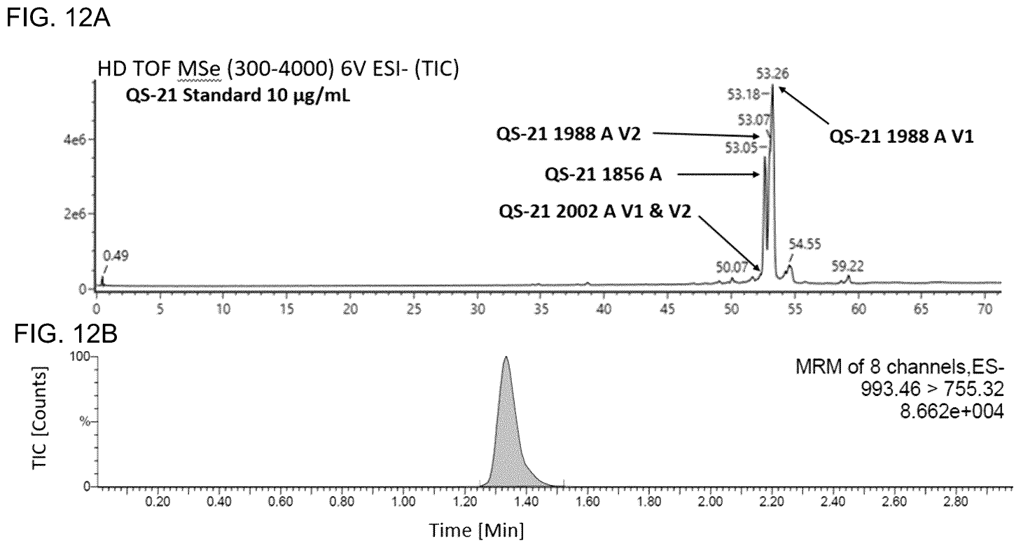

The amount of QS-21 saponins in a given plant cell extract was determined by comparing the peak area obtained for the plant cell extract with the peak area obtained for the QS-21 standard (10 μg/ml), at the above MRM 993.46>755.55 transition (corresponding to QS-21 1988). A representative chromatogram is provided in FIG. 12B. Taking into account the PCV % measured before harvesting and extraction, the cell fresh weight in the plant cell extract (or the volume of the sample to be analysed—“Vol. PCC”) and the total extraction volume, the amount of QS-21 saponins in a given plant cell extract was converted into QS-21 volumetric productivity expressed in μg/L (on the assumption that 1 g of fresh cell weight equates to 1 ml). Details of calculation are provided in the above Tables reporting the data used for the calculation.

### Composition of the QS-21 Standard

The identity of the saponin species included in the QS-21 standard has been analyzed in parallel using a 120 min high-resolution LCMS mass spec method using Qtof mass spectrometer, looking at all saponin species having a monoisotopic molecular weight (m/z) ranging from 300 to 4000. As provided in the chromatogram shown in FIG. 12A, the major saponin species in the QS-21 standard are QS-21 1988 A V1 and QS-21 1988 A V2. It also includes minor saponin species, such as QS-21 2002 A V1 and QS-21 2002 A V1.

### REFERENCES

Reichert et al. in 2019 (“Quillaja Saponin Characteristics and Functional Properties”; Annu Rev Food Sci Technol. March 25; 10, p 43-73)
Dalsgaard et al. in 1974 (“Saponin adjuvants”; Archiv. Fur die gesamte Virusforschung, Vol. 44, Springer Verlag, Berlin, p 243-254)
Kensil et al. in 1991 (“Separation and characterization of saponins with adjuvant activity from Quillaja 87aponaria Molina cortex”; Journal of immunology, Vol. 146: p 431-437)
Ragupathi, G. et al. 2011 (“Natural and synthetic saponin adjuvant QS-21 for vaccines against cancer”; Expert Review of Vaccines, Vol. 10: p 463-470)
Garcon, N. et al. in 2011 (“Recent clinical experience with vaccines using MPL and QS-21-containing adjuvant systems”; Expert Review of Vaccines, Vol. 10(4): p 71-486)
Didierlaurent, A. et al. in 2017 (“Adjuvant system ASO1: helping to overcome the challenges of modern vaccines”; Expert Review of Vaccines, Vol. 16(1): p 55-63)
Didierlaurent A., et al. in 2014 (“Enhancement of Adaptive Immunity by the Human Vaccine Adjuvant ASO1 Depends on Activated Dendritic Cells”; Journal of Immunology, Vol. 193(4): p 1920-1930).
De Becker, G. et al. in 2000 (“The adjuvant monophosphoryl lipid A increases the function of antigen-presenting cells”; International immunology, Vol. 12: p 807-815)
Ismaili, et al. in 2002 (“Monophosphoryl lipid A activates both human dendritic cells and T cells”; Journal of immunology, Vol. 168(9): p 26-932)
Martin, M. et al. in 2003 (“Role of innate immune factors in the adjuvant activity of monophosphoryl lipid A”; Infection and immunity, Vol (71): p 2498-2507)
Mata-Haro, V. et al. in 2007 (“The vaccine adjuvant monophosphoryl lipid A as a TRIF-biased agonist of TLR4”; Science, Vol. (316): p 1628-1632)
Kensil, C. et al. in 1998 (“QS-21: a water-soluble triterpene glycoside adjuvant”; Expert Opinion on Investigational Drugs, Vol. 7: p 1475-1482)
Newman, M. J. et al. in 1992 (“Saponin adjuvant induction of ovalbumin-specific CDS+ cytotoxic T lymphocyte responses”; Journal of immunology, Vol. 148: p 2357-2362)
Soltysik, S. et al. in 1995 (“Structure/function studies of QS-21 adjuvant: assessment of triterpene aldehyde and glucuronic acid roles in adjuvant function”; Vaccine, Vol. 13: p 1403-1410)
Lambrecht, B. N. et al. in 2009 (“Mechanism of action of clinically approved adjuvants”; Current Opinion in Immunology, Vol. 21: p 23-29)
Li, H., S. B. et al. in 2008 (“Cutting edge: inflammasome activation by alum and alum's adjuvant effect are mediated by NLRP3”; Journal of Immunology, Vol. 181: p 17-21)
Marty-Roix, R. et al. in 2016 (“Identification of QS-21 as an Inflammasome-activating Molecular Component of Saponin Adjuvants”; J. Biol. Chem. Vol. 291: p 1123-36)
Yendo, A et al. in 2010 (“Production of Plant Bioactive Triterpenoid Saponins: Elicitation Strategies and Target Genes to Improve Yields”; Mol. Biotech. Vol. 46: p 94-104)
US 2019/0134128
WO 2011/161151
WO 2015/082978
WO 2019/106192
WO 2013/041572

1. A method for producing saponins, the saponins comprising a quillaic acid triterpenoid aglycone, said method comprising at least the following steps:
i) culturing plant cells in a culture medium comprising a nitrogen source, the plant cells being capable of naturally synthesizing the saponin;
ii) depleting the culture medium from any nitrogen source;
iii) eliciting the production of the saponins with at least one elicitor; and
iv) recovering the saponins produced.

2. The method according to claim 1, wherein the plant cells are suspension cell lines.

3. The method according to claim 1, wherein the total concentration of the nitrogen source in the culture medium in step i) is from 10 mM to 50 mM.

4. The method according to claim 1, wherein the culture medium in step i) comprises one or more of sucrose, glucose, and fructose as carbon source.

5. The method according to claim 1, wherein step ii) is performed by replacing the culture medium at the end of step i) with a culture medium, containing no source of nitrogen and maintaining the plant cells in the replacing culture medium.

6. The method according to claim 3, wherein step ii) is performed by replacing the culture medium at the end of step i) with a culture medium containing from 1.25 mM to 5 mM of the nitrogen source and maintaining the plant cells in the replacing culture medium.

7. The method according to claim 1, wherein step ii) is from 1 to 9 days.

8. The method according to claim 3, wherein step ii) is performed by letting the plant cells naturally consume the nitrogen source included in the culture medium in step i) down to a residual level, with no further replenishment of the culture medium with any nitrogen source, and maintaining the plant cells in the consumed culture medium.

9. The method according to claim 8, wherein the residual level of the nitrogen source is less than 10 mM.

10. The method according to claim 8, wherein step ii) is from 5 to 20 days.

11. The method according to claim 8, wherein the plant cells are maintained in the consumed culture medium from 1 to 9 days.

12. The method according to claim 1, wherein the at least one elicitor in step iii) is a moncocarboxylic compound-type elicitor.

13. The method according to claim 12, wherein the at least one elicitor is methyl jasmonate.

14. The method according to claim 1, wherein the at least one elicitor in step iii) is added directly to the plant cells at the end of step ii).

15. The method according to claim 1, wherein step iii) is from 1 to 14 days.

16. A suspension cell line of plant cells naturally synthesizing quillaic acid-based triterpenoid saponins capable of producing the quillaic acid-based triterpenoid saponins at a volumetric productivity of at least 5 mg/L.

17. The method according to claim 1, wherein the plant cells are from the genus Quillaja.

18. The method according to claim 17, wherein the plant cells are from Quillaja saponaria.

19. The method according to claim 17, wherein the saponins are one or more saponin species from the QS-7 saponin family, the QS-17 saponin family, the QS-18 saponin family, the QS-21 saponin family, and combinations thereof.

20. The method according to claim 19, wherein the saponins are one or more saponin species from the QS-21 saponin family.

21. The method according to claim 20, wherein the saponins are one or more of: QS-21 1988 A V1, QS-21 1988 A V2, QS-21 1988 B V1, and QS-21 1988 B V2.

22. A method for preparing an adjuvant comprising saponins, said method comprising the following steps:
a) preparing saponins according to the method of claim 1, and
b) formulating the recovered saponin as an adjuvant.

23. The suspension cell line according to claim 16, wherein the plant cells are from the genus Quillaja.

24. The suspension cell line according to claim 23, wherein the plant cells are from Quillaja saponaria.

25. The suspension cell line according to claim 23, wherein the saponins are one or more saponin species from the QS-7 saponin family, the QS-17 saponin family, the QS-18 saponin family, the QS-21 saponin family, and combinations thereof.

26. The suspension cell line according to claim 25, wherein the saponins are one or more saponin species from the QS-21 saponin family.

27. The suspension cell line according to claim 26, wherein the saponins are one or more of: QS-21 1988 A V1, QS-21 1988 A V2, QS-21 1988 B V1, and QS-21 1988 B V2.
